(19) United States
(12) Patent Application Publication
(10) Pub. No.: US 2024/0150837 A1
(43) Pub. Date: May 9, 2024

(54) METHODS AND SYSTEMS FOR METHYLATION PROFILING OF PREGNANCY-RELATED STATES

(71) Applicant: Mirvie, Inc., South San Francisco, CA (US)

(72) Inventors: Eugeni Namsaraev, Santa Clara, CA (US); Arkady Khodursky, South San Francisco, CA (US); Mitsu Reddy, South San Francisco, CA (US)

(21) Appl. No.: 18/508,732

(22) Filed: Nov. 14, 2023

Related U.S. Application Data
(63) Continuation of application No. PCT/US2022/029560, filed on May 17, 2022.
(60) Provisional application No. 63/189,958, filed on May 18, 2021.

(51) Int. Cl. C12Q 1/6883; G16B 20/00; G16B 40/20; G16H 10/60; G16H 50/20; G16H 50/30

(52) U.S. Cl. CPC C12Q 1/6883; G16B 20/00; G16B 40/20; G16H 10/60; G16H 50/20; G16H 50/30; C12Q 2600/118; C12Q 2600/154; C12Q 2600/158

(57) ABSTRACT

The present disclosure provides methods and systems directed to methylation profiling for cell-free identification and/or monitoring of pregnancy-related states. A method for identifying or monitoring a presence or susceptibility of a pregnancy-related state of a subject may comprise assaying a cell-free biological sample derived from said subject to detect a set of biomarkers, and analyzing the set of biomarkers with a trained algorithm to determine the presence or susceptibility of the pregnancy-related state.

### CROSS-REFERENCE

This application is a continuation of International Application No. PCT/US2022/029560, filed May 17, 2022, which claims the benefit of U.S. Application No. 63/189,958, filed May 18, 2021, each of which is incorporated by reference herein in its entirety.

### BACKGROUND

Every year, about 15 million pre-term births are reported globally, and over 300,000 women die of pregnancy related complications such as hemorrhage and hypertensive disorders like preeclampsia. Pre-term birth may affect as many as about 10% of pregnancies, of which the majority are spontaneous pre-term births. Pregnancy-related complications such as pre-term birth are a leading cause of neonatal death and of complications later in life. Further, such pregnancy-related complications may cause negative health effects on maternal health.

### SUMMARY

Currently, there may be a lack of meaningful, clinically actionable diagnostic screenings or tests available for many pregnancy-related complications such as pre-term birth. Thus, to make pregnancy as safe as possible, there exists a need for rapid, accurate methods for identifying and monitoring pregnancy-related states that are non-invasive and cost-effective, toward improving maternal and fetal health.

The present disclosure provides methods, systems, and kits for identifying or monitoring pregnancy-related states by processing cell-free biological samples obtained from or derived from subjects. Cell-free biological samples (e.g., plasma samples) obtained from subjects may be analyzed to identify the pregnancy-related state (which may include, e.g., measuring a presence, absence, or relative assessment of the pregnancy-related state). Such subjects may include subjects with one or more pregnancy-related states and subjects without pregnancy-related states. Pregnancy-related states may include, for example, pre-term birth, full-term birth, gestational age, due date (e.g., due date for an unborn baby or fetus of a subject), onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), placenta accreta spectrum disorders (placenta increta, placenta percreta, and placenta accreta), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and fetal development stages or states (e.g., normal fetal organ function or development, and abnormal fetal organ function or development). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

In an aspect, the present disclosure provides a method for identifying a presence or susceptibility of a pregnancy-related state of a subject, comprising assaying DNA methylation states and/or RNA transcripts in a cell-free biological sample derived from the subject to detect a set of biomarkers, and analyzing the set of biomarkers with a trained algorithm to determine the presence or susceptibility of the pregnancy-related state.

In some embodiments, the method further comprises assaying the DNA methylation states in the cell-free biological sample derived from the subject to detect the set of biomarkers. In some embodiments, the method further comprises assaying 5-methylcytosine (5mC) and/or 5-hydroxymethylcytosine (5hmC) in the cell-free biological sample derived from the subject to detect the set of biomarkers. In some embodiments, the method further comprises assaying the DNA methylation state by bisulfite sequencing. In some embodiments, the method further comprises assaying the hydroxymethylated cell-free DNA in the cell-free biological sample derived from the subject to detect the set of biomarkers. In some embodiments, the changes of DNA methylation states are assayed by enriching 5hmC containing DNA with subsequent nucleic acid sequencing. In some embodiments, the 5hmC containing DNA fragments are enriched by affinity based methods. In some embodiments, 5hmC containing DNA fragments are enriched by anti-5hmC antibodies or, after treatment with sodium bisulfite, by anti-CMS antibody. In some embodiments, 5hmC containing DNA fragments are labeled by glucose by beta.-glucosyltransferase (OGT). In some embodiments, the resulting glucosylated 5hmC is enriched with JBP-1. In some embodiments, the βGT-treated 5hmC undergoes glucosylation, periodate oxidation, or biotinylation (GLIB). For example, in this reaction, sodium periodate may cleave the vicinal hydroxyl groups in the glucose to generate reactive aldehyde groups, which may be biotinylated using an aldehyde-reactive hydroxylamine-biotin probe. In some embodiments, biotinylated 5hmC residues is enriched using streptavidin beads. In some embodiments, an azide-modified glucose is introduced to 5hmC by βGT and subsequently biotinylated via click chemistry in selective chemical labeling (hMe-Seal). In some embodiments, biotinylated 5hmC residues are enriched using streptavidin beads. In some embodiments, βGT and UDP are glucose modified with a chemoselective group, thereby covalently labeling the hyroxymethylated DNA molecules in the cfDNA with the chemoselective group, and a biotin moiety is linked to the chemoselectively-modified cfDNA via a cycloaddition reaction. In some embodiments, the DNA methylation states or the changes in methylation states are assayed with nucleic acid sequencing. In some embodiments, the changes of DNA methylation states are assayed by subsequent nucleic acid detection by hybridization to array or qPCR.

In another aspect, the present disclosure provides a method for identifying the presence of changes in a DNA methylation state associated with the changes in RNA transcript expression level encoded by the same gene region. In some embodiments, the changes of methylation DNA in corresponding gene regions are assayed by 5hmC DNA enrichment. In some embodiments, the DNA methylation gene region comprises promoter sequences, exonic sequences, and/or intronic sequences.

In another aspect, the present disclosure provides a method for identifying the presence of changes in the DNA methylation state associated with pregnancy-related state in genomic regions outside of the gene coding regions. In some embodiments, the methylation DNA region includes 5′ and 3′ untranslated gene regions, transcription factor binding sites, CpG islands, promoter and enhancer regions.

In another aspect, the present disclosure provides a method for identifying a presence or susceptibility of a pregnancy-related state of a subject, comprising assaying a cell-free biological sample derived from the subject to detect a set of biomarkers, and analyzing the set of biomarkers with a trained algorithm to determine the presence or susceptibility of the pregnancy-related state among a set of at least three distinct pregnancy-related states at an accuracy of at least about 80%.

In some embodiments, the pregnancy-related state is selected from the group consisting of pre-term birth, full-term birth, gestational age, due date, onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), placenta accreta spectrum disorders (placenta increta, placenta percreta, and placenta accreta), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and fetal development stages or states (e.g., normal fetal organ function or development, and abnormal fetal organ function or development). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

In some embodiments, the pregnancy-related state is a sub-type of pre-term birth, and the at least three distinct pregnancy-related states include at least two distinct sub-types of pre-term birth. In some embodiments, the sub-type of pre-term birth is a molecular sub-type of pre-term birth, and the at least two distinct sub-types of pre-term birth include at least two distinct molecular sub-types of pre-term birth. In some embodiments, the distinct molecular subtypes of pre-term birth comprise a molecular subtype of pre-term birth selected from the group consisting of presence or history of prior pre-term birth, presence or history of spontaneous pre-term birth, presence or history of late miscarriage, presence or history of receiving cervical surgery, presence or history of a uterine anomaly, presence or history of ethnicity specific pre-term birth risk (e.g., among an African-American population), and presence or history of pre-term premature rupture of membrane (PPROM).

In some embodiments, the pregnancy-related state is a sub-type of preeclampsia, and the at least three distinct pregnancy-related states include at least two distinct sub-types of preeclampsia. In some embodiments, the distinct molecular subtypes of preeclampsia comprise a molecular subtype of preeclampsia selected from the group consisting of: presence or history of chronic or pre-existing hypertension, presence or history of gestational hypertension, presence or history of mild preeclampsia (e.g., with delivery greater than 34 weeks gestational age), presence or history of severe preeclampsia (with delivery less than 34 weeks gestational age), presence or history of eclampsia, and presence or history of HELLP syndrome.

In some embodiments, the method further comprises identifying pregnancy-related states that are indicative of an impact to postpartum health (e.g., short-term or long-term) of a maternal, newborn, infant, or offspring subject. In some embodiments, pregnancy-related states of a maternal subject comprise postpartum health conditions that develop later (e.g., 5 to 15 years postpartum) such as hypertension, diabetes, and cardiovascular diseases. In some embodiments, health-related conditions of a newborn, infant, or offspring subject comprise health conditions that develop later (e.g., 5 to 15 years postpartum) such as hypertension, diabetes, cardiovascular diseases, and neurological development.

In some embodiments, the method further comprises identifying a clinical intervention for the subject based at least in part on the presence or susceptibility of the pregnancy-related state. In some embodiments, the clinical intervention is selected from a plurality of clinical interventions. In some embodiments, the method further comprises determining a likelihood of the determination of the susceptibility of the pregnancy-related state of the subject, after which subject may be provided with the clinical intervention. In some embodiments, the clinical intervention comprises a pharmacological, surgical, or procedural treatment to reduce severity, delay, or eliminate the future susceptibility pregnancy-related state of the subject (e.g., aspirin for preeclampsia and steroids for pre-term birth).

In some embodiments, the set of biomarkers comprises a locus (e.g., genomic locus) associated with gestational age, wherein the locus is selected from the group consisting of genes listed in Table 3, non-genic loci listed in Table 4, genes listed in Table 5, non-genic loci listed in Table 6, genes listed in Table 7, and genes listed in Table 9. In some embodiments, the panel of the one or more loci comprises a locus associated with preeclampsia, wherein the locus is selected from the group consisting of genomic and non-genomic or aggregated loci listed in Table 11, Table 12, Table 13, and the CLDN7, PAPPA2, SNORD14A, PLEKHH1, MAGEA10, TLE6, and FABP11 genes.

In some embodiments, the set of biomarkers comprises at least 5 distinct loci. In some embodiments, the set of biomarkers comprises at least 10 distinct loci. In some embodiments, the set of biomarkers comprises at least 25 distinct loci. In some embodiments, the set of biomarkers comprises at least 50 distinct loci. In some embodiments, the set of biomarkers comprises at least 100 distinct loci. In some embodiments, the set of biomarkers comprises at least 150 distinct loci.

In some embodiments, the set of biomarkers comprises differentially methylated sites (e.g., hypomethylated and/or hypermethylated). In some embodiments, the set of biomarkers comprises at least 5 distinct differentially methylated sites. In some embodiments, the set of biomarkers comprises at least 10 distinct differentially methylated sites. In some embodiments, the set of biomarkers comprises at least 25 distinct differentially methylated sites. In some embodiments, the set of biomarkers comprises at least 50 distinct differentially methylated sites. In some embodiments, the set of biomarkers comprises at least 100 distinct differentially methylated sites. In some embodiments, the set of biomarkers comprises at least 150 distinct differentially methylated sites.

In some embodiments, the set of biomarkers comprises proteins or protein constituents corresponding to a set of loci. In some embodiments, the set of biomarkers comprises at least 10 distinct proteins or protein constituents. In some embodiments, the set of biomarkers comprises at least 25 distinct proteins or protein constituents. In some embodiments, the set of biomarkers comprises at least 50 distinct proteins or protein constituents. In some embodiments, the set of biomarkers comprises at least 100 distinct proteins or protein constituents. In some embodiments, the set of biomarkers comprises at least 150 distinct proteins or protein constituents.

In another aspect, the present disclosure provides a method comprising assaying a cell-free biological sample derived from a subject; identifying the subject as having or at risk of having preeclampsia; and upon identifying the subject as having or at risk of having preeclampsia, administering an anti-hypertensive drug to the subject.

In another aspect, the present disclosure provides a method for identifying or monitoring a presence or susceptibility of a pregnancy-related state of a subject, comprising: (a) using a first assay to process a cell-free biological sample derived from the subject to generate a first dataset comprising RNA transcriptional biomarkers; (b) using a second assay to process a cell-free biological sample derived from the subject to generate a second dataset comprising DNA methylation state biomarkers; (c) computer processing (e.g., a trained algorithm) at least the first dataset and the second dataset to determine the presence or susceptibility of the pregnancy-related state, which trained algorithm has an accuracy of at least about 80%; and (d) electronically outputting a report indicative of the presence or susceptibility of the pregnancy-related state of the subject.

In some embodiments, the first assay comprises using cell-free ribonucleic acid (cfRNA) molecules derived from the cell-free biological sample to generate the first dataset. In some embodiments, the second assay comprises using cell-free deoxyribonucleic acid (cfDNA) molecules derived from the cell-free biological sample to generate the second dataset.

In some embodiments, the first dataset comprises a first set of biomarkers associated with the pregnancy-related state. In some embodiments, the second dataset comprises a second set of biomarkers associated with the pregnancy-related state. In some embodiments, the second set of biomarkers is different from the first set of biomarkers.

In some embodiments, the pregnancy-related state is selected from the group consisting of pre-term birth, full-term birth, gestational age, due date, onset of labor, pregnancy-related hypertensive disorders, preeclampsia, eclampsia, gestational diabetes, a congenital disorder of a fetus of the subject, ectopic pregnancy, spontaneous abortion, stillbirth, post-partum complications, hyperemesis gravidarum (morning sickness), hemorrhage or excessive bleeding during delivery, premature rupture of membrane, premature rupture of membrane in pre-term birth, placenta previa (placenta covering the cervix), placenta accreta spectrum disorders (placenta increta, placenta percreta, and placenta accreta), intrauterine/fetal growth restriction, macrosomia (large fetus for gestational age), neonatal conditions, and fetal development stages or states.

In some embodiments, the pregnancy-related state comprises pre-term birth. In some embodiments, the pregnancy-related state comprises gestational age. In some embodiments, the pregnancy-related state comprises preeclampsia.

In some embodiments, the cell-free biological sample is selected from the group consisting of cell-free ribonucleic acid (cfRNA), cell-free deoxyribonucleic acid (cfDNA), cell-free fetal DNA (cffDNA), plasma, serum, urine, saliva, amniotic fluid, and derivatives thereof. In some embodiments, the cell-free biological sample is obtained or derived from the subject using an ethylenediaminetetraacetic acid (EDTA) collection tube, a cell-free RNA collection tube, or a cell-free DNA collection tube. In some embodiments, the method further comprises fractionating a whole blood sample of the subject to obtain the cell-free biological sample.

In some embodiments, the first assay comprises a cfRNA assay or a DNA methylation assay. In some embodiments, the DNA methylation assay comprises 5mC and or 5hmC detection assay. In some embodiments, the first assay or the second assay comprises quantitative polymerase chain reaction (qPCR). In some embodiments, the first assay or the second assay comprises a home use test configured to be performed in a home setting.

In some embodiments, the trained algorithm determines the presence or susceptibility of the pregnancy-related state of the subject at a sensitivity of at least about 80%. In some embodiments, the trained algorithm determines the presence or susceptibility of the pregnancy-related state of the subject at a sensitivity of at least about 90%. In some embodiments, the trained algorithm determines the presence or susceptibility of the pregnancy-related state of the subject at a sensitivity of at least about 95%.

In some embodiments, the trained algorithm determines the presence or susceptibility of the pregnancy-related state of the subject at a positive predictive value (PPV) of at least about 70%. In some embodiments, the trained algorithm determines the presence or susceptibility of the pregnancy-related state of the subject at a positive predictive value (PPV) of at least about 80%. In some embodiments, the trained algorithm determines the presence or susceptibility of the pregnancy-related state thereof of the subject at a positive predictive value (PPV) of at least about 90%.

In some embodiments, the trained algorithm determines the presence or susceptibility of the pregnancy-related state of the subject with an Area Under Curve (AUC) of at least about 0.90. In some embodiments, the trained algorithm determines the presence or susceptibility of the pregnancy-related state of the subject with an Area Under Curve (AUC) of at least about 0.95. In some embodiments, the trained algorithm determines the presence or susceptibility of the pregnancy-related state of the subject with an Area Under Curve (AUC) of at least about 0.99.

In some embodiments, the subject is asymptomatic for one or more of: pre-term birth, onset of labor, pregnancy-related hypertensive disorders, preeclampsia, eclampsia, gestational diabetes, a congenital disorder of a fetus of the subject, ectopic pregnancy, spontaneous abortion, stillbirth, post-partum complications, hyperemesis gravidarum (morning sickness), hemorrhage or excessive bleeding during delivery, premature rupture of membrane, premature rupture of membrane in pre-term birth, placenta previa (placenta covering the cervix), placenta accreta spectrum disorders (placenta increta, placenta percreta, and placenta accreta), intrauterine/fetal growth restriction, macrosomia (large fetus for gestational age), neonatal conditions, and abnormal fetal development stages or states. For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

In some embodiments, the cell-free biological sample is collected from the subject within a given gestational age interval for detection of a pregnancy-related state. In some embodiments, the given gestational age interval is within about 1 day, about 2 days, about 3 days, about 4 days, about 5 days, about 6 days about 7 days, about 8 days, about 9 days, about 10 days, about 11 days, about 12 days, about 13 days, about 14 days, about 3 weeks, or about 4 weeks from a given gestational age. In some embodiments, the given gestational age is about 0 weeks, about 1 week, about 2 weeks, about 3 weeks, about 4 weeks, about 5 weeks, about 6 weeks, about 7 weeks, about 8 weeks, about 9 weeks, about 10 weeks, about 11 week, about 12 weeks, about 13 weeks, about 14 weeks, about 15 weeks, about 16 weeks, about 17 weeks, about 18 weeks, about 19 weeks, about 20 weeks, about 21 week, about 22 weeks, about 23 weeks, about 24 weeks, about 25 weeks, about 26 weeks, about 27 weeks, about 28 weeks, about 29 weeks, about 30 weeks, about 31 week, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, or about 45 weeks. In some embodiments, the pregnancy-related state comprises one or more of: pre-term birth, onset of labor, pregnancy-related hypertensive disorders, preeclampsia, eclampsia, gestational diabetes, a congenital disorder of a fetus of the subject, ectopic pregnancy, spontaneous abortion, stillbirth, post-partum complications, hyperemesis gravidarum (morning sickness), hemorrhage or excessive bleeding during delivery, premature rupture of membrane, premature rupture of membrane in pre-term birth, placenta previa (placenta covering the cervix), placenta accreta spectrum disorders (placenta increta, placenta percreta, and placenta accreta), intrauterine/fetal growth restriction, macrosomia (large fetus for gestational age), neonatal conditions, and abnormal fetal development stages or states. For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

In some embodiments, the trained algorithm is trained using at least about 10 independent training samples associated with the presence or susceptibility of the pregnancy-related state. In some embodiments, the trained algorithm is trained using no more than about 100 independent training samples associated with the presence or susceptibility of the pregnancy-related state. In some embodiments, the trained algorithm is trained using a first set of independent training samples associated with a presence or susceptibility of the pregnancy-related state and a second set of independent training samples associated with an absence or no susceptibility of the pregnancy-related state. In some embodiments, the method further comprises using the trained algorithm to process a set of clinical health data of the subject to determine the presence or susceptibility of the pregnancy-related state.

In some embodiments, (a) comprises (i) subjecting the cell-free biological sample to conditions that are sufficient to isolate, enrich, or extract a set of ribonucleic (RNA) molecules, deoxyribonucleic acid (DNA) molecules, 5mC and/or 5hmC containing deoxyribonucleic acid (DNA), and (ii) analyzing the set of RNA molecules, DNA, 5mC and/or 5hmC DNA molecules, proteins, or metabolites using the first assay to generate the first dataset.

In some embodiments, the sequencing is massively parallel sequencing. In some embodiments, the sequencing comprises nucleic acid amplification. In some embodiments, the nucleic acid amplification comprises polymerase chain reaction (PCR). In some embodiments, the sequencing comprises use of simultaneous reverse transcription (RT) and polymerase chain reaction (PCR). In some embodiments, the method further comprises using probes configured to selectively enrich the set of nucleic acid molecules corresponding to a panel of one or more loci. In some embodiments, the probes are nucleic acid primers. In some embodiments, the probes have sequence complementarity with nucleic acid sequences of the panel of the one or more loci.

In some embodiments, the panel of the one or more loci comprises at least 5 distinct loci. In some embodiments, the panel of the one or more loci comprises at least 10 distinct loci.

In some embodiments, the set of biomarkers comprises a locus associated with gestational age, wherein the locus is selected from the group consisting of genes listed in Table 3, non-genic loci listed in Table 4, genes listed in Table 5, non-genic loci listed in Table 6, genes listed in Table 7, and genes listed in Table 9. In some embodiments, the panel of the one or more loci comprises a locus associated with preeclampsia, wherein the locus is selected from the group consisting of genomic and non-genomic or aggregated loci listed in Table 11, Table 12, Table 13, and the CLDN7, PAPPA2, SNORD14A, PLEKHH1, MAGEA10, TLE6, and FABP11 genes. In some embodiments, the panel of the one or more loci comprises at least 5 distinct loci. In some embodiments, the panel of the one or more loci comprises at least 10 distinct loci. In some embodiments, the panel of the one or more loci comprises at least 25 distinct loci. In some embodiments, the panel of the one or more loci comprises at least 50 distinct loci. In some embodiments, the panel of the one or more loci comprises at least 100 distinct loci. In some embodiments, the panel of the one or more loci comprises at least 150 distinct loci.

In some embodiments, the cell-free biological sample is processed without nucleic acid isolation, enrichment, or extraction.

In some embodiments, the report is presented on a graphical user interface of an electronic device of a user. In some embodiments, the user is the subject.

In some embodiments, the method further comprises determining a likelihood of the determination of the presence or susceptibility of the pregnancy-related state of the subject.

In some embodiments, the trained algorithm comprises a supervised machine learning algorithm. In some embodiments, the supervised machine learning algorithm comprises a deep learning algorithm, a support vector machine (SVM), a neural network, logistic regression, recursive feature elimination (RFE), or a Random Forest. In some embodiments, the trained algorithm comprises a differential expression algorithm. In some embodiments, the differential expression algorithm comprises a use comparison of stochastic models, generalized Poisson (GPseq), mixed Poisson (TSPM), Poisson log-linear (PoissonSeq), negative binomial (edgeR, DESeq, baySeq, NBPSeq), linear model fit by MAANOVA, Spearman correlation, or a combination thereof.

In some embodiments, the method further comprises providing the subject with a therapeutic intervention for the presence or susceptibility of the pregnancy-related state. In some embodiments, the therapeutic intervention comprises hydroxyprogesterone caproate, a vaginal progesterone, a natural progesterone IVR product, an prostaglandin F2 alpha receptor antagonist, or a beta2-adrenergic receptor agonist.

In some embodiments, the method further comprises monitoring the presence or susceptibility of the pregnancy-related state, wherein the monitoring comprises assessing the presence or susceptibility of the pregnancy-related state of the subject at a plurality of time points, wherein the assessing is based at least on the presence or susceptibility of the pregnancy-related state determined in (d) at each of the plurality of time points.

In some embodiments, a difference in the assessment of the presence or susceptibility of the pregnancy-related state of the subject among the plurality of time points is indicative of one or more clinical indications selected from the group consisting of: (i) a diagnosis of the presence or susceptibility of the pregnancy-related state of the subject, (ii) a prognosis of the presence or susceptibility of the pregnancy-related state of the subject, and (iii) an efficacy or non-efficacy of a course of treatment for treating the presence or susceptibility of the pregnancy-related state of the subject.

In some embodiments, the method further comprises stratifying the pre-term birth by using the trained algorithm to determine a molecular sub-type of the pre-term birth from among a plurality of distinct molecular subtypes of pre-term birth. In some embodiments, the plurality of distinct molecular subtypes of pre-term birth comprises a molecular subtype of pre-term birth selected from the group consisting of presence or history of prior pre-term birth, presence or history of spontaneous pre-term birth, presence or history of late miscarriage, presence or history of receiving cervical surgery, presence or history of a uterine anomaly, presence or history of ethnicity specific pre-term birth risk (e.g., among an African-American population), and presence or history of pre-term premature rupture of membrane (PPROM).

In some embodiments, the method further comprises stratifying the preeclampsia by using the trained algorithm to determine a molecular sub-type of the preeclampsia from among a plurality of distinct molecular subtypes of preeclampsia comprise a molecular subtype of preeclampsia selected from the group consisting of history of chronic/pre-existing hypertension, gestational hypertension, mild preeclampsia (with delivery >34 weeks), severe preeclampsia (with delivery <34 weeks), eclampsia, HELLP syndrome.

In some embodiments, (a) comprises processing a first cell-free biological sample derived from the subject at a first time point, and (b) comprises processing a second cell-free biological sample derived from the subject at a second time point which is before the first time point. In some embodiments, (a) comprises processing a first cell-free biological sample derived from the subject at a first time point, and (b) comprises processing a second cell-free biological sample derived from the subject at a second time point which is after the first time point. In some embodiments, (a) comprises processing a first cell-free biological sample derived from the subject at a first time point, and (b) comprises processing a second cell-free biological sample derived from the subject at a second time point which is after the first time point

In another aspect, the present disclosure provides a computer-implemented method for predicting a risk of pre-term birth of a subject, comprising: (a) receiving clinical health data of the subject, wherein the clinical health data comprises a plurality of quantitative or categorical measures of the subject; (b) using an algorithm (e.g., a trained algorithm) to process the clinical health data of the subject to determine a risk score indicative of the risk of pre-term birth of the subject; and (c) electronically outputting a report indicative of the risk score indicative of the risk of pre-term birth of the subject.

In another aspect, the present disclosure provides a computer-implemented method for predicting a risk of preeclampsia of a subject, comprising: (a) receiving clinical health data of the subject, wherein the clinical health data comprises a plurality of quantitative or categorical measures of the subject; (b) using an algorithm (e.g., a trained algorithm) to process the clinical health data of the subject to determine a risk score indicative of the risk of preeclampsia of the subject; and (c) electronically outputting a report indicative of the risk score indicative of the risk of preeclampsia of the subject.

In another aspect, the present disclosure provides a computer-implemented method for predicting a risk for a pregnancy-related state of a maternal, newborn, infant, or offspring subject, comprising: (a) receiving clinical health data of the subject, wherein the clinical health data comprises a plurality of quantitative or categorical measures of the subject; (b) using an algorithm (e.g., a trained algorithm) to process the clinical health data of the subject to determine a risk score indicative of the risk of pre-term birth of the subject; and (c) electronically outputting a report indicative of the risk score indicative of the risk of the pregnancy-related state of the maternal, newborn, infant, or offspring subject.

In some embodiments, the pregnancy-related states are indicative of an impact to postpartum health (e.g., short-term or long-term) of a maternal, newborn, infant, or offspring subject. In some embodiments, pregnancy-related states of a maternal subject comprise postpartum health conditions that develop later (e.g., 5 to 15 years postpartum) such as hypertension, diabetes, and cardiovascular diseases. In some embodiments, health-related conditions of a newborn, infant, or offspring subject comprise health conditions that develop later (e.g., 5 to 15 years postpartum) such as hypertension, diabetes, cardiovascular diseases, and neurological development.

In some embodiments, the clinical health data comprises one or more quantitative measures selected from the group consisting of age, weight, height, body mass index (BMI), blood pressure, heart rate, glucose levels, number of previous pregnancies, and number of previous births. In some embodiments, the clinical health data comprises one or more categorical measures selected from the group consisting of race, ethnicity, history of medication or other clinical treatment, history of tobacco use, history of alcohol consumption, daily activity or fitness level, genetic test results, blood test results, imaging results, and fetal screening results.

In some embodiments, the trained algorithm determines the risk of pre-term birth of the subject at a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the trained algorithm determines the risk of pre-term birth of the subject at a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the trained algorithm determines the risk of pre-term birth of the subject at a positive predictive value (PPV) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the trained algorithm determines the risk of pre-term birth of the subject at a negative predictive value (NPV) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the trained algorithm determines the risk of pre-term birth of the subject with an Area Under Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, or at least about 0.99.

In some embodiments, the trained algorithm determines the risk of preeclampsia of the subject at a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the trained algorithm determines the risk of preeclampsia of the subject at a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the trained algorithm determines the risk of preeclampsia of the subject at a positive predictive value (PPV) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the trained algorithm determines the risk of preeclampsia of the subject at a negative predictive value (NPV) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the trained algorithm determines the risk of preeclampsia of the subject with an Area Under Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, or at least about 0.99.

In some embodiments, the subject is asymptomatic for one or more of: pre-term birth, onset of labor, pregnancy-related hypertensive disorders, preeclampsia, eclampsia, gestational diabetes, a congenital disorder of a fetus of the subject, ectopic pregnancy, spontaneous abortion, stillbirth, post-partum complications, hyperemesis gravidarum (morning sickness), hemorrhage or excessive bleeding during delivery, premature rupture of membrane, premature rupture of membrane in pre-term birth, placenta previa (placenta covering the cervix), placenta accreta spectrum disorders (placenta increta, placenta percreta, and placenta accreta), intrauterine/fetal growth restriction, macrosomia (large fetus for gestational age), neonatal conditions, and abnormal fetal development stages or states. For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

In some embodiments, the trained algorithm is trained using at least about 10 independent training samples associated with pre-term birth. In some embodiments, the trained algorithm is trained using no more than about 100 independent training samples associated with pre-term birth. In some embodiments, the trained algorithm is trained using a first set of independent training samples associated with a presence of pre-term birth and a second set of independent training samples associated with an absence of pre-term birth.

In some embodiments, the trained algorithm is trained using at least about 10 independent training samples associated with preeclampsia. In some embodiments, the trained algorithm is trained using no more than about 100 independent training samples associated with preeclampsia In some embodiments, the trained algorithm is trained using a first set of independent training samples associated with a presence of preeclampsia and a second set of independent training samples associated with an absence of preeclampsia.

In some embodiments, the report is presented on a graphical user interface of an electronic device of a user. In some embodiments, the user is the subject.

In some embodiments, the trained algorithm comprises a supervised machine learning algorithm. In some embodiments, the supervised machine learning algorithm comprises a deep learning algorithm, a support vector machine (SVM), a neural network, logistic regression, recursive feature elimination (RFE), or a Random Forest. In some embodiments, the trained algorithm comprises a differential expression algorithm. In some embodiments, the differential expression algorithm comprises a use comparison of stochastic models, generalized Poisson (GPseq), mixed Poisson (TSPM), Poisson log-linear (PoissonSeq), negative binomial (edgeR, DESeq, baySeq, NBPSeq), linear model fit by MAANOVA, Spearman correlation, or a combination thereof.

In some embodiments, the method further comprises providing the subject with a therapeutic intervention based at least in part on the risk score indicative of the risk of pre-term birth. In some embodiments, the therapeutic intervention comprises hydroxyprogesterone caproate, a vaginal progesterone, a natural progesterone IVR product, an prostaglandin F2 alpha receptor antagonist, or a beta2-adrenergic receptor agonist.

In some embodiments, the method further comprises providing the subject with a therapeutic intervention based at least in part on the risk score indicative of the risk of preeclampsia. In some embodiments, the therapeutic intervention comprises antihypertensive drug therapy (such as but not limited to hydralazine, labetalol, nifedipine, and sodium nitroprusside), management or prevention of seizures (such as but not limited to magnesium sulfate, phenytoin, and diazepam), or prevention by low-dose aspirin therapy (e.g., 100 mg per day or less) to reduce the incidence of preeclampsia.

In some embodiments, the method further comprises monitoring the risk of pre-term birth, wherein the monitoring comprises assessing the risk of pre-term birth of the subject at a plurality of time points, wherein the assessing is based at least on the risk score indicative of the risk of pre-term birth determined in (b) at each of the plurality of time points.

In some embodiments, the method further comprises monitoring the risk of preeclampsia, wherein the monitoring comprises assessing the risk of preeclampsia of the subject at a plurality of time points, wherein the assessing is based at least on the risk score indicative of the risk of preeclampsia determined in (b) at each of the plurality of time points.

In some embodiments, the method further comprises refining the risk score indicative of the risk of pre-term birth of the subject by performing one or more subsequent clinical tests for the subject, and processing results from the one or more subsequent clinical tests using a trained algorithm to determine an updated risk score indicative of the risk of pre-term birth of the subject. In some embodiments, the one or more subsequent clinical tests comprise an ultrasound imaging or a blood test. In some embodiments, the risk score comprises a likelihood of the subject having a pre-term birth within a pre-determined duration of time.

In some embodiments, the method further comprises refining the risk score indicative of the risk of preeclampsia of the subject by performing one or more subsequent clinical tests for the subject, and processing results from the one or more subsequent clinical tests using a trained algorithm to determine an updated risk score indicative of the risk of preeclampsia of the subject. In some embodiments, the one or more subsequent clinical tests comprise an ultrasound imaging or a blood test. In some embodiments, the risk score comprises a likelihood of the subject having a preeclampsia within a pre-determined duration of time.

In some embodiments, the pre-determined duration of time is about 1 hour, about 2 hours, about 4 hours, about 6 hours, about 8 hours, about 10 hours, about 12 hours, about 14 hours, about 16 hours, about 18 hours, about 20 hours, about 22 hours, about 24 hours, about 1.5 days, about 2 days, about 2.5 days, about 3 days, about 3.5 days, about 4 days, about 4.5 days, about 5 days, about 5.5 days, about 6 days, about 6.5 days, about 7 days, about 8 days, about 9 days, about 10 days, about 12 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 13 weeks, or more than about 13 weeks.

In another aspect, the present disclosure provides a computer system for predicting a risk of pre-term birth of a subject, comprising: a database that is configured to store clinical health data of the subject, wherein the clinical health data comprises a plurality of quantitative or categorical measures of the subject; and one or more computer processors operatively coupled to the database, wherein the one or more computer processors are individually or collectively programmed to: (i) use an algorithm (e.g., a trained algorithm) to process the clinical health data of the subject to determine a risk score indicative of the risk of pre-term birth of the subject; and (ii) electronically output a report indicative of the risk score indicative of the risk of pre-term birth of the subject.

In another aspect, the present disclosure provides a computer system for predicting a risk of preeclampsia of a subject, comprising: a database that is configured to store clinical health data of the subject, wherein the clinical health data comprises a plurality of quantitative or categorical measures of the subject; and one or more computer processors operatively coupled to the database, wherein the one or more computer processors are individually or collectively programmed to: (i) use an algorithm (e.g., a trained algorithm) to process the clinical health data of the subject to determine a risk score indicative of the risk of preeclampsia of the subject; and (ii) electronically output a report indicative of the risk score indicative of the risk of preeclampsia of the subject.

In some embodiments, the computer system further comprises an electronic display operatively coupled to the one or more computer processors, wherein the electronic display comprises a graphical user interface that is configured to display the report.

In another aspect, the present disclosure provides a non-transitory computer readable medium comprising machine-executable code that, upon execution by one or more computer processors, implements a method for predicting a risk of pre-term birth of a subject, the method comprising: (a) receiving clinical health data of the subject, wherein the clinical health data comprises a plurality of quantitative or categorical measures of the subject; (b) using an algorithm (e.g., a trained algorithm) to process the clinical health data of the subject to determine a risk score indicative of the risk of pre-term birth of the subject; and (c) electronically outputting a report indicative of the risk score indicative of the risk of pre-term birth of the subject.

In another aspect, the present disclosure provides a non-transitory computer readable medium comprising machine-executable code that, upon execution by one or more computer processors, implements a method for predicting a risk of preeclampsia of a subject, the method comprising: (a) receiving clinical health data of the subject, wherein the clinical health data comprises a plurality of quantitative or categorical measures of the subject; (b) using an algorithm (e.g., a trained algorithm) to process the clinical health data of the subject to determine a risk score indicative of the risk of preeclampsia of the subject; and (c) electronically outputting a report indicative of the risk score indicative of the risk of preeclampsia of the subject.

In some embodiments, the set of biomarkers comprises at least 5 distinct loci. In some embodiments, the set of biomarkers comprises at least 10 distinct loci. In some embodiments, the set of biomarkers comprises at least 25 distinct loci. In some embodiments, the set of biomarkers comprises at least 50 distinct loci. In some embodiments, the set of biomarkers comprises at least 100 distinct loci. In some embodiments, the set of biomarkers comprises at least 150 distinct loci.

In some embodiments, the clinical intervention is selected from a plurality of clinical interventions. In some embodiments, the method further comprises determining a likelihood of the determination of the susceptibility of the pregnancy-related state of the subject, after which subject may be provided with the clinical intervention. In some embodiments, the clinical intervention comprises a pharmacological, surgical, or procedural treatment to reduce severity, delay, or eliminate the future susceptibility pregnancy-related state of the subject (e.g., aspirin for PE and steroids for PTB).

In another aspect, the present disclosure provides a method for identifying or monitoring a presence or susceptibility of a pregnancy-related state of a subject, comprising: (a) using a first assay to process a first cell-free biological sample derived from the subject to generate a first dataset; (b) based at least in part on the first dataset generated in (a), using a second assay different from the first assay to process a second cell-free biological sample derived from the subject to generate a second dataset indicative of the presence or susceptibility of the pregnancy-related state at a specificity greater than the first dataset; (c) using a trained algorithm to process at least the second dataset to determine the presence or susceptibility of the pregnancy-related state, which trained algorithm has an accuracy of at least about 80% over 50 independent samples; and (d) electronically outputting a report indicative of the presence or susceptibility of the pregnancy-related state of the subject.

In some embodiments, the first assay comprises using cell-free ribonucleic acid (cfRNA) molecules derived from the first cell-free biological sample to generate transcriptomic data, and using cell-free deoxyribonucleic acid (cfDNA) molecules derived from the first cell-free biological sample to generate DNA methylation status and/or genomic data. In some embodiments, the first cell-free biological sample is from a blood of the subject. In some embodiments, the first cell-free biological sample is from a urine of the subject. In some embodiments, the first dataset comprises a first set of biomarkers associated with the pregnancy-related state. In some embodiments, the second dataset comprises a second set of biomarkers associated with the pregnancy-related state. In some embodiments, the second set of biomarkers is different from the first set of biomarkers.

In some embodiments, the pregnancy-related state is selected from the group consisting of pre-term birth, full-term birth, gestational age, due date, onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), placenta accreta spectrum disorders (placenta increta, placenta percreta, and placenta accreta), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and fetal development stages or states (e.g., normal fetal organ function or development, and abnormal fetal organ function or development). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus. In some embodiments, the pregnancy-related state comprises pre-term birth. In some embodiments, the pregnancy-related state comprises gestational age.

In some embodiments, the cell-free biological sample is selected from the group consisting of cell-free ribonucleic acid (cfRNA), cell-free deoxyribonucleic acid (cfDNA), cell-free fetal DNA (cffDNA), plasma, serum, urine, saliva, amniotic fluid, and derivatives thereof. In some embodiments, the first cell-free biological sample or the second cell-free biological sample is obtained or derived from the subject using an ethylenediaminetetraacetic acid (EDTA) collection tube, a cell-free RNA collection tube, or a cell-free DNA collection tube. In some embodiments, the method further comprises fractionating a whole blood sample of the subject to obtain the first cell-free biological sample or the second cell-free biological sample. In some embodiments, (i) the first assay comprises a cfRNA assay and the second assay comprises a cfDNA methylation assay, or (ii) the first assay comprises a cfDNA methylation assay and the second assay comprises a cfRNA assay. In some embodiments, (i) the first cell-free biological sample comprises cfRNA and the second cell-free biological sample comprises urine, or (ii) the first cell-free biological sample comprises urine and the second cell-free biological sample comprises cfRNA. In some embodiments, the first assay or the second assay comprises quantitative polymerase chain reaction (qPCR). In some embodiments, the first assay or the second assay comprises a home use test configured to be performed in a home setting.

In some embodiments, the first dataset is indicative of the presence or susceptibility of the pregnancy-related state at a sensitivity of at least about 80%. In some embodiments, the first dataset is indicative of the presence or susceptibility of the pregnancy-related state at a sensitivity of at least about 90%. In some embodiments, the first dataset is indicative of the presence or susceptibility of the pregnancy-related state at a sensitivity of at least about 95%. In some embodiments, the first dataset is indicative of the presence or susceptibility of the pregnancy-related state at a positive predictive value (PPV) of at least about 70%. In some embodiments, the first dataset is indicative of the presence or susceptibility of the pregnancy-related state at a positive predictive value (PPV) of at least about 80%. In some embodiments, the first dataset is indicative of the presence or susceptibility of the pregnancy-related state at a positive predictive value (PPV) of at least about 90%. In some embodiments, the second dataset is indicative of the presence or susceptibility of the pregnancy-related state at a specificity of at least about 90%. In some embodiments, the second dataset is indicative of the presence or susceptibility of the pregnancy-related state at a specificity of at least about 95%. In some embodiments, the second dataset is indicative of the presence or susceptibility of the pregnancy-related state at a specificity of at least about 99%. In some embodiments, the second dataset is indicative of the presence or susceptibility of the pregnancy-related state at a negative predictive value (NPV) of at least about 90%. In some embodiments, the second dataset is indicative of the presence or susceptibility of the pregnancy-related state at a negative predictive value (NPV) of at least about 95%. In some embodiments, the second dataset is indicative of the presence or susceptibility of the pregnancy-related state at a negative predictive value (NPV) of at least about 99%. In some embodiments, the trained algorithm determines the presence or susceptibility of the pregnancy-related state of the subject with an Area Under Curve (AUC) of at least about 0.90. In some embodiments, the trained algorithm determines the presence or susceptibility of the pregnancy-related state of the subject with an Area Under Curve (AUC) of at least about 0.95. In some embodiments, the trained algorithm determines the presence or susceptibility of the pregnancy-related state of the subject with an Area Under Curve (AUC) of at least about 0.99.

In some embodiments, the subject is asymptomatic for one or more of: pre-term birth, onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), placenta accreta spectrum disorders (placenta increta, placenta percreta, and placenta accreta), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and abnormal fetal development stages or states (e.g., abnormal fetal organ function or development). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

In some embodiments, the trained algorithm is trained using at least about 10 independent training samples associated with the pregnancy-related state. In some embodiments, the trained algorithm is trained using no more than about 100 independent training samples associated with the pregnancy-related state. In some embodiments, the trained algorithm is trained using a first set of independent training samples associated with a presence of the pregnancy-related state and a second set of independent training samples associated with an absence of the pregnancy-related state. In some embodiments, the method further comprises using the trained algorithm to process the first dataset to determine the presence or susceptibility of the pregnancy-related state. In some embodiments, the method further comprises using the trained algorithm to process a set of clinical health data of the subject to determine the presence or susceptibility of the pregnancy-related state.

In some embodiments, (a) comprises (i) subjecting the first cell-free biological sample to conditions that are sufficient to isolate, enrich, or extract a first set of ribonucleic acid (RNA) molecules and (ii) analyzing the first set of RNA molecules using the first assay to generate the first dataset. In some embodiments, the method further comprises extracting a first set of RNA molecules from the first cell-free biological sample, and subjecting the first set of nucleic acid molecules to sequencing to generate a first set of sequencing reads, wherein the first dataset comprises the first set of sequencing reads. In some embodiments, the method further comprises extracting a first set of DNA molecules from the first cell-free biological sample, and assaying the first set of DNA methylation status to generate the first dataset In some embodiments, (b) comprises (i) subjecting the second cell-free biological sample to conditions that are sufficient to isolate, enrich, or extract a second set of ribonucleic acid (RNA) molecules and (ii) analyzing the second set of RNA molecules using the second assay to generate the second dataset. In some embodiments, the method further comprises extracting a second set of nucleic acid molecules from the second cell-free biological sample, and subjecting the second set of nucleic acid molecules to sequencing to generate a second set of sequencing reads, wherein the second dataset comprises the second set of sequencing reads. In some embodiments, the sequencing is massively parallel sequencing. In some embodiments, the sequencing comprises nucleic acid amplification. In some embodiments, the nucleic acid amplification comprises polymerase chain reaction (PCR). In some embodiments, the sequencing comprises use of simultaneous reverse transcription (RT) and polymerase chain reaction (PCR).

In some embodiments, the method further comprises using probes configured to selectively enrich the first set of nucleic acid molecules or the second set of nucleic acid molecules corresponding to a panel of one or more loci. In some embodiments, the probes are nucleic acid primers. In some embodiments, the probes have sequence complementarity with nucleic acid sequences of the panel of the one or more loci.

In some embodiments, the set of biomarkers comprises a locus associated with gestational age, wherein the locus is selected from the group consisting of genes listed in Table 3, non-genic loci listed in Table 4, genes listed in Table 5, non-genic loci listed in Table 6, genes listed in Table 7, and genes listed in able 9. In some embodiments, the panel of the one or more loci comprises a locus associated with preeclampsia, wherein the locus is selected from the group consisting of genomic and non-genomic or aggregated loci listed in Table 11, Table 12, Table 13, and the CLDN7, PAPPA2, SNORD14A, PLEKHH1, MAGEA10, TLE6, and FABP11 genes.

In some embodiments, the panel of the one or more loci comprises at least 5 distinct loci. In some embodiments, the panel of the one or more loci comprises at least 10 distinct loci. In some embodiments, the panel of the one or more loci comprises at least 25 distinct loci. In some embodiments, the panel of the one or more loci comprises at least 50 distinct loci. In some embodiments, the panel of the one or more loci comprises at least 100 distinct loci. In some embodiments, the panel of the one or more loci comprises at least 150 distinct loci. In some embodiments, the first cell-free biological sample or the second cell-free biological sample is processed without nucleic acid isolation, enrichment, or extraction. In some embodiments, the report is presented on a graphical user interface of an electronic device of a user. In some embodiments, the user is the subject.

In some embodiments, the method further comprises determining a likelihood of the determination of the presence or susceptibility of the pregnancy-related state of the subject. In some embodiments, the trained algorithm comprises a supervised machine learning algorithm. In some embodiments, the supervised machine learning algorithm comprises a deep learning algorithm, a support vector machine (SVM), a neural network, logistic regression, recursive feature elimination (RFE), or a Random Forest. In some embodiments, the trained algorithm comprises a differential expression algorithm. In some embodiments, the differential expression algorithm comprises a use comparison of stochastic models, generalized Poisson (GPseq), mixed Poisson (TSPM), Poisson log-linear (PoissonSeq), negative binomial (edgeR, DESeq, baySeq, NBPSeq), linear model fit by MAANOVA, Spearman correlation, or a combination thereof. In some embodiments, the method further comprises providing the subject with a therapeutic intervention for the presence or susceptibility of the pregnancy-related state. In some embodiments, therapeutic intervention comprises a progesterone treatment such as hydroxyprogesterone caproate (e.g., 17-alpha hydroxyprogesterone caproate (17-P), LPCN 1107 from Lipocine, Makena from AMAG Pharma), a vaginal progesterone, or a natural progesterone IVR product (e.g., DARE-FRT1 (JNP-0301) from Juniper Pharma); a prostaglandin F2 alpha receptor antagonist (e.g., OBE022 from ObsEva); or a beta2-adrenergic receptor agonist (e.g., bedoradrine sulfate (MN-221) from MediciNova). Therapeutic interventions may be described by, for example, “WHO Recommendations on Interventions to Improve Preterm Birth Outcomes,” World Health Organization, 2015, which is hereby incorporated by reference in its entirety. In some embodiments, the method further comprises monitoring the presence or susceptibility of the pregnancy-related state, wherein the monitoring comprises assessing the presence or susceptibility of the pregnancy-related state of the subject at a plurality of time points, wherein the assessing is based at least on the presence or susceptibility of the pregnancy-related state determined in (d) at each of the plurality of time points. In some embodiments, a difference in the assessment of the presence or susceptibility of the pregnancy-related state of the subject among the plurality of time points is indicative of one or more clinical indications selected from the group consisting of: (i) a diagnosis of the presence or susceptibility of the pregnancy-related state of the subject, (ii) a prognosis of the presence or susceptibility of the pregnancy-related state of the subject, and (iii) an efficacy or non-efficacy of a course of treatment for treating the presence or susceptibility of the pregnancy-related state of the subject.

In some embodiments, the method further comprises stratifying the pre-term birth by using the trained algorithm to determine a molecular sub-type of the pre-term birth from among a plurality of distinct molecular subtypes of pre-term birth. In some embodiments, the plurality of distinct molecular subtypes of pre-term birth comprises a molecular subtype of pre-term birth selected from the group consisting of presence or history of prior pre-term birth, presence or history of spontaneous pre-term birth, presence or history of late miscarriage, presence or history of receiving cervical surgery, presence or history of a uterine anomaly, presence or history of ethnicity specific pre-term birth risk (e.g., among an African-American population), and presence or history of pre-term premature rupture of membrane (PPROM).

In some embodiments, the method further comprises stratifying the preeclampsia by using the trained algorithm to determine a molecular sub-type of the preeclampsia from among a plurality of distinct molecular subtypes of preeclampsia. In some embodiments, the plurality of distinct molecular subtypes of preeclampsia comprises a molecular subtype of preeclampsia selected from the group consisting of: presence or history of chronic or pre-existing hypertension, presence or history of gestational hypertension, presence or history of mild preeclampsia (e.g., with delivery greater than 34 weeks gestational age), presence or history of severe preeclampsia (with delivery less than 34 weeks gestational age), presence or history of eclampsia, and presence or history of HELLP syndrome.

In another aspect, the present disclosure provides a computer system for identifying or monitoring a presence or susceptibility of the pregnancy-related state of a subject, comprising: a database that is configured to store a first dataset and a second dataset, wherein the second dataset is indicative of the presence or susceptibility of the pregnancy-related state at a specificity greater than the first dataset; and one or more computer processors operatively coupled to the database, wherein the one or more computer processors are individually or collectively programmed to: (i) use a trained algorithm to process at least the second dataset to determine the presence or susceptibility of the pregnancy-related state, which trained algorithm has an accuracy of at least about 80% over 50 independent samples; and (ii) electronically output a report indicative of the presence or susceptibility of the pregnancy-related state of the subject.

In some embodiments, the computer system further comprises an electronic display operatively coupled to the one or more computer processors, wherein the electronic display comprises a graphical user interface that is configured to display the report.

In another aspect, the present disclosure provides a non-transitory computer readable medium comprising machine-executable code that, upon execution by one or more computer processors, implements a method for identifying or monitoring a presence or susceptibility of the pregnancy-related state of a subject, the method comprising: (a) obtaining a first dataset, and a second dataset, wherein the second dataset is indicative of the presence or susceptibility of the pregnancy-related state at a specificity greater than the first dataset; (b) using a trained algorithm to process at least the second dataset to determine the pregnancy-related state, which trained algorithm has an accuracy of at least about 80% over 50 independent samples; and (c) electronically outputting a report indicative of the presence or susceptibility of the pregnancy-related state of the subject.

In another aspect, the present disclosure provides a method for identifying a presence or susceptibility of pregnancy-related state of a subject, comprising (i) assaying a first cell-free biological sample derived from the subject with a first assay to generate a first dataset, (ii) assaying a second cell-free biological sample derived from the subject with a second assay to generate a second dataset that is indicative of the presence or susceptibility of the pregnancy-related state at a specificity greater than the first dataset, and (iii) using a trained algorithm to process at least the second dataset to determine the presence or susceptibility of the pregnancy-related state at an accuracy of at least about 80%. In some embodiments, the accuracy is at least about 90%. In some embodiments, the pregnancy-related state is selected from the group consisting of pre-term birth, full-term birth, gestational age, due date, onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), placenta accreta spectrum disorders (placenta increta, placenta percreta, and placenta accreta), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and fetal development stages or states (e.g., normal fetal organ function or development, and abnormal fetal organ function or development). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

In another aspect, the present disclosure provides a method for determining that a subject is at risk of pre-term birth, comprising assaying a cell-free biological sample derived from the subject to generate a dataset that is indicative of the pre-term birth risk at a specificity of at least 80%, and using a trained algorithm that is trained on samples independent of the cell-free biological sample to determine that the subject is at risk of pre-term birth at an accuracy of at least about 80%. In some embodiments, the accuracy is at least about 90%.

In another aspect, the present disclosure provides a method for determining that a subject is at risk of preeclampsia, comprising assaying a cell-free biological sample derived from the subject to generate a dataset that is indicative of the preeclampsia risk at a specificity of at least 80%, and using a trained algorithm that is trained on samples independent of the cell-free biological sample to determine that the subject is at risk of preeclampsia at an accuracy of at least about 80%. In some embodiments, the accuracy is at least about 90%.

In another aspect, the present disclosure provides a method for detecting at least two health or physiological conditions of a fetus of a pregnant subject or of the pregnant subject, comprising: assaying a first cell-free biological sample obtained or derived from the pregnant subject at a first time point and a second cell-free biological sample obtained or derived from the pregnant subject at a second time point, to detect a first set of biomarkers at the first time point and a second set of biomarkers at the second time point, and analyzing the first set of biomarkers or the second set of biomarkers with a trained algorithm to detect the at least two health or physiological conditions.

In some embodiments, the at least two health or physiological conditions are selected from the group consisting of pre-term birth, full-term birth, gestational age, due date, onset of labor, a pregnancy-related hypertensive disorder, eclampsia, gestational diabetes, a congenital disorder of a fetus of the subject, ectopic pregnancy, spontaneous abortion, stillbirth, a post-partum complication, hyperemesis gravidarum, hemorrhage or excessive bleeding during delivery, premature rupture of membrane, premature rupture of membrane in pre-term birth, placenta previa, intrauterine/fetal growth restriction, macrosomia, a neonatal condition, and a fetal development stage or state. In some embodiments, the set of biomarkers comprises a locus associated with gestational age, wherein the locus is selected from the group consisting of genes listed in Table 3, non-genic loci listed in Table 4, genes listed in Table 5, non-genic loci listed in Table 6, genes listed in Table 7, and genes listed in Table 9. In some embodiments, the panel of the one or more loci comprises a locus associated with preeclampsia, wherein the locus is selected from the group consisting of group consisting of genomic and non-genomic or aggregated loci listed in Table 11, Table 12, Table 13, and the CLDN7, PAPPA2, SNORD14A, PLEKHH1, MAGEA10, TLE6, and FABP11 genes.

In another aspect, the present disclosure provides a method comprising: assaying one or more cell-free biological samples obtained or derived from a pregnant subject to detect a set of biomarkers; and analyzing the set of biomarkers to identify (1) a due date or a range thereof of a fetus of the pregnant subject and (2) a health or physiological condition of the fetus of the pregnant subject or of the pregnant subject.

In some embodiments, the method further comprises analyzing the set of biomarkers with a trained algorithm. In some embodiments, the health or physiological condition is selected from the group consisting of pre-term birth, full-term birth, gestational age, due date, onset of labor, a pregnancy-related hypertensive disorder, eclampsia, gestational diabetes, a congenital disorder of a fetus of the subject, ectopic pregnancy, spontaneous abortion, stillbirth, a post-partum complication, hyperemesis gravidarum, hemorrhage or excessive bleeding during delivery, premature rupture of membrane, premature rupture of membrane in pre-term birth, placenta previa, intrauterine/fetal growth restriction, macrosomia, a neonatal condition, and a fetal development stage or state. In some embodiments, the set of biomarkers comprises a locus associated with due date, wherein the locus is selected from the group consisting of genes listed in Table 3, non-genic loci listed in Table 4, genes listed in Table 5, non-genic loci listed in Table 6, genes listed in Table 7, and genes listed in Table 9. In some embodiments, the panel of the one or more loci comprises a locus associated with preeclampsia, wherein the locus is selected from the group consisting of group consisting of genomic and non-genomic or aggregated loci listed in Table 11, Table 12, Table 13, and the CLDN7, PAPPA2, SNORD14A, PLEKHH1, MAGEA10, TLE6, and FABP11 genes.

In some embodiments, the set of biomarkers comprises at least 5 distinct loci. In some embodiments, the panel of the one or more loci comprises a locus associated with preeclampsia, wherein the locus is selected from the group consisting of genomic and non-genomic or aggregated loci listed in Table 11, Table 12, Table 13, and the CLDN7, PAPPA2, SNORD14A, PLEKHH1, MAGEA10, TLE6, and FABP1 genes.

In another aspect, the present disclosure provides a method comprising: assaying one or more cell-free biological samples obtained or derived from a pregnant subject to detect a set of nucleic acids of non-human origin; and analyzing the set of nucleic acids of non-human origin to detect a health or physiological condition of a fetus of the pregnant subject or of the pregnant subject. In some embodiments, the nucleic acids of non-human origin comprise DNA or RNA of a non-human organism. In some embodiments, the non-human organism is a bacteria, a virus, or a parasite. In some embodiments, the method further comprises analyzing the set of nucleic acids of non-human origin using a trained algorithm.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an example workflow of a method for identifying or monitoring a pregnancy-related state of a subject.

FIG. 2 illustrates a computer system that is programmed or otherwise configured to implement methods provided herein.

FIG. 3 shows a distribution of collected blood samples in a gestational age cohort based on each participant's estimated gestational age and trimester at the time of collection of each blood sample.

FIG. 4A shows complete separation between samples from 13 non-pregnant subjects and 24 samples from pregnant subjects in the first trimester (gestational age <15 weeks), based on 5hmC DNA methylation profiling using the ADAM18 gene.

FIG. 4B shows a representative upward trend indicative of 5hmC increase in DNA methylation state for the LGR5 gene locus (left), and a downward trend indicative of 5hmC decrease in DNA methylation state for the TOX2 gene locus (right).

FIG. 5A shows signal separation based on detection of the PAPPA gene for a 5hmC DNA methylation state change compared to the PAPPA gene RNA expression change across the first, second, and third trimesters of pregnancy.

FIG. 5B shows even higher separation for 5hmC DNA methylation change for sum of top 10 GA genes compared to RNA expression.

FIG. 5C shows representative RNA and 5hmC DNA methylation signals and p-values for SVREP1 gene for separation pregnancy samples between the first and second trimester.

FIG. 6A shows signal separation based on detection of the PAPPA2 gene for a 5hmC DNA methylation state change compared to PAPPA2 gene RNA expression change across the first, second, and third trimesters of pregnancy.

FIG. 6B shows signal separation for detection of the MAGEA10 gene for a 5hmC DNA methylation state change compared to MAGEA10 gene RNA expression change across the first, second, and third trimesters of pregnancy.

FIG. 6C shows signal separation for detection of the TLE6 gene for a 5hmC DNA methylation state change compared to TLE6 gene RNA expression change across the first, second, and third trimesters of pregnancy.

FIG. 6D shows signal separation for detection of the PLEKHH1 gene for a 5hmC DNA methylation state change compared to PLEKHH1 gene RNA expression change across the first, second, and third trimesters of pregnancy.

FIG. 6E shows signal separation for detection of the FABP1 gene for a 5hmC DNA methylation state change compared to FABP1 gene RNA expression change across the first, second, and third trimesters of pregnancy.

FIG. 7 shows a distribution of collected samples for analysis of gestational ages at delivery for preeclampsia controls and cases, relative to the gestational ages.

FIG. 8 shows a QQ plot of P-values of the T-tests for each of a set of 5′-UTR features in the comparison of all preeclampsia cases (n=45) and all controls (n=44).

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an”, and “the” include plural references unless the context clearly dictates otherwise. For example, the term “a nucleic acid” includes a plurality of nucleic acids, including mixtures thereof.

As used herein, the term “subject,” generally refers to an entity or a medium that has testable or detectable genetic information. A subject may be a person, individual, or patient. A subject may be a vertebrate, such as, for example, a mammal. Non-limiting examples of mammals include humans, simians, farm animals, sport animals, rodents, and pets. A subject may be a pregnant female subject. The subject may be a woman having a fetus (or multiple fetuses) or suspected of having the fetus (or multiple fetuses). The subject may be a person that is pregnant or is suspected of being pregnant. The subject may be displaying a symptom(s) indicative of a health or physiological state or condition of the subject, such as a pregnancy-related health or physiological state or condition of the subject. As an alternative, the subject may be asymptomatic with respect to such health or physiological state or condition.

The term “pregnancy-related state,” as used herein, generally refers to any health, physiological, and/or biochemical state or condition of a subject that is pregnant or is suspected of being pregnant, or of a fetus (or multiple fetuses) of the subject. Examples of pregnancy-related states include, without limitation, pre-term birth, full-term birth, gestational age, due date, onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), placenta accreta spectrum disorders (placenta increta, placenta percreta, and placenta accreta), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and fetal development stages or states (e.g., normal fetal organ function or development, and abnormal fetal organ function or development). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus. In some situations, the pregnancy-related state is not associated with the health or physiological state or condition of a fetus (or multiple fetuses) of the subject.

As used herein, the term “sample,” generally refers to a biological sample obtained from or derived from one or more subjects. Biological samples may be cell-free biological samples or substantially cell-free biological samples, or may be processed or fractionated to produce cell-free biological samples. For example, cell-free biological samples may include cell-free ribonucleic acid (cfRNA), cell-free deoxyribonucleic acid (cfDNA), cell-free fetal DNA (cffDNA), plasma, serum, urine, saliva, amniotic fluid, and derivatives thereof. Cell-free biological samples may be obtained or derived from subjects using an ethylenediaminetetraacetic acid (EDTA) collection tube, a cell-free RNA collection tube (e.g., Streck), or a cell-free DNA collection tube (e.g., Streck). Cell-free biological samples may be derived from whole blood samples by fractionation. Biological samples or derivatives thereof may contain cells. For example, a biological sample may be a blood sample or a derivative thereof (e.g., blood collected by a collection tube or blood drops), a vaginal sample (e.g., a vaginal swab), or a cervical sample (e.g., a cervical swab).

As used herein, the term “nucleic acid” generally refers to a polymeric form of nucleotides of any length, either deoxyribonucleotides (dNTPs) or ribonucleotides (rNTPs), or analogs thereof. Nucleic acids may have any three-dimensional structure, and may perform any function, known or unknown. Non-limiting examples of nucleic acids include deoxyribonucleic (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent.

As used herein, the term “target nucleic acid” generally refers to a nucleic acid molecule in a starting population of nucleic acid molecules having a nucleotide sequence whose presence, amount, and/or sequence, or changes in one or more of these, are desired to be determined. A target nucleic acid may be any type of nucleic acid, including DNA, RNA, and analogs thereof. As used herein, a “target ribonucleic acid (RNA)” generally refers to a target nucleic acid that is RNA. As used herein, a “target deoxyribonucleic acid (DNA)” generally refers to a target nucleic acid that is DNA.

As used herein, the terms “amplifying” and “amplification” generally refer to increasing the size or quantity of a nucleic acid molecule. The nucleic acid molecule may be single-stranded or double-stranded. Amplification may include generating one or more copies or “amplified product” of the nucleic acid molecule. Amplification may be performed, for example, by extension (e.g., primer extension) or ligation. Amplification may include performing a primer extension reaction to generate a strand complementary to a single-stranded nucleic acid molecule, and in some cases generate one or more copies of the strand and/or the single-stranded nucleic acid molecule. The term “DNA amplification” generally refers to generating one or more copies of a DNA molecule or “amplified DNA product.” The term “reverse transcription amplification” generally refers to the generation of deoxyribonucleic acid (DNA) from a ribonucleic acid (RNA) template via the action of a reverse transcriptase.

Every year, about 15 million pre-term births are reported globally. Pre-term birth may affect as many as about 10% of pregnancies, of which the majority are spontaneous pre-term births. Currently, there may be no meaningful, clinically actionable diagnostic screenings or tests available for many pregnancy-related complications such as pre-term birth. However, pregnancy-related complications such as pre-term birth are a leading cause of neonatal death and of complications later in life. Further, such pregnancy-related complications may cause negative health effects on maternal health. Thus, to make pregnancy as safe as possible, there exists a need for rapid, accurate methods for identifying and monitoring pregnancy-related states that are non-invasive and cost-effective, toward improving maternal and fetal health.

Current tests for prenatal care may be in inaccessible and incomplete. For cases in which pregnancies progress without pregnancy-related complications, limited methods of pregnancy monitoring may be available for a pregnancy subject, such as molecular tests, ultrasound imaging, and estimation of gestational age and/or due date using the last menstrual period. However, such monitoring methods may be complex, expensive, and unreliable. For example, molecular tests cannot predict gestational age, ultrasound imaging is expensive and best performed during the first trimester of pregnancy, and estimation of gestational age and/or due date using the last menstrual period may be unreliable. Further, for cases in which pregnancies progress with pregnancy-related complications such as risk of spontaneous pre-term delivery, the clinical utility of molecular tests, ultrasound imaging, and demographic factors may be limited. For example, molecular tests may have a limited BMI (body mass index) range, a limited gestational age and/or due date range (about 2 weeks), and a low positive predictive value (PPV); ultrasound imaging may be expensive and have low PPV and specificity; and the use of demographic factors to predict risk of pregnancy-related complications may be unreliable. Therefore, there exists an urgent clinical need for accurate and affordable non-invasive diagnostic methods for detection and monitoring of pregnancy-related states (e.g., estimation of gestational age, due date, and/or onset of labor, and prediction of pregnancy-related complications such as pre-term birth) toward clinically actionable outcomes.

The present disclosure provides methods, systems, and kits for identifying or monitoring pregnancy-related states by processing cell-free biological samples obtained from or derived from subjects (e.g., pregnancy female subjects). Cell-free biological samples (e.g., plasma samples) obtained from subjects may be analyzed to identify the pregnancy-related state (which may include, e.g., measuring a presence, absence, or quantitative assessment (e.g., risk) of the pregnancy-related state). Such subjects may include subjects with one or more pregnancy-related states and subjects without pregnancy-related states. Pregnancy-related states may include, for example, pre-term birth, full-term birth, gestational age, due date, onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), placenta accreta spectrum disorders (placenta increta, placenta percreta, and placenta accreta), intrauterine/fetal growth restriction, and macrosomia (large fetus for gestational age). In some embodiments, pregnancy-related states are not associated with the health of a fetus. In some embodiments, pregnancy-related states include neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea) and fetal development stages or states (e.g., normal fetal organ function or development, and abnormal fetal organ function or development). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

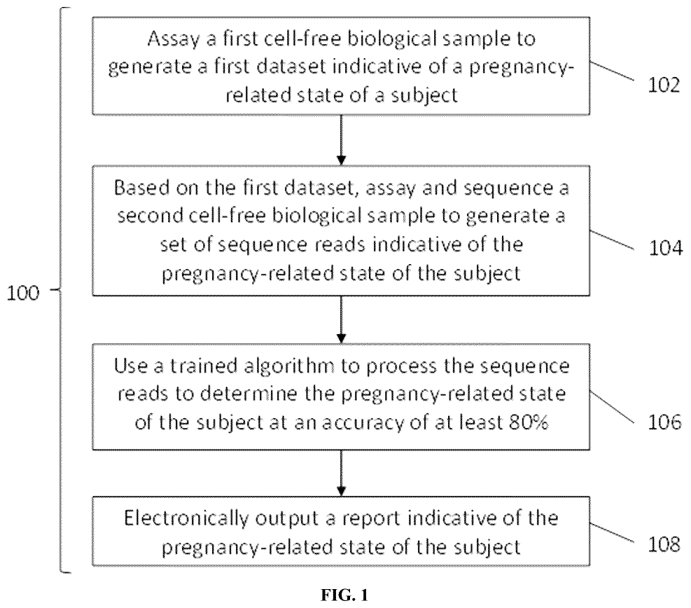

FIG. 1 illustrates an example workflow of a method for identifying or monitoring a pregnancy-related state of a subject, in accordance with disclosed embodiments. In an aspect, the present disclosure provides a method 100 for identifying or monitoring a pregnancy-related state of a subject. The method 100 may comprise using a first assay to process a first cell-free biological sample derived from the subject to generate a first dataset (as in operation 102). Next, based at least in part on the first dataset generated, the method 100 may optionally comprise using a second assay (e.g., different from the first assay) to process a second cell-free biological sample derived from the subject to generate a second dataset indicative of the pregnancy-related state at a specificity greater than the first dataset. For example, ribonucleic acid (RNA) molecules extracted from a second cell-free plasma sample may be sequenced to generate a set of sequence reads indicative of a pregnancy-related state of the subject (as in operation 104). In some embodiments, a first cell-free biological sample may be obtained from a subject at a first time point for processing with a first assay. Then, optionally a second cell-free biological sample may be obtained from the same subject at a second time point for processing with a second assay. In some embodiments, a cell-free biological sample may be obtained from a subject and then aliquoted to produce a first cell-free biological sample and a second cell-free biological sample, which are then processed with a first assay and a second assay, respectively. Next, a trained algorithm may be used to process the first dataset and/or the second dataset to determine the pregnancy-related state of the subject (as in operation 106). The trained algorithm may be configured to identify the pregnancy-related state at an accuracy of at least about 80% over 50 independent samples. A report may then be electronically outputted that is indicative of (e.g., identifies or provides an indication of) presence or susceptibility of the pregnancy-related state of the subject (as in operation 108).

Assaying Cell-Free Biological Samples

The cell-free biological samples may be obtained or derived from a human subject (e.g., a pregnant female subject). The cell-free biological samples may be stored in a variety of storage conditions before processing, such as different temperatures (e.g., at room temperature, under refrigeration or freezer conditions, at 25° C., at 4° C., at −18° C., −20° C., or at −80° C.) or different suspensions (e.g., EDTA collection tubes, cell-free RNA collection tubes, or cell-free DNA collection tubes).

The cell-free biological sample may be obtained from a subject with a pregnancy-related state (e.g., a pregnancy-related complication), from a subject that is suspected of having a pregnancy-related state (e.g., a pregnancy-related complication), or from a subject that does not have or is not suspected of having the pregnancy-related state (e.g., a pregnancy-related complication). The pregnancy-related state may comprise a pregnancy-related complication, such as pre-term birth, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), placenta accreta spectrum disorders (placenta increta, placenta percreta, and placenta accreta), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and abnormal fetal development stages or states (e.g., abnormal fetal organ function or development). The pregnancy-related state may comprise a full-term birth, normal fetal development stages or states (e.g., normal fetal organ function or development), or absence of a pregnancy-related complication (e.g., pre-term birth, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), placenta accreta spectrum disorders (placenta increta, placenta percreta, and placenta accreta), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and abnormal fetal development stages or states (e.g., abnormal fetal organ function or development)). The pregnancy-related state may comprise a quantitative assessment of pregnancy such as gestational age (e.g., measured in days, weeks or months) or due date (e.g., expressed as a predicted or estimated calendar date or range of calendar dates). The pregnancy-related state may comprise a quantitative assessment of a pregnancy-related complication such as a likelihood, a susceptibility, or a risk (e.g., expressed as a probability, a relative probability, an odds ratio, or a risk score or risk index) of the pregnancy-related complication (e.g., pre-term birth, onset of labor, pregnancy-related hypertensive disorders (e.g., preeclampsia), eclampsia, gestational diabetes, a congenital disorder of a fetus of the subject, ectopic pregnancy, spontaneous abortion, stillbirth, post-partum complications (e.g., post-partum depression, hemorrhage or excessive bleeding, pulmonary embolism, cardiomyopathy, diabetes, anemia, and hypertensive disorders), hyperemesis gravidarum (morning sickness), hemorrhage or excessive bleeding during delivery, premature rupture of membrane, premature rupture of membrane in pre-term birth, placenta previa (placenta covering the cervix), placenta accreta spectrum disorders (placenta increta, placenta percreta, and placenta accreta), intrauterine/fetal growth restriction, macrosomia (large fetus for gestational age), neonatal conditions (e.g., anemia, apnea, bradycardia and other heart defects, bronchopulmonary dysplasia or chronic lung disease, diabetes, gastroschisis, hydrocephaly, hyperbilirubinemia, hypocalcemia, hypoglycemia, intraventricular hemorrhage, jaundice, necrotizing enterocolitis, patent ductus arteriosis, periventricular leukomalacia, persistent pulmonary hypertension, polycythemia, respiratory distress syndrome, retinopathy of prematurity, and transient tachypnea), and abnormal fetal development stages or states (e.g., abnormal fetal organ function or development)). For example, the pregnancy-related state may comprise a likelihood or susceptibility of an onset of labor in the future (e.g., within about 1 hour, about 2 hours, about 4 hours, about 6 hours, about 8 hours, about 10 hours, about 12 hours, about 14 hours, about 16 hours, about 18 hours, about 20 hours, about 22 hours, about 24 hours, about 1.5 days, about 2 days, about 2.5 days, about 3 days, about 3.5 days, about 4 days, about 4.5 days, about 5 days, about 5.5 days, about 6 days, about 6.5 days, about 7 days, about 8 days, about 9 days, about 10 days, about 12 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 13 weeks, or more than about 13 weeks). For example, the fetal development stages or states may be related to normal fetal organ function or development and/or abnormal fetal organ function or development for a fetal organ selected from the group consisting of heart, large intestine, small intestine, retina, prefrontal cortex, midbrain, kidney, and esophagus.

The cell-free biological sample may be taken before and/or after treatment of a subject with the pregnancy-related complication. Cell-free biological samples may be obtained from a subject during a treatment or a treatment regime. Multiple cell-free biological samples may be obtained from a subject to monitor the effects of the treatment over time. The cell-free biological sample may be taken from a subject known or suspected of having a pregnancy-related state (e.g., pregnancy-related complication) for which a definitive positive or negative diagnosis is not available via clinical tests. The sample may be taken from a subject suspected of having a pregnancy-related complication. The cell-free biological sample may be taken from a subject experiencing unexplained symptoms, such as fatigue, nausea, weight loss, aches and pains, weakness, or bleeding. The cell-free biological sample may be taken from a subject having explained symptoms. The cell-free biological sample may be taken from a subject at risk of developing a pregnancy-related complication due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

The cell-free biological sample may contain one or more analytes capable of being assayed, such as cell-free ribonucleic acid (cfRNA) molecules suitable for assaying to generate transcriptomic data, cell-free deoxyribonucleic acid (cfDNA) molecules suitable for assaying to generate genomic data, proteins suitable for assaying to generate proteomic data, metabolites suitable for assaying to generate metabolomic data, or a mixture or combination thereof. One or more such analytes (e.g., cfRNA molecules, cfDNA molecules, proteins, or metabolites) may be isolated or extracted from one or more cell-free biological samples of a subject for downstream assaying using one or more suitable assays.

After obtaining a cell-free biological sample from the subject, the cell-free biological sample may be processed to generate datasets indicative of a pregnancy-related state of the subject. For example, a presence, absence, or quantitative assessment of nucleic acid molecules of the cell-free biological sample at a panel of pregnancy-related state-associated loci (e.g., quantitative measures of RNA transcripts or DNA at the pregnancy-related state-associated loci), proteomic data comprising quantitative measures of proteins of the dataset at a panel of pregnancy-related state-associated proteins, and/or metabolome data comprising quantitative measures of a panel of pregnancy-related state-associated metabolites may be indicative of a pregnancy-related state. Processing the cell-free biological sample obtained from the subject may comprise (i) subjecting the cell-free biological sample to conditions that are sufficient to isolate, enrich, or extract a plurality of nucleic acid molecules, proteins, and/or metabolites, and (ii) assaying the plurality of nucleic acid molecules, proteins, and/or metabolites to generate the dataset.

In some embodiments, a plurality of nucleic acid molecules is extracted from the cell-free biological sample and subjected to sequencing to generate a plurality of sequencing reads. The nucleic acid molecules may comprise ribonucleic acid (RNA) or deoxyribonucleic acid (DNA). The nucleic acid molecules (e.g., RNA or DNA) may be extracted from the cell-free biological sample by a variety of methods, such as a FastDNA Kit protocol from MP Biomedicals, a QIAamp DNA cell-free biological mini kit from Qiagen, or a cell-free biological DNA isolation kit protocol from Norgen Biotek. The extraction method may extract all RNA or DNA molecules from a sample. Alternatively, the extract method may selectively extract a portion of RNA or DNA molecules from a sample. Extracted RNA molecules from a sample may be converted to DNA molecules by reverse transcription (RT).

In some embodiments, the method comprises the assaying the hydroxymethylated cell-free DNA in the cell-free biological sample derived from the subject to detect the set of biomarkers. In some embodiments, the changes of DNA methylation states are assayed by enriching 5hmC containing DNA with subsequent nucleic acid sequencing. In some embodiments, the 5hmC containing DNA fragments are enriched by affinity based methods.

The sequencing may be performed by any suitable sequencing methods, such as massively parallel sequencing (MPS), paired-end sequencing, high-throughput sequencing, next-generation sequencing (NGS), shotgun sequencing, single-molecule sequencing, nanopore sequencing, semiconductor sequencing, pyrosequencing, sequencing-by-synthesis (SBS), sequencing-by-ligation, sequencing-by-hybridization, and RNA-Seq (Illumina).

The sequencing may comprise nucleic acid amplification (e.g., of RNA or DNA molecules). In some embodiments, the nucleic acid amplification is polymerase chain reaction (PCR). A suitable number of rounds of PCR (e.g., PCR, qPCR, reverse-transcriptase PCR, digital PCR, etc.) may be performed to sufficiently amplify an initial amount of nucleic acid (e.g., RNA or DNA) to a desired input quantity for subsequent sequencing. In some cases, the PCR may be used for global amplification of target nucleic acids. This may comprise using adapter sequences that may be first ligated to different molecules followed by PCR amplification using universal primers. PCR may be performed using any of a number of commercial kits, e.g., provided by Life Technologies, Affymetrix, Promega, Qiagen, etc. In other cases, only certain target nucleic acids within a population of nucleic acids may be amplified. Specific primers, possibly in conjunction with adapter ligation, may be used to selectively amplify certain targets for downstream sequencing. The PCR may comprise targeted amplification of one or more loci, such as loci associated with pregnancy-related states. The sequencing may comprise use of simultaneous reverse transcription (RT) and polymerase chain reaction (PCR), such as a OneStep RT-PCR kit protocol by Qiagen, NEB, Thermo Fisher Scientific, or Bio-Rad.

RNA or DNA molecules isolated or extracted from a cell-free biological sample may be tagged, e.g., with identifiable tags, to allow for multiplexing of a plurality of samples. Any number of RNA or DNA samples may be multiplexed. For example a multiplexed reaction may contain RNA or DNA from at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 initial cell-free biological samples. For example, a plurality of cell-free biological samples may be tagged with sample barcodes such that each DNA molecule may be traced back to the sample (and the subject) from which the DNA molecule originated. Such tags may be attached to RNA or DNA molecules by ligation or by PCR amplification with primers.

After subjecting the nucleic acid molecules to sequencing, suitable bioinformatics processes may be performed on the sequence reads to generate the data indicative of the presence, absence, or relative assessment of the pregnancy-related state. For example, the sequence reads may be aligned to one or more reference genomes (e.g., a genome of one or more species such as a human genome). The aligned sequence reads may be quantified at one or more loci to generate the datasets indicative of the pregnancy-related state. For example, quantification of sequences corresponding to a plurality of loci associated with pregnancy-related states may generate the datasets indicative of the pregnancy-related state.

The cell-free biological sample may be processed without any nucleic acid extraction. For example, the pregnancy-related state may be identified or monitored in the subject by using probes configured to selectively enrich nucleic acid (e.g., RNA or DNA) molecules corresponding to the plurality of pregnancy-related state-associated loci. The probes may be nucleic acid primers. The probes may have sequence complementarity with nucleic acid sequences from one or more of the plurality of pregnancy-related state-associated loci or genomic regions. The plurality of pregnancy-related state-associated loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more distinct pregnancy-related state-associated loci or genomic regions. The plurality of pregnancy-related state-associated loci or genomic regions may comprise one or more members (e.g., 1, 2, 3, 4, 5, 6, 7, 8, 9, 10, 11, 12, 13, 14, 15, 16, 17, 18, 19, 20, about 25, about 30, about 35, about 40, about 45, about 50, about 55, about 60, about 65, about 70, about 75, about 80, or more) selected from the group consisting genes listed in Table 3, non-genic loci listed in Table 4, genes listed in Table 5, non-genic loci listed in Table 6, genes listed in Table 7, and genes listed in Table 9. In some embodiments, the panel of the one or more loci comprises a locus associated with preeclampsia, wherein the locus is selected from the group consisting of group consisting of genomic and non-genomic or aggregated loci listed in Table 11, Table 12, Table 13, and the CLDN7, PAPPA2, SNORD14A, PLEKHH1, MAGEA10, TLE6, and FABP11 genes.

The probes may be nucleic acid molecules (e.g., RNA or DNA) having sequence complementarity with nucleic acid sequences (e.g., RNA or DNA) of the one or more loci (e.g., pregnancy-related state-associated loci). These nucleic acid molecules may be primers or enrichment sequences. The assaying of the cell-free biological sample using probes that are selective for the one or more loci (e.g., pregnancy-related state-associated loci) may comprise use of array hybridization (e.g., microarray-based), polymerase chain reaction (PCR), or nucleic acid sequencing (e.g., RNA sequencing or DNA sequencing). In some embodiments, DNA or RNA may be assayed by one or more of: isothermal DNA/RNA amplification methods (e.g., loop-mediated isothermal amplification (LAMP), helicase dependent amplification (HDA), rolling circle amplification (RCA), recombinase polymerase amplification (RPA)), immunoassays, electrochemical assays, surface-enhanced Raman spectroscopy (SERS), quantum dot (QD)-based assays, molecular inversion probes, droplet digital PCR (ddPCR), CRISPR/Cas-based detection (e.g., CRISPR-typing PCR (ctPCR), specific high-sensitivity enzymatic reporter un-locking (SHERLOCK), DNA endonuclease targeted CRISPR trans reporter (DETECTR), and CRISPR-mediated analog multi-event recording apparatus (CAMERA)), and laser transmission spectroscopy (LTS).

The assay readouts may be quantified at one or more loci (e.g., pregnancy-related state-associated loci) to generate the data indicative of the pregnancy-related state. For example, quantification of array hybridization or polymerase chain reaction (PCR) corresponding to a plurality of loci (e.g., pregnancy-related state-associated loci) may generate data indicative of the pregnancy-related state. Assay readouts may comprise quantitative PCR (qPCR) values, digital PCR (dPCR) values, digital droplet PCR (ddPCR) values, fluorescence values, etc., or normalized values thereof. The assay may be a home use test configured to be performed in a home setting.

In some embodiments, multiple assays are used to process cell-free biological samples of a subject. For example, a first assay may be used to process a first cell-free biological sample obtained or derived from the subject to generate a first dataset; and based at least in part on the first dataset, a second assay different from the first assay may be used to process a second cell-free biological sample obtained or derived from the subject to generate a second dataset indicative of the pregnancy-related state. The first assay may be used to screen or process cell-free biological samples of a set of subjects, while the second or subsequent assays may be used to screen or process cell-free biological samples of a smaller subset of the set of subjects. The first assay may have a low cost and/or a high sensitivity of detecting one or more pregnancy-related states (e.g., pregnancy-related complication), that is amenable to screening or processing cell-free biological samples of a relatively large set of subjects. The second assay may have a higher cost and/or a higher specificity of detecting one or more pregnancy-related states (e.g., pregnancy-related complication), that is amenable to screening or processing cell-free biological samples of a relatively small set of subjects (e.g., a subset of the subjects screened using the first assay). The second assay may generate a second dataset having a specificity (e.g., for one or more pregnancy-related states such as pregnancy-related complications) greater than the first dataset generated using the first assay. As an example, one or more cell-free biological samples may be processed using a cfRNA assay on a large set of subjects and subsequently a metabolomics assay on a smaller subset of subjects, or vice versa. The smaller subset of subjects may be selected based at least in part on the results of the first assay.

Alternatively, multiple assays may be used to simultaneously process cell-free biological samples of a subject. For example, a first assay may be used to process a first cell-free biological sample obtained or derived from the subject to generate a first dataset indicative of the pregnancy-related state; and a second assay different from the first assay may be used to process a second cell-free biological sample obtained or derived from the subject to generate a second dataset indicative of the pregnancy-related state. Any or all of the first dataset and the second dataset may then be analyzed to assess the pregnancy-related state of the subject. For example, a single diagnostic index or diagnosis score may be generated based on a combination of the first dataset and the second dataset. As another example, separate diagnostic indexes or diagnosis scores may be generated based on the first dataset and the second dataset.

The cell-free biological samples may be processed to identify a set of biomarker RNA transcripts that are indicative of a set of corresponding biomarker proteins, pathways, and/or metabolites. For example, a given biomarker RNA transcript may be expected to be translated into a corresponding given biomarker protein or a gene regulator for a corresponding given biomarker protein. Therefore, identifying a presence or absence of the given biomarker RNA transcript in a biological sample may be indicative of a presence or absence of a corresponding biomarker protein. As another example, a given biomarker RNA transcript may be expected to correlate with a corresponding given pathway. Therefore, identifying a presence or absence of the given biomarker RNA transcript in a biological sample may be indicative of a presence or absence of the corresponding pathway activity. As another example, a given biomarker RNA transcript may be expected to correlate with a corresponding given biomarker metabolite. Therefore, identifying a presence or absence of the given biomarker RNA transcript in a biological sample may be indicative of a presence or absence of the corresponding biomarker metabolite. In some embodiments, the set of corresponding biomarker proteins, pathways, and/or metabolites comprises pregnancy-related state-associated proteins, pathways, and/or metabolites. In some embodiments, the set of corresponding biomarker proteins, pathways, and/or metabolites comprises placental proteins, pathways, and/or metabolites. For example, identifying a presence or absence of the PAPPA gene may be indicative of a presence or absence of the PAPPA protein analog.

The cell-free biological samples may be processed using a methylation-specific assay. For example, a methylation-specific assay may be used to identify a quantitative measure (e.g., indicative of a presence, absence, or relative amount) of methylation each of a plurality of pregnancy-related state-associated loci in a cell-free biological sample of the subject. The methylation-specific assay may be configured to process cell-free biological samples such as a blood sample or a urine sample (or derivatives thereof) of the subject. A quantitative measure (e.g., indicative of a presence, absence, or relative amount) of methylation of pregnancy-related state-associated loci in the cell-free biological sample may be indicative of one or more pregnancy-related states. The methylation-specific assay may be used to generate datasets indicative of the quantitative measure (e.g., indicative of a presence, absence, or relative amount) of methylation of each of a plurality of pregnancy-related state-associated loci in the cell-free biological sample of the subject.

The methylation-specific assay may comprise, for example, one or more of: a methylation-aware sequencing (e.g., using bisulfite treatment), pyrosequencing, methylation-sensitive single-strand conformation analysis (MS-SSCA), high-resolution melting analysis (HIRM), methylation-sensitive single-nucleotide primer extension (MS-SnuPE), base-specific cleavage/MALDI-TOF, microarray-based methylation assay, methylation-specific PCR, targeted bisulfite sequencing, oxidative bisulfite sequencing, mass spectroscopy-based bisulfite sequencing, or reduced representation bisulfite sequence (RRBS).

The methylation-specific assay may for example assaying the hydroxymethylated cell-free DNA in the cell-free biological sample. The changes of methylation DNA may be detected by enriching 5hmC containing DNA fragments.

Kits

The present disclosure provides kits for identifying or monitoring a pregnancy-related state of a subject. A kit may comprise probes for identifying a quantitative measure (e.g., indicative of a presence, absence, or relative amount) of sequences at each of a plurality of pregnancy-related state-associated loci in a cell-free biological sample of the subject. A quantitative measure (e.g., indicative of a presence, absence, or relative amount) of sequences at each of a plurality of pregnancy-related state-associated loci in the cell-free biological sample may be indicative of one or more pregnancy-related states. The probes may be selective for the sequences at the plurality of pregnancy-related state-associated loci in the cell-free biological sample. A kit may comprise instructions for using the probes to process the cell-free biological sample to generate datasets indicative of a quantitative measure (e.g., indicative of a presence, absence, or relative amount) of sequences at each of the plurality of pregnancy-related state-associated loci in a cell-free biological sample of the subject.

The probes in the kit may be selective for the sequences at the plurality of pregnancy-related state-associated loci in the cell-free biological sample. The probes in the kit may be configured to selectively enrich nucleic acid (e.g., RNA or DNA) molecules corresponding to the plurality of pregnancy-related state-associated loci. The probes in the kit may be nucleic acid primers. The probes in the kit may have sequence complementarity with nucleic acid sequences from one or more of the plurality of pregnancy-related state-associated loci or genomic regions. The plurality of pregnancy-related state-associated loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more distinct pregnancy-related state-associated loci or genomic regions. The plurality of pregnancy-related state-associated loci or genomic regions may comprise one or more members selected from the group consisting of genes listed in Table 3, non-genic loci listed in Table 4, genes listed in Table 5, non-genic loci listed in Table 6, genes listed in Table 7, genes listed in Table 9, and group consisting of genomic and non-genomic or aggregated loci listed in Table 11, Table 12, Table 13, and the CLDN7, PAPPA2, SNORD14A, PLEKHH1, MAGEA10, TLE6, and FABP1l genes.

The instructions in the kit may comprise instructions to assay the cell-free biological sample using the probes that are selective for the sequences at the plurality of pregnancy-related state-associated loci in the cell-free biological sample. These probes may be nucleic acid molecules (e.g., RNA or DNA) having sequence complementarity with nucleic acid sequences (e.g., RNA or DNA) from one or more of the plurality of pregnancy-related state-associated loci. These nucleic acid molecules may be primers or enrichment sequences. The instructions to assay the cell-free biological sample may comprise introductions to perform array hybridization, polymerase chain reaction (PCR), or nucleic acid sequencing (e.g., DNA sequencing or RNA sequencing) to process the cell-free biological sample to generate datasets indicative of a quantitative measure (e.g., indicative of a presence, absence, or relative amount) of sequences at each of the plurality of pregnancy-related state-associated loci in the cell-free biological sample. A quantitative measure (e.g., indicative of a presence, absence, or relative amount) of sequences at each of a plurality of pregnancy-related state-associated loci in the cell-free biological sample may be indicative of one or more pregnancy-related states.

The instructions in the kit may comprise instructions to measure and interpret assay readouts, which may be quantified at one or more of the plurality of pregnancy-related state-associated loci to generate the datasets indicative of a quantitative measure (e.g., indicative of a presence, absence, or relative amount) of sequences at each of the plurality of pregnancy-related state-associated loci in the cell-free biological sample. For example, quantification of array hybridization or polymerase chain reaction (PCR) corresponding to the plurality of pregnancy-related state-associated loci may generate the datasets indicative of a quantitative measure (e.g., indicative of a presence, absence, or relative amount) of sequences at each of the plurality of pregnancy-related state-associated loci in the cell-free biological sample. Assay readouts may comprise quantitative PCR (qPCR) values, digital PCR (dPCR) values, digital droplet PCR (ddPCR) values, fluorescence values, etc., or normalized values thereof.

Trained Algorithms

After using one or more assays to process one or more cell-free biological samples derived from the subject to generate one or more datasets indicative of the pregnancy-related state or pregnancy-related complication, a trained algorithm may be used to process one or more of the datasets (e.g., at each of a plurality of pregnancy-related state-associated loci) to determine the pregnancy-related state. For example, the trained algorithm may be used to determine quantitative measures of sequences at each of the plurality of pregnancy-related state-associated loci in the cell-free biological samples. The trained algorithm may be configured to identify the pregnancy-related state with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99% for at least about 25, at least about 50, at least about 100, at least about 150, at least about 200, at least about 250, at least about 300, at least about 350, at least about 400, at least about 450, at least about 500, or more than about 500 independent samples.

The trained algorithm may comprise a supervised machine learning algorithm. The trained algorithm may comprise a classification and regression tree (CART) algorithm. The supervised machine learning algorithm may comprise, for example, a Random Forest, a support vector machine (SVM), a neural network, or a deep learning algorithm. The trained algorithm may comprise a differential expression algorithm. The differential expression algorithm may comprise a use comparison of stochastic models, generalized Poisson (GPseq), mixed Poisson (TSPM), Poisson log-linear (PoissonSeq), negative binomial (edgeR, DESeq, baySeq, NBPSeq), linear model fit by MAANOVA, or a combination thereof. The trained algorithm may comprise an unsupervised machine learning algorithm.

The trained algorithm may be configured to accept a plurality of input variables and to produce one or more output values based on the plurality of input variables. The plurality of input variables may comprise one or more datasets indicative of a pregnancy-related state. For example, an input variable may comprise a number of sequences corresponding to or aligning to each of the plurality of pregnancy-related state-associated loci. The plurality of input variables may also include clinical health data of a subject.

The trained algorithm may comprise a classifier, such that each of the one or more output values comprises one of a fixed number of possible values (e.g., a linear classifier, a logistic regression classifier, etc.) indicating a classification of the cell-free biological sample by the classifier. The trained algorithm may comprise a binary classifier, such that each of the one or more output values comprises one of two values (e.g., {0, 1}, {positive, negative}, or {high-risk, low-risk}) indicating a classification of the cell-free biological sample by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the cell-free biological sample by the classifier. The output values may comprise descriptive labels, numerical values, or a combination thereof. Some of the output values may comprise descriptive labels. Such descriptive labels may provide an identification or indication of the disease or disorder state of the subject, and may comprise, for example, positive, negative, high-risk, intermediate-risk, low-risk, or indeterminate. Such descriptive labels may provide an identification of a treatment for the subject's pregnancy-related state, and may comprise, for example, a therapeutic intervention, a duration of the therapeutic intervention, and/or a dosage of the therapeutic intervention suitable to treat a pregnancy-related condition. Such descriptive labels may provide an identification of secondary clinical tests that may be appropriate to perform on the subject, and may comprise, for example, an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof. For example, such descriptive labels may provide a prognosis of the pregnancy-related state of the subject. As another example, such descriptive labels may provide a relative assessment of the pregnancy-related state (e.g., an estimated gestational age in number of days, weeks, or months) of the subject. Some descriptive labels may be mapped to numerical values, for example, by mapping “positive” to 1 and “negative” to 0.

Some of the output values may comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the pregnancy-related state of the subject. Some numerical values may be mapped to descriptive labels, for example, by mapping 1 to “positive” and 0 to “negative.”

Some of the output values may be assigned based on one or more cutoff values. For example, a binary classification of samples may assign an output value of “positive” or 1 if the sample indicates that the subject has at least a 50% probability of having a pregnancy-related state (e.g., pregnancy-related complication). For example, a binary classification of samples may assign an output value of “negative” or 0 if the sample indicates that the subject has less than a 50% probability of having a pregnancy-related state (e.g., pregnancy-related complication). In this case, a single cutoff value of 50% is used to classify samples into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, a classification of samples may assign an output value of “positive” or 1 if the sample indicates that the subject has a probability of having a pregnancy-related state (e.g., pregnancy-related complication) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of having a pregnancy-related state (e.g., pregnancy-related complication) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classification of samples may assign an output value of “negative” or 0 if the sample indicates that the subject has a probability of having a pregnancy-related state (e.g., pregnancy-related complication) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having a pregnancy-related state (e.g., pregnancy-related complication) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classification of samples may assign an output value of “indeterminate” or 2 if the sample is not classified as “positive”, “negative”, 1, or 0. In this case, a set of two cutoff values is used to classify samples into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values, where n is any positive integer.

The trained algorithm may be trained with a plurality of independent training samples. Each of the independent training samples may comprise a cell-free biological sample from a subject, associated datasets obtained by assaying the cell-free biological sample (as described elsewhere herein), and one or more known output values corresponding to the cell-free biological sample (e.g., a clinical diagnosis, prognosis, absence, or treatment efficacy of a pregnancy-related state of the subject). Independent training samples may comprise cell-free biological samples and associated datasets and outputs obtained or derived from a plurality of different subjects. Independent training samples may comprise cell-free biological samples and associated datasets and outputs obtained at a plurality of different time points from the same subject (e.g., on a regular basis such as weekly, biweekly, or monthly). Independent training samples may be associated with presence of the pregnancy-related state (e.g., training samples comprising cell-free biological samples and associated datasets and outputs obtained or derived from a plurality of subjects known to have the pregnancy-related state). Independent training samples may be associated with absence of the pregnancy-related state (e.g., training samples comprising cell-free biological samples and associated datasets and outputs obtained or derived from a plurality of subjects who are known to not have a previous diagnosis of the pregnancy-related state or who have received a negative test result for the pregnancy-related state).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise cell-free biological samples associated with presence of the pregnancy-related state and/or cell-free biological samples associated with absence of the pregnancy-related state. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the pregnancy-related state. In some embodiments, the cell-free biological sample is independent of samples used to train the trained algorithm.

The trained algorithm may be trained with a first number of independent training samples associated with presence of the pregnancy-related state and a second number of independent training samples associated with absence of the pregnancy-related state. The first number of independent training samples associated with presence of the pregnancy-related state may be no more than the second number of independent training samples associated with absence of the pregnancy-related state. The first number of independent training samples associated with presence of the pregnancy-related state may be equal to the second number of independent training samples associated with absence of the pregnancy-related state. The first number of independent training samples associated with presence of the pregnancy-related state may be greater than the second number of independent training samples associated with absence of the pregnancy-related state.

The trained algorithm may be configured to identify the pregnancy-related state at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the pregnancy-related state by the trained algorithm may be calculated as the percentage of independent test samples (e.g., subjects known to have the pregnancy-related state or subjects with negative clinical test results for the pregnancy-related state) that are correctly identified or classified as having or not having the pregnancy-related state.

The trained algorithm may be configured to identify the pregnancy-related state with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the pregnancy-related state using the trained algorithm may be calculated as the percentage of cell-free biological samples identified or classified as having the pregnancy-related state that correspond to subjects that truly have the pregnancy-related state.

The trained algorithm may be configured to identify the pregnancy-related state with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the pregnancy-related state using the trained algorithm may be calculated as the percentage of cell-free biological samples identified or classified as not having the pregnancy-related state that correspond to subjects that truly do not have the pregnancy-related state.

The trained algorithm may be configured to identify the pregnancy-related state with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the pregnancy-related state using the trained algorithm may be calculated as the percentage of independent test samples associated with presence of the pregnancy-related state (e.g., subjects known to have the pregnancy-related state) that are correctly identified or classified as having the pregnancy-related state.

The trained algorithm may be configured to identify the pregnancy-related state with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the pregnancy-related state using the trained algorithm may be calculated as the percentage of independent test samples associated with absence of the pregnancy-related state (e.g., subjects with negative clinical test results for the pregnancy-related state) that are correctly identified or classified as not having the pregnancy-related state.

The trained algorithm may be configured to identify the pregnancy-related state with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying cell-free biological samples as having or not having the pregnancy-related state.

The trained algorithm may be adjusted or tuned to improve one or more of the performance, accuracy, PPV, NPV, clinical sensitivity, clinical specificity, or AUC of identifying the pregnancy-related state. The trained algorithm may be adjusted or tuned by adjusting parameters of the trained algorithm (e.g., a set of cutoff values used to classify a cell-free biological sample as described elsewhere herein, or weights of a neural network). The trained algorithm may be adjusted or tuned continuously during the training process or after the training process has completed.

After the trained algorithm is initially trained, a subset of the inputs may be identified as most influential or most important to be included for making high-quality classifications. For example, a subset of the plurality of pregnancy-related state-associated loci may be identified as most influential or most important to be included for making high-quality classifications or identifications of pregnancy-related states (or sub-types of pregnancy-related states). The plurality of pregnancy-related state-associated loci or a subset thereof may be ranked based on classification metrics indicative of each locus's influence or importance toward making high-quality classifications or identifications of pregnancy-related states (or sub-types of pregnancy-related states). Such metrics may be used to reduce, in some cases significantly, the number of input variables (e.g., predictor variables) that may be used to train the trained algorithm to a desired performance level (e.g., based on a desired minimum accuracy, PPV, NPV, clinical sensitivity, clinical specificity, AUC, or a combination thereof). For example, if training the trained algorithm with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality may yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics.

Identifying or Monitoring a Pregnancy-Related State

After using a trained algorithm to process the dataset, the pregnancy-related state or pregnancy-related complication may be identified or monitored in the subject. The identification may be based at least in part on quantitative measures of sequence reads of the dataset at a panel of pregnancy-related state-associated loci (e.g., quantitative measures of RNA transcripts or DNA at the pregnancy-related state-associated loci), proteomic data comprising quantitative measures of proteins of the dataset at a panel of pregnancy-related state-associated proteins, and/or metabolome data comprising quantitative measures of a panel of pregnancy-related state-associated metabolites.

The pregnancy-related state may be identified in the subject at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The accuracy of identifying the pregnancy-related state by the trained algorithm may be calculated as the percentage of independent test samples (e.g., subjects known to have the pregnancy-related state or subjects with negative clinical test results for the pregnancy-related state) that are correctly identified or classified as having or not having the pregnancy-related state.

The pregnancy-related state may be identified in the subject with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the pregnancy-related state using the trained algorithm may be calculated as the percentage of cell-free biological samples identified or classified as having the pregnancy-related state that correspond to subjects that truly have the pregnancy-related state.

The pregnancy-related state may be identified in the subject with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the pregnancy-related state using the trained algorithm may be calculated as the percentage of cell-free biological samples identified or classified as not having the pregnancy-related state that correspond to subjects that truly do not have the pregnancy-related state.

The pregnancy-related state may be identified in the subject with a clinical sensitivity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the pregnancy-related state using the trained algorithm may be calculated as the percentage of independent test samples associated with presence of the pregnancy-related state (e.g., subjects known to have the pregnancy-related state) that are correctly identified or classified as having the pregnancy-related state.

The pregnancy-related state may be identified in the subject with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the pregnancy-related state using the trained algorithm may be calculated as the percentage of independent test samples associated with absence of the pregnancy-related state (e.g., subjects with negative clinical test results for the pregnancy-related state) that are correctly identified or classified as not having the pregnancy-related state.

In an aspect, the present disclosure provides a method for determining that a subject is at risk of pre-term birth, comprising assaying a cell-free biological sample derived from the subject to generate a dataset that is indicative of the pre-term birth risk at a specificity of at least 80%, and using a trained algorithm that is trained on samples independent of the cell-free biological sample to determine that the subject is at risk of pre-term birth at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more.

After the pregnancy-related state is identified in a subject, a sub-type of the pregnancy-related state (e.g., selected from among a plurality of sub-types of the pregnancy-related state) may further be identified. The sub-type of the pregnancy-related state may be determined based at least in part on the quantitative measures of sequence reads of the dataset at a panel of pregnancy-related state-associated loci (e.g., quantitative measures of RNA transcripts or DNA at the pregnancy-related state-associated loci), proteomic data comprising quantitative measures of proteins of the dataset at a panel of pregnancy-related state-associated proteins, and/or metabolome data comprising quantitative measures of a panel of pregnancy-related state-associated metabolites. For example, the subject may be identified as being at risk of a sub-type of pre-term birth (e.g., selected from among a plurality of sub-types of pre-term birth). After identifying the subject as being at risk of a sub-type of pre-term birth, a clinical intervention for the subject may be selected based at least in part on the sub-type of pre-term birth for which the subject is identified as being at risk. In some embodiments, the clinical intervention is selected from a plurality of clinical interventions (e.g., clinically indicated for different sub-types of pre-term birth).

In some embodiments, the trained algorithm may determine that the subject is at risk of pre-term birth of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more.

The trained algorithm may determine that the subject is at risk of pre-term birth at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more.

Upon identifying the subject as having the pregnancy-related state, the subject may be optionally provided with a therapeutic intervention (e.g., prescribing an appropriate course of treatment to treat the pregnancy-related state of the subject). The therapeutic intervention may comprise a prescription of an effective dose of a drug, a further testing or evaluation of the pregnancy-related state, a further monitoring of the pregnancy-related state, an induction or inhibition of labor, or a combination thereof. If the subject is currently being treated for the pregnancy-related state with a course of treatment, the therapeutic intervention may comprise a subsequent different course of treatment (e.g., to increase treatment efficacy due to non-efficacy of the current course of treatment).

The therapeutic intervention may comprise recommending the subject for a secondary clinical test to confirm a diagnosis of the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

The quantitative measures of sequence reads of the dataset at the panel of pregnancy-related state-associated loci (e.g., quantitative measures of RNA transcripts or DNA at the pregnancy-related state-associated loci), proteomic data comprising quantitative measures of proteins of the dataset at a panel of pregnancy-related state-associated proteins, and/or metabolome data comprising quantitative measures of a panel of pregnancy-related state-associated metabolites may be assessed over a duration of time to monitor a patient (e.g., subject who has pregnancy-related state or who is being treated for pregnancy-related state). In such cases, the quantitative measures of the dataset of the patient may change during the course of treatment. For example, the quantitative measures of the dataset of a patient with decreasing risk of the pregnancy-related state due to an effective treatment may shift toward the profile or distribution of a healthy subject (e.g., a subject without a pregnancy-related complication). Conversely, for example, the quantitative measures of the dataset of a patient with increasing risk of the pregnancy-related state due to an ineffective treatment may shift toward the profile or distribution of a subject with higher risk of the pregnancy-related state or a more advanced pregnancy-related state.

The pregnancy-related state of the subject may be monitored by monitoring a course of treatment for treating the pregnancy-related state of the subject. The monitoring may comprise assessing the pregnancy-related state of the subject at two or more time points. The assessing may be based at least on the quantitative measures of sequence reads of the dataset at a panel of pregnancy-related state-associated loci (e.g., quantitative measures of RNA transcripts or DNA at the pregnancy-related state-associated loci), proteomic data comprising quantitative measures of proteins of the dataset at a panel of pregnancy-related state-associated proteins, and/or metabolome data comprising quantitative measures of a panel of pregnancy-related state-associated metabolites determined at each of the two or more time points.

In some embodiments, a difference in the quantitative measures of sequence reads of the dataset at a panel of pregnancy-related state-associated loci (e.g., quantitative measures of RNA transcripts or DNA at the pregnancy-related state-associated loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of one or more clinical indications, such as (i) a diagnosis of the pregnancy-related state of the subject, (ii) a prognosis of the pregnancy-related state of the subject, (iii) an increased risk of the pregnancy-related state of the subject, (iv) a decreased risk of the pregnancy-related state of the subject, (v) an efficacy of the course of treatment for treating the pregnancy-related state of the subject, and (vi) a non-efficacy of the course of treatment for treating the pregnancy-related state of the subject.

In some embodiments, a difference in the quantitative measures of sequence reads of the dataset at a panel of pregnancy-related state-associated loci (e.g., quantitative measures of RNA transcripts or DNA at the pregnancy-related state-associated loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of a diagnosis of the pregnancy-related state of the subject. For example, if the pregnancy-related state was not detected in the subject at an earlier time point but was detected in the subject at a later time point, then the difference is indicative of a diagnosis of the pregnancy-related state of the subject. A clinical action or decision may be made based on this indication of diagnosis of the pregnancy-related state of the subject, such as, for example, prescribing a new therapeutic intervention for the subject. The clinical action or decision may comprise recommending the subject for a secondary clinical test to confirm the diagnosis of the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

In some embodiments, a difference in the quantitative measures of sequence reads of the dataset at a panel of pregnancy-related state-associated loci (e.g., quantitative measures of RNA transcripts or DNA at the pregnancy-related state-associated loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of a prognosis of the pregnancy-related state of the subject.

In some embodiments, a difference in the quantitative measures of sequence reads of the dataset at a panel of pregnancy-related state-associated loci (e.g., quantitative measures of RNA transcripts or DNA at the pregnancy-related state-associated loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of the subject having an increased risk of the pregnancy-related state. For example, if the pregnancy-related state was detected in the subject both at an earlier time point and at a later time point, and if the difference is a negative difference (e.g., the quantitative measures of sequence reads of the dataset at a panel of pregnancy-related state-associated loci (e.g., quantitative measures of RNA transcripts or DNA at the pregnancy-related state-associated loci), proteomic data comprising quantitative measures of proteins of the dataset at a panel of pregnancy-related state-associated proteins, and/or metabolome data comprising quantitative measures of a panel of pregnancy-related state-associated metabolites increased from the earlier time point to the later time point), then the difference may be indicative of the subject having an increased risk of the pregnancy-related state. A clinical action or decision may be made based on this indication of the increased risk of the pregnancy-related state, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

In some embodiments, a difference in the quantitative measures of sequence reads of the dataset at a panel of pregnancy-related state-associated loci (e.g., quantitative measures of RNA transcripts or DNA at the pregnancy-related state-associated loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of the subject having a decreased risk of the pregnancy-related state. For example, if the pregnancy-related state was detected in the subject both at an earlier time point and at a later time point, and if the difference is a positive difference (e.g., the quantitative measures of sequence reads of the dataset at a panel of pregnancy-related state-associated loci (e.g., quantitative measures of RNA transcripts or DNA at the pregnancy-related state-associated loci), proteomic data comprising quantitative measures of proteins of the dataset at a panel of pregnancy-related state-associated proteins, and/or metabolome data comprising quantitative measures of a panel of pregnancy-related state-associated metabolites decreased from the earlier time point to the later time point), then the difference may be indicative of the subject having a decreased risk of the pregnancy-related state. A clinical action or decision may be made based on this indication of the decreased risk of the pregnancy-related state (e.g., continuing or ending a current therapeutic intervention) for the subject. The clinical action or decision may comprise recommending the subject for a secondary clinical test to confirm the decreased risk of the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

In some embodiments, a difference in the quantitative measures of sequence reads of the dataset at a panel of pregnancy-related state-associated loci (e.g., quantitative measures of RNA transcripts or DNA at the pregnancy-related state-associated loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of an efficacy of the course of treatment for treating the pregnancy-related state of the subject. For example, if the pregnancy-related state was detected in the subject at an earlier time point but was not detected in the subject at a later time point, then the difference may be indicative of an efficacy of the course of treatment for treating the pregnancy-related state of the subject. A clinical action or decision may be made based on this indication of the efficacy of the course of treatment for treating the pregnancy-related state of the subject, e.g., continuing or ending a current therapeutic intervention for the subject. The clinical action or decision may comprise recommending the subject for a secondary clinical test to confirm the efficacy of the course of treatment for treating the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

In some embodiments, a difference in the quantitative measures of sequence reads of the dataset at a panel of pregnancy-related state-associated loci (e.g., quantitative measures of RNA transcripts or DNA at the pregnancy-related state-associated loci), proteomic data comprising quantitative measures of proteins of the dataset at a panel of pregnancy-related state-associated proteins, and/or metabolome data comprising quantitative measures of a panel of pregnancy-related state-associated metabolites determined between the two or more time points may be indicative of a non-efficacy of the course of treatment for treating the pregnancy-related state of the subject. For example, if the pregnancy-related state was detected in the subject both at an earlier time point and at a later time point, and if the difference is a negative or zero difference (e.g., the quantitative measures of sequence reads of the dataset at a panel of pregnancy-related state-associated loci (e.g., quantitative measures of RNA transcripts or DNA at the pregnancy-related state-associated loci), proteomic data comprising quantitative measures of proteins of the dataset at a panel of pregnancy-related state-associated proteins, and/or metabolome data comprising quantitative measures of a panel of pregnancy-related state-associated metabolites increased or remained at a constant level from the earlier time point to the later time point), and if an efficacious treatment was indicated at an earlier time point, then the difference may be indicative of a non-efficacy of the course of treatment for treating the pregnancy-related state of the subject. A clinical action or decision may be made based on this indication of the non-efficacy of the course of treatment for treating the pregnancy-related state of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the pregnancy-related state. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, a cell-free biological cytology, an amniocentesis, a non-invasive prenatal test (NIPT), or any combination thereof.

In another aspect, the present disclosure provides a computer-implemented method for predicting a risk of pre-term birth of a subject, comprising: (a) receiving clinical health data of the subject, wherein the clinical health data comprises a plurality of quantitative or categorical measures of the subject; (b) using a trained algorithm to process the clinical health data of the subject to determine a risk score indicative of the risk of pre-term birth of the subject; and (c) electronically outputting a report indicative of the risk score indicative of the risk of pre-term birth of the subject.

In some embodiments, for example, the clinical health data comprises one or more quantitative measures of the subject, such as age, weight, height, body mass index (BMI), blood pressure, heart rate, glucose levels, number of previous pregnancies, and number of previous births. As another example, the clinical health data may comprise one or more categorical measures, such as race, ethnicity, history of medication or other clinical treatment, history of tobacco use, history of alcohol consumption, daily activity or fitness level, genetic test results, blood test results, imaging results, and fetal screening results.

In some embodiments, the computer-implemented method for predicting a risk of pre-term birth of a subject is performed using a computer or mobile device application. For example, a subject may use a computer or mobile device application to input her own clinical health data, including quantitative and/or categorical measures. The computer or mobile device application may then use a trained algorithm to process the clinical health data to determine a risk score indicative of the risk of pre-term birth of the subject. The computer or mobile device application may then display a report indicative of the risk score indicative of the risk of pre-term birth of the subject.

In some embodiments, the risk score indicative of the risk of pre-term birth of the subject may be refined by performing one or more subsequent clinical tests for the subject. For example, the subject may be referred by a physician for one or more subsequent clinical tests (e.g., an ultrasound imaging or a blood test) based on the initial risk score. Next, the computer or mobile device application may process results from the one or more subsequent clinical tests using a trained algorithm to determine an updated risk score indicative of the risk of pre-term birth of the subject.

In some embodiments, the risk score comprises a likelihood of the subject having a pre-term birth within a pre-determined duration of time. For example, the pre-determined duration of time may be about 1 hour, about 2 hours, about 4 hours, about 6 hours, about 8 hours, about 10 hours, about 12 hours, about 14 hours, about 16 hours, about 18 hours, about 20 hours, about 22 hours, about 24 hours, about 1.5 days, about 2 days, about 2.5 days, about 3 days, about 3.5 days, about 4 days, about 4.5 days, about 5 days, about 5.5 days, about 6 days, about 6.5 days, about 7 days, about 8 days, about 9 days, about 10 days, about 12 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 13 weeks, or more than about 13 weeks.

Outputting a Report of the Pregnancy-Related State

After the pregnancy-related state is identified or an increased risk of the pregnancy-related state is monitored in the subject, a report may be electronically outputted that is indicative of (e.g., identifies or provides an indication of) the pregnancy-related state of the subject. The subject may not display a pregnancy-related state (e.g., is asymptomatic of the pregnancy-related state such as a pregnancy-related complication). The report may be presented on a graphical user interface (GUI) of an electronic device of a user. The user may be the subject, a caretaker, a physician, a nurse, or another health care worker.

The report may include one or more clinical indications such as (i) a diagnosis of the pregnancy-related state of the subject, (ii) a prognosis of the pregnancy-related state of the subject, (iii) an increased risk of the pregnancy-related state of the subject, (iv) a decreased risk of the pregnancy-related state of the subject, (v) an efficacy of the course of treatment for treating the pregnancy-related state of the subject, and (vi) a non-efficacy of the course of treatment for treating the pregnancy-related state of the subject. The report may include one or more clinical actions or decisions made based on these one or more clinical indications. Such clinical actions or decisions may be directed to therapeutic interventions, induction or inhibition of labor, or further clinical assessment or testing of the pregnancy-related state of the subject.

For example, a clinical indication of a diagnosis of the pregnancy-related state of the subject may be accompanied with a clinical action of prescribing a new therapeutic intervention for the subject. As another example, a clinical indication of an increased risk of the pregnancy-related state of the subject may be accompanied with a clinical action of prescribing a new therapeutic intervention or switching therapeutic interventions (e.g., ending a current treatment and prescribing a new treatment) for the subject. As another example, a clinical indication of a decreased risk of the pregnancy-related state of the subject may be accompanied with a clinical action of continuing or ending a current therapeutic intervention for the subject. As another example, a clinical indication of an efficacy of the course of treatment for treating the pregnancy-related state of the subject may be accompanied with a clinical action of continuing or ending a current therapeutic intervention for the subject. As another example, a clinical indication of a non-efficacy of the course of treatment for treating the pregnancy-related state of the subject may be accompanied with a clinical action of ending a current therapeutic intervention and/or switching to (e.g., prescribing) a different new therapeutic intervention for the subject.

Computer Systems

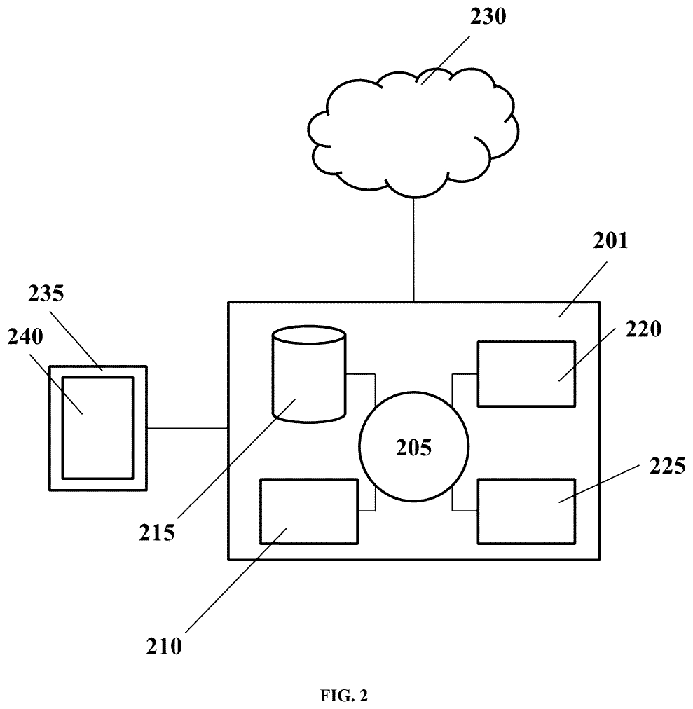

The present disclosure provides computer systems that are programmed to implement methods of the disclosure. FIG. 2 shows a computer system 201 that is programmed or otherwise configured to, for example, (i) train and test a trained algorithm, (ii) use the trained algorithm to process data to determine a pregnancy-related state of a subject, (iii) determine a quantitative measure indicative of a pregnancy-related state of a subject, (iv) identify or monitor the pregnancy-related state of the subject, and (v) electronically output a report that indicative of the pregnancy-related state of the subject.

The computer system 201 may regulate various aspects of analysis, calculation, and generation of the present disclosure, such as, for example, (i) training and testing a trained algorithm, (ii) using the trained algorithm to process data to determine a pregnancy-related state of a subject, (iii) determining a quantitative measure indicative of a pregnancy-related state of a subject, (iv) identifying or monitoring the pregnancy-related state of the subject, and (v) electronically outputting a report that indicative of the pregnancy-related state of the subject. The computer system 201 may be an electronic device of a user or a computer system that is remotely located with respect to the electronic device. The electronic device may be a mobile electronic device.

The computer system 201 includes a central processing unit (CPU, also “processor” and “computer processor” herein) 205, which may be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 may be a data storage unit (or data repository) for storing data. The computer system 201 may be operatively coupled to a computer network (“network”) 230 with the aid of the communication interface 220. The network 230 may be the Internet, an internet and/or extranet, or an intranet and/or extranet that is in communication with the Internet.

The network 230 in some cases is a telecommunication and/or data network. The network 230 may include one or more computer servers, which may enable distributed computing, such as cloud computing. For example, one or more computer servers may enable cloud computing over the network 230 (“the cloud”) to perform various aspects of analysis, calculation, and generation of the present disclosure, such as, for example, (i) training and testing a trained algorithm, (ii) using the trained algorithm to process data to determine a pregnancy-related state of a subject, (iii) determining a quantitative measure indicative of a pregnancy-related state of a subject, (iv) identifying or monitoring the pregnancy-related state of the subject, and (v) electronically outputting a report that indicative of the pregnancy-related state of the subject. Such cloud computing may be provided by cloud computing platforms such as, for example, Amazon Web Services (AWS), Microsoft Azure, Google Cloud Platform, and IBM cloud. The network 230, in some cases with the aid of the computer system 201, may implement a peer-to-peer network, which may enable devices coupled to the computer system 201 to behave as a client or a server.

The CPU 205 may comprise one or more computer processors and/or one or more graphics processing units (GPUs). The CPU 205 may execute a sequence of machine-readable instructions, which may be embodied in a program or software. The instructions may be stored in a memory location, such as the memory 210. The instructions may be directed to the CPU 205, which may subsequently program or otherwise configure the CPU 205 to implement methods of the present disclosure. Examples of operations performed by the CPU 205 may include fetch, decode, execute, and writeback.

The CPU 205 may be part of a circuit, such as an integrated circuit. One or more other components of the system 201 may be included in the circuit. In some cases, the circuit is an application specific integrated circuit (ASIC).

The storage unit 215 may store files, such as drivers, libraries and saved programs. The storage unit 215 may store user data, e.g., user preferences and user programs. The computer system 201 in some cases may include one or more additional data storage units that are external to the computer system 201, such as located on a remote server that is in communication with the computer system 201 through an intranet or the Internet.

The computer system 201 may communicate with one or more remote computer systems through the network 230. For instance, the computer system 201 may communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 201 via the network 230.

Methods as described herein may be implemented by way of machine (e.g., computer processor) executable code stored on an electronic storage location of the computer system 201, such as, for example, on the memory 210 or electronic storage unit 215. The machine executable or machine readable code may be provided in the form of software. During use, the code may be executed by the processor 205. In some cases, the code may be retrieved from the storage unit 215 and stored on the memory 210 for ready access by the processor 205. In some situations, the electronic storage unit 215 may be precluded, and machine-executable instructions are stored on memory 210.

The code may be pre-compiled and configured for use with a machine having a processer adapted to execute the code, or may be compiled during runtime. The code may be supplied in a programming language that may be selected to enable the code to execute in a pre-compiled or as-compiled fashion.

Aspects of the systems and methods provided herein, such as the computer system 201, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 may include or be in communication with an electronic display 235 that comprises a user interface (UI) 240 for providing, for example, (i) a visual display indicative of training and testing of a trained algorithm, (ii) a visual display of data indicative of a pregnancy-related state of a subject, (iii) a quantitative measure of a pregnancy-related state of a subject, (iv) an identification of a subject as having a pregnancy-related state, or (v) an electronic report indicative of the pregnancy-related state of the subject. Examples of UIs include, without limitation, a graphical user interface (GUI) and web-based user interface.

Methods and systems of the present disclosure may be implemented by way of one or more algorithms. An algorithm may be implemented by way of software upon execution by the central processing unit 205. The algorithm can, for example, (i) train and test a trained algorithm, (ii) use the trained algorithm to process data to determine a pregnancy-related state of a subject, (iii) determine a quantitative measure indicative of a pregnancy-related state of a subject, (iv) identify or monitor the pregnancy-related state of the subject, and (v) electronically output a report that indicative of the pregnancy-related state of the subject.

### EXAMPLES

### Example 1: Cohort of Subject and Methodology

Using systems and methods of the present disclosure, a method of detection and measurement of the pregnancy RNA transcriptional and DNA methylation signals in maternal plasma were developed to detect pregnancy at an early stage and monitor pregnancy progression and health.

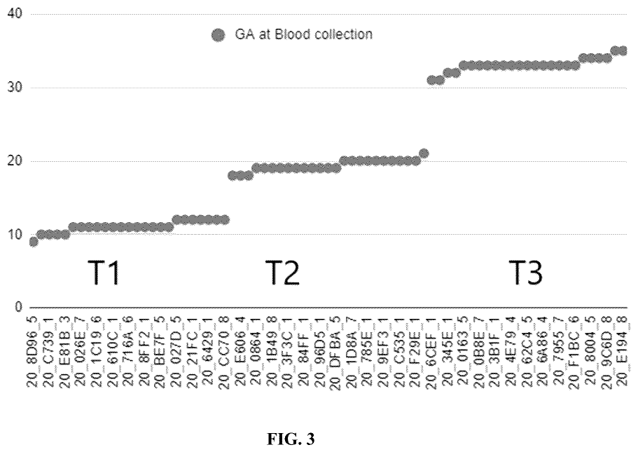

The cohort of subjects was obtained as follows. As shown in FIG. 3, a cohort of 90 subjects (15 non-pregnant and 75 healthy pregnant women) was established (with patient identification numbers shown on the x-axis). From this cohort, biological samples were collected and assayed at different time points corresponding to an estimated gestational age (shown on the y-axis, in increasing order of estimated gestational age) of a fetus of each subject, using methods and systems of the present disclosure. For example, the estimated gestational age (shown on the y-axis) may be determined using methods such as ultrasound imaging, a last menstrual period (LMP) date, or a combination thereof, and may range from 0 to about 36 weeks.

Table 1 shows a number of collected samples for non-pregnant and pregnant groups per trimester at the time of collection of each sample.

TABLE 1
Number of collected samples for non-pregnant and pregnant groups
per trimester at the time of collection of each sample

| Sample type | Number of samples | Blood collection GA range |
|---|---|---|
| Non-pregnant | 15 | NA |
| first trimester | 25 | 9-12 weeks |
| second trimester | 25 | 18-21 weeks |
| third trimester | 25 | 31-35 weeks |

Blood was collected into Streck Total RNA blood tubes, and plasma was separated from the blood samples after centrifugation at 1,500×g for 15 minutes at room temperature followed by 2,000×g at 15 minutes. The cell-free DNA (cfDNA) and cell-free RNA (cfRNA) were extracted from the plasma of each sample.

Whole transcriptional analyses of cfRNA were performed by sequencing. About 20,000 genes with a median expression greater than zero were used for RNA expression analysis.

DNA methylation states in each cfDNA were measured by performing a whole-genome cell-free 5hmC sequencing method based on selective chemical labeling (hMe-Seal) (as described by, for example, Song et al., “5-Hydroxymethylcytosine signatures in cell-free DNA provide information about tumor types and stages”, Cell Research, 27, 1231-1242, 2017, which is incorporated by reference herein in its entirety). A set of 4,357,876 methylation features are distributed in 11 feature classes. Table 2 shows the feature class types of DNA methylation changes associated with 5hmC based on mapping to the whole genome.

TABLE 2
Feature class types of DNA methylation changes associated
with 5 hmC based on mapping to the whole genome

| | Number of | |
|---|---|---|
| Feature | features | |
| class | in class | Class description |
| SINE | 1,707,593 | Short-interspersed nuclear elements; non-autonomous, non-coding |
| | | transposable, 100-700 bp long |
| LINE | 1,473,289 | Long-interspersed nuclear elements; non-long-terminal-repeat |
| | | retrotransposons, ~7,000 bp long |
| Exon | 517,425 | Part of gene encoding a part of the mature (post-spliced) RNA |
| | | molecule, ~300 bp |
| Intron | 226,801 | Intragenic region removed from pre-mRNA by splicing, ~6,400 bp |
| 5′-UTR | 99,522 | 5′-untranslated region; a sequence upstream of the initiation codon, |
| | | transcribed into mature mRNA, ~150 bp |
| 3′-UTR | 115,547 | 3′-untranslated region; a sequence immediately downstream of the |
| | | stop codon, transcribed into mature mRNA, ~520 bp |
| CTCF | 78,824 | Binding site of the ubiquitous transcriptional chromatin regulator |
| | | CTCF, 14 bp |
| CpG- | 56,952 | Regions with high frequency of CpG dinucleotides, frequently |
| island | | colocalizing with promoters, 300-3,000 bp |
| Promoter | 31,871 | Transcription initiators' binding site proximal to genes, ~600 bp |
| Enhancer | 30,614 | Transcription activators' binding site distal from genes, 50-1500 bp |
| Gene | 19,438 | ~28,000 bp |

### Example 2: Detection DNA Methylation State Changes for Separating First Trimester Pregnant Vs Nonpregnant Subjects

Blood samples collected at the first trimester of pregnancy (25 subjects) and non-pregnant samples (15 subjects) were used to determine the DNA methylation state changes associated with early pregnancy.

In the first analysis, only gene DNA methylation class of 19,438 features were used for differential analysis between non-pregnant and pregnant samples. A set of 3,694 genes were determined to be significantly differentially methylated between first trimester and non-pregnant samples. Table 3 shows the top 100 differentially methylated genes between non-pregnant and first trimester pregnancy samples.

TABLE 3
Top 100 most differentially methylated genes between
first trimester pregnant and non-pregnant samples

| | | Adjusted | % of maximum |
|---|---|---|---|
| Gene | W-statistic | p-value | misclassification |
| ADAM18 | 0 | 7.79E−07 | 0 |
| ANKRD30B | 0 | 7.79E−07 | 0 |
| ERVV-2 | 0 | 7.79E−07 | 0 |
| FBN2 | 0 | 7.79E−07 | 0 |
| IGSF5 | 0 | 7.79E−07 | 0 |
| LZTS1 | 0 | 7.79E−07 | 0 |
| OR5H14 | 0 | 7.79E−07 | 0 |
| PSG1 | 0 | 7.79E−07 | 0 |
| PSG2 | 0 | 7.79E−07 | 0 |
| SCNN1B | 0 | 7.79E−07 | 0 |
| SPAM1 | 0 | 7.79E−07 | 0 |
| VGLL3 | 0 | 7.79E−07 | 0 |
| ZFP42 | 0 | 7.79E−07 | 0 |
| ZNF675 | 0 | 7.79E−07 | 0 |
| C5orf38 | 1 | 1.21E−06 | 3 |
| CLMP | 1 | 1.21E−06 | 3 |
| HUNK | 1 | 1.21E−06 | 3 |
| ZIM2 | 1 | 1.21E−06 | 3 |
| ABCA12 | 2 | 1.41E−06 | 5 |
| ATP6V1C2 | 2 | 1.41E−06 | 5 |
| DDB1 | 2 | 1.41E−06 | 5 |
| GRHL2 | 2 | 1.41E−06 | 5 |
| LAMA1 | 2 | 1.41E−06 | 5 |
| NEURL1 | 2 | 1.41E−06 | 5 |
| PKIB | 2 | 1.41E−06 | 5 |
| SERPINB7 | 2 | 1.41E−06 | 5 |
| TENM3 | 2 | 1.41E−06 | 5 |
| TFAP2C | 2 | 1.41E−06 | 5 |
| ZNF573 | 2 | 1.41E−06 | 5 |
| ZNF578 | 2 | 1.41E−06 | 5 |
| ZNF701 | 2 | 1.41E−06 | 5 |
| AC006538.1 | 3 | 1.82E−06 | 8 |
| ARHGEF16 | 3 | 1.82E−06 | 8 |
| FUT9 | 3 | 1.82E−06 | 8 |
| GPC1 | 3 | 1.82E−06 | 8 |
| ISL1 | 3 | 1.82E−06 | 8 |
| LIFR | 3 | 1.82E−06 | 8 |
| TRIML2 | 3 | 1.82E−06 | 8 |
| VSX2 | 3 | 1.82E−06 | 8 |
| WDR64 | 3 | 1.82E−06 | 8 |
| ZNF331 | 3 | 1.82E−06 | 8 |
| ZNF761 | 3 | 1.82E−06 | 8 |
| ACER2 | 4 | 2.34E−06 | 11 |
| ANKRD30A | 4 | 2.34E−06 | 11 |
| CELSR1 | 4 | 2.34E−06 | 11 |
| CFAP300 | 4 | 2.34E−06 | 11 |
| COBLL1 | 4 | 2.34E−06 | 11 |
| NLRP10 | 4 | 2.34E−06 | 11 |
| NPSR1 | 4 | 2.34E−06 | 11 |
| PEG3 | 4 | 2.34E−06 | 11 |
| PSG3 | 4 | 2.34E−06 | 11 |
| PSG7 | 4 | 2.34E−06 | 11 |
| SLC27A6 | 4 | 2.34E−06 | 11 |
| SMOC2 | 4 | 2.34E−06 | 11 |
| TCF25 | 4 | 2.34E−06 | 11 |
| ZNF354B | 4 | 2.34E−06 | 11 |
| AC007846.2 | 5 | 3.00E−06 | 14 |
| BCAR1 | 5 | 3.00E−06 | 14 |
| BCAR3 | 5 | 3.00E−06 | 14 |
| CYP4B1 | 5 | 3.00E−06 | 14 |
| DLG5 | 5 | 3.00E−06 | 14 |
| EFEMP1 | 5 | 3.00E−06 | 14 |
| EFHD1 | 5 | 3.00E−06 | 14 |
| EGFR | 5 | 3.00E−06 | 14 |
| PAX1 | 5 | 3.00E−06 | 14 |
| PCP4 | 5 | 3.00E−06 | 14 |
| PSG6 | 5 | 3.00E−06 | 14 |
| SPATA3 | 5 | 3.00E−06 | 14 |
| TUSC3 | 5 | 3.00E−06 | 14 |
| ADAMTS6 | 6 | 4.09E−06 | 16 |
| EPAS1 | 6 | 4.09E−06 | 16 |
| EYA2 | 6 | 4.09E−06 | 16 |
| GCM1 | 6 | 4.09E−06 | 16 |
| GRHL1 | 6 | 4.09E−06 | 16 |
| GRM6 | 6 | 4.09E−06 | 16 |
| ILVBL | 6 | 4.09E−06 | 16 |
| PSG11 | 6 | 4.09E−06 | 16 |
| URB2 | 6 | 4.09E−06 | 16 |
| ZNF525 | 6 | 4.09E−06 | 16 |
| ZNF83 | 6 | 4.09E−06 | 16 |
| AC010463.1 | 7 | 5.45E−06 | 19 |
| ARHGAP28 | 7 | 5.45E−06 | 19 |
| DENND2A | 7 | 5.45E−06 | 19 |
| DPRX | 7 | 5.45E−06 | 19 |
| ESRRG | 7 | 5.45E−06 | 19 |
| LCMT1 | 7 | 5.45E−06 | 19 |
| NRK | 7 | 5.45E−06 | 19 |
| PALLD | 7 | 5.45E−06 | 19 |
| PSG8 | 7 | 5.45E−06 | 19 |
| ZNF28 | 7 | 5.45E−06 | 19 |
| ADAM2 | 8 | 7.38E−06 | 22 |
| CLIP1 | 8 | 7.38E−06 | 22 |
| GPC3 | 8 | 7.38E−06 | 22 |
| IRX2 | 8 | 7.38E−06 | 22 |
| MARVELD3 | 8 | 7.38E−06 | 22 |
| PLXNA1 | 8 | 7.38E−06 | 22 |
| PPARD | 8 | 7.38E−06 | 22 |
| SPIRE2 | 8 | 7.38E−06 | 22 |
| TLL1 | 8 | 7.38E−06 | 22 |
| AC022137.3 | 9 | 9.36E−06 | 24 |

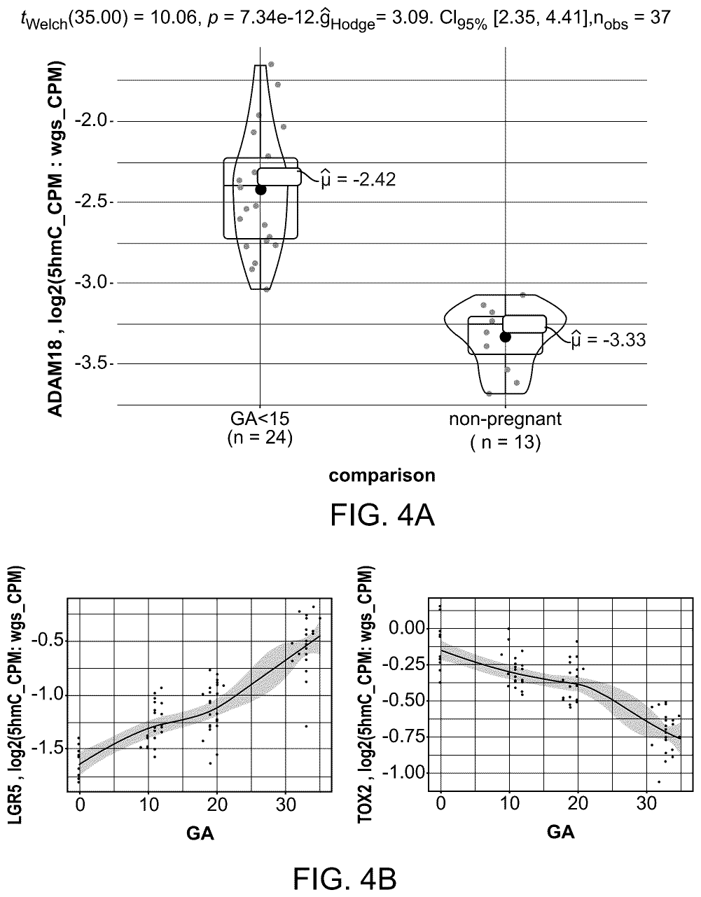

14 gene features fully discriminated (W=0, Percent misclassification=0) between the samples from non-pregnant women and first trimester samples. FIG. 4A shows an example of ADAM18, a fully discriminating gene feature. As shown in the figure, there was complete separation between samples from 13 non-pregnant subjects and 24 samples from pregnant subjects in the first trimester (gestational age <15 weeks), based on 5hmC DNA methylation profiling using the ADAM18 gene.

In the second analysis, only all non-gene DNA methylation features were used for differential analysis between non-pregnant and pregnant samples. More than 10,000 non-genic features, excluding genes, introns, exons, were determined to be significantly differentially methylated between non-pregnant and first trimester pregnancy samples. Of those, 21 non-genic features listed in Table 4 fully discriminated between pregnant and non-pregnant samples.

TABLE 4
21 differentially DNA methylated non-genic features between
non-pregnant and first trimester pregnancy samples

| | | Adjusted | Feature |
|---|---|---|---|
| Feature name | W-statistic | p-value | class |
| AluSq4_13203436_288 | 0 | 1.12E−04 | SINE |
| AluSx4_62013684_294 | 0 | 1.12E−04 | SINE |
| LIPA5_126343046_4528 | 0 | 2.25E−04 | LINE |
| LIPA8A_128455512_6469 | 0 | 2.25E−04 | LINE |
| LIMA2_43265802_2517 | 0 | 1.53E−04 | LINE |
| OR5H14_98150317_6296 | 0 | 3.24E−05 | 3′-UTR |
| PSG11_43018768_279 | 0 | 3.24E−05 | 3′-UTR |
| CpG-21928 | 0 | 1.07E−05 | CpG-island |
| CpG-21928_1111387 | 0 | 1.07E−05 | CpG-island |
| CpG-21930 | 0 | 1.07E−05 | CpG-island |
| pcawg_314 | 0 | 1.72E−05 | enhancer |
| DLX6-AS1_promoter | 0 | 1.79E−06 | promoter |
| DPRX_promoter | 0 | 1.79E−06 | promoter |
| IGSF5_promoter | 0 | 1.79E−06 | promoter |
| MIR1283-1_promoter | 0 | 1.79E−06 | promoter |
| MIR518B_promoter | 0 | 1.79E−06 | promoter |
| MIR518E_promoter | 0 | 1.79E−06 | promoter |
| MIR518F_promoter | 0 | 1.79E−06 | promoter |
| MIR520C_promoter | 0 | 1.79E−06 | promoter |

### Example 3: Detection DNA Methylation State Change Across Pregnancy

A gestational age cohort of subjects (non-pregnant and pregnant women) was established, from which one or more biological samples (e.g., 1 or 2 each) were collected and assayed at different time points corresponding to an estimated gestational age of a fetus of each subject, using methods and systems of the present disclosure. The cohort used for analysis included all 90 subjects as described in Example 1.

In the first analysis, only gene DNA methylation class of 19,438 features were used for differential analysis between non-pregnant and pregnant samples collected at first, second, and third trimesters. A set of 3870 genes was determined to be significantly differentially methylated across pregnancy. A set of 2522 genes was determined to show significant increase in 5hmC DNA methylation state changes and 1348 genes decrease in 5hmC DNA methylation state across pregnancy. Table 5 presents (in decreasing order of the absolute magnitude of a slope) top 100 genes with the largest upward and top 100 genes with the largest downward DNA methylation trends across the pregnancy.

TABLE 5
Top 100 genes with the largest upward (left 3 columns) and top 100 genes with
the largest downward (right 3 columns) DNA methylation trends across pregnancy

| | Slope, | | | Slope, | |
|---|---|---|---|---|---|
| | increase | | | decrease | |
| | in 5 hmC | Bonferroni | | in 5 hmC | Bonferroni |
| | fraction | adjusted | | fraction | adjusted |
| Gene | per week | p-value | Gene | per week | p-value |
| C5orf38 | 0.07 | 1.41E−15 | CEACAM3 | −0.03 | 1.31E−06 |
| OR5H14 | 0.07 | 6.28E−17 | FCN1 | −0.03 | 2.88E−06 |
| ZFP42 | 0.07 | 2.77E−16 | CEACAM8 | −0.03 | 7.92E−04 |
| KRTAP26-1 | 0.06 | 5.89E−08 | PI3 | −0.03 | 1.34E−03 |
| FTMT | 0.06 | 4.74E−11 | LILRA2 | −0.03 | 5.31E−08 |
| IRX2 | 0.06 | 7.48E−16 | CEACAM4 | −0.03 | 7.32E−05 |
| ERVV-2 | 0.06 | 3.75E−15 | CEACAM6 | −0.03 | 1.44E−06 |
| PAX1 | 0.06 | 1.01E−17 | CTSG | −0.02 | 2.94E−03 |
| PSG2 | 0.06 | 1.39E−15 | MNDA | −0.02 | 4.46E−06 |
| SPRR3 | 0.06 | 1.14E−10 | HLA-A | −0.02 | 5.22E−09 |
| IGSF5 | 0.06 | 2.85E−18 | RNASE2 | −0.02 | 9.24E−04 |
| PSG1 | 0.06 | 4.09E−15 | TLR4 | −0.02 | 6.43E−09 |
| ERVV-1 | 0.06 | 2.46E−08 | SIGLEC9 | −0.02 | 3.78E−09 |
| MUC15 | 0.06 | 3.88E−16 | LILRB2 | −0.02 | 2.80E−06 |
| PSG6 | 0.06 | 6.35E−15 | LILRA1 | −0.02 | 7.85E−07 |
| LGALS16 | 0.05 | 4.76E−13 | GPR4 | −0.02 | 4.32E−08 |
| CLDN8 | 0.05 | 2.00E−07 | LAIR1 | −0.02 | 4.43E−09 |
| FBN2 | 0.05 | 2.11E−21 | GZMB | −0.02 | 5.25E−03 |
| TENM3 | 0.05 | 2.18E−20 | KIR2DL3 | −0.02 | 1.06E−05 |
| TRIML2 | 0.05 | 1.86E−13 | LILRA4 | −0.02 | 1.91E−08 |
| PSG11 | 0.05 | 3.25E−14 | LILRA5 | −0.02 | 2.88E−03 |
| ANKRD30B | 0.05 | 3.43E−17 | RAC2 | −0.02 | 7.68E−13 |
| MSX2 | 0.05 | 1.48E−20 | KIR2DL1 | −0.02 | 1.20E−08 |
| SPAM1 | 0.05 | 9.40E−18 | CYTH4 | −0.02 | 3.71E−14 |
| TFAP2B | 0.05 | 1.02E−15 | TYROBP | −0.02 | 3.65E−05 |
| PEG3 | 0.05 | 1.38E−16 | FCAR | −0.02 | 1.01E−05 |
| SCYGR7 | 0.05 | 5.01E−04 | CD300LB | −0.02 | 3.49E−06 |
| SLC19A3 | 0.05 | 6.72E−15 | HRH2 | −0.02 | 1.01E−07 |
| DPRX | 0.05 | 4.03E−12 | HLA-DMB | −0.02 | 3.36E−09 |
| HIST1H4E | 0.05 | 7.35E−05 | KIR3DL1 | −0.02 | 6.94E−07 |
| ZIM2 | 0.05 | 9.34E−18 | LILRB1 | −0.02 | 3.74E−10 |
| KRTAP2-2 | 0.05 | 7.25E−04 | CYP4F3 | −0.02 | 3.52E−04 |
| APELA | 0.05 | 1.82E−14 | RFLNB | −0.02 | 2.37E−05 |
| VGLL3 | 0.05 | 1.67E−20 | ZNF516 | −0.02 | 3.86E−15 |
| HIST1H2BH | 0.05 | 8.49E−08 | HLA-DRA | −0.02 | 7.57E−07 |
| TFAP2C | 0.05 | 1.30E−15 | MMP9 | −0.02 | 1.17E−04 |
| TMEM229A | 0.05 | 2.14E−05 | ETS1 | −0.02 | 3.03E−18 |
| KRTAP19-5 | 0.05 | 8.04E−03 | MS4A6A | −0.02 | 3.41E−06 |
| KRTAP19-1 | 0.05 | 3.05E−03 | GIMAP8 | −0.02 | 2.00E−11 |
| FAM83B | 0.05 | 1.46E−15 | FFAR2 | −0.02 | 1.85E−05 |
| KRTAP2-3 | 0.05 | 1.72E−04 | LDB2 | −0.02 | 4.78E−06 |
| ADAM18 | 0.05 | 3.87E−18 | TNF | −0.02 | 5.16E−04 |
| ISL1 | 0.05 | 6.37E−09 | TOR4A | −0.02 | 2.58E−06 |
| LGALS13 | 0.05 | 5.96E−13 | ABCA13 | −0.02 | 1.11E−08 |
| XAGE5 | 0.05 | 3.81E−14 | Z82206.1 | −0.02 | 7.09E−04 |
| TUSC3 | 0.04 | 9.44E−15 | LILRA6 | −0.02 | 1.30E−04 |
| PSG5 | 0.04 | 6.88E−13 | OR10J1 | −0.02 | 1.55E−03 |
| NLRP10 | 0.04 | 1.26E−09 | CLEC5A | −0.02 | 4.48E−08 |
| PSG7 | 0.04 | 5.39E−11 | MMP8 | −0.02 | 3.96E−03 |
| GABRE | 0.04 | 2.77E−17 | GLT1D1 | −0.02 | 2.27E−03 |
| NRK | 0.04 | 5.81E−20 | GIMAP1 | −0.02 | 2.19E−08 |
| PSG3 | 0.04 | 2.34E−14 | JAML | −0.02 | 2.11E−09 |
| KPRP | 0.04 | 6.31E−12 | TMEM273 | −0.02 | 1.96E−11 |
| ABCA12 | 0.04 | 3.02E−22 | CXCR4 | −0.02 | 6.31E−03 |
| KRT34 | 0.04 | 5.53E−14 | CD1B | −0.02 | 8.16E−03 |
| OPRK1 | 0.04 | 5.63E−19 | MPZL3 | −0.02 | 2.93E−10 |
| DLX5 | 0.04 | 2.17E−10 | CD33 | −0.02 | 1.71E−07 |
| CAPN6 | 0.04 | 2.22E−15 | NCR3 | −0.02 | 5.30E−05 |
| DLX6 | 0.04 | 3.12E−11 | GYPC | −0.02 | 1.41E−11 |
| ODAM | 0.04 | 4.34E−08 | AL160272.2 | −0.02 | 1.39E−11 |
| TMPRSS11F | 0.04 | 1.24E−20 | CLEC17A | −0.02 | 1.02E−08 |
| CFAP300 | 0.04 | 2.16E−16 | SAMSN1 | −0.02 | 1.07E−12 |
| ARMS2 | 0.04 | 9.29E−09 | CD300E | −0.02 | 1.69E−07 |
| EFEMP1 | 0.04 | 6.41E−17 | GIMAP1- | −0.02 | 3.77E−11 |
| | | | GIMAP5 | | |
| HSD3B1 | 0.04 | 6.74E−12 | SIGLEC10 | −0.02 | 2.41E−07 |
| HIST1H3H | 0.04 | 2.57E−05 | AL645941.2 | −0.02 | 3.19E−13 |
| SVEP1 | 0.04 | 1.71E−23 | CD300A | −0.02 | 9.73E−12 |
| LGALS14 | 0.04 | 4.82E−12 | MYO1F | −0.02 | 3.21E−08 |
| TBX3 | 0.04 | 2.59E−10 | ADGRE3 | −0.02 | 5.93E−12 |
| DLX4 | 0.04 | 6.75E−14 | TNFSF14 | −0.02 | 3.52E−09 |
| LGSN | 0.04 | 1.16E−15 | LILRB5 | −0.02 | 1.39E−06 |
| ZSCAN4 | 0.04 | 1.12E−12 | CERS3 | −0.02 | 2.60E−05 |
| TLL1 | 0.04 | 8.08E−18 | RASA3 | −0.02 | 9.04E−12 |
| HIST3H2A | 0.04 | 6.08E−03 | SIGLEC7 | −0.02 | 1.16E−05 |
| ZNF90 | 0.04 | 7.71E−15 | RNF166 | −0.02 | 1.34E−07 |
| KRT24 | 0.04 | 2.91E−15 | VSTM1 | −0.02 | 2.78E−04 |
| VGLL2 | 0.04 | 2.17E−11 | KIR2DL4 | −0.02 | 3.80E−05 |
| CCK | 0.04 | 4.45E−13 | LTF | −0.02 | 4.79E−05 |
| BARX2 | 0.04 | 1.69E−20 | LCP2 | −0.02 | 1.86E−09 |
| AL354822.1 | 0.04 | 5.83E−04 | TOX2 | −0.02 | 2.86E−18 |
| FZD10 | 0.04 | 1.23E−05 | LTB4R | −0.02 | 2.51E−05 |
| NR2F2 | 0.04 | 1.15E−14 | PRSS33 | −0.02 | 1.90E−05 |
| WDR64 | 0.04 | 6.78E−18 | DACH1 | −0.02 | 1.33E−11 |
| OR5H1 | 0.04 | 8.96E−08 | CEACAM21 | −0.02 | 1.27E−08 |
| CCDC201 | 0.04 | 8.10E−18 | MBP | −0.02 | 4.13E−15 |
| TACC2 | 0.04 | 1.19E−15 | OOSP1 | −0.02 | 8.65E−09 |
| TBX20 | 0.04 | 1.18E−15 | LYPD4 | −0.02 | 7.49E−05 |
| DSC3 | 0.04 | 2.67E−17 | A4GNT | −0.02 | 1.73E−05 |
| PHYHIPL | 0.04 | 7.66E−14 | AGAP2 | −0.02 | 9.51E−09 |
| SPATA8 | 0.04 | 1.76E−06 | MICB | −0.02 | 3.84E−10 |
| BMP7 | 0.04 | 1.10E−19 | IL17RA | −0.02 | 1.56E−10 |
| IZUMO1R | 0.04 | 1.88E−06 | HK3 | −0.02 | 2.36E−05 |
| DACT2 | 0.04 | 9.30E−11 | CD300C | −0.02 | 8.47E−07 |
| YAP1 | 0.04 | 4.99E−12 | MS4A3 | −0.02 | 8.39E−04 |
| PSG8 | 0.04 | 6.62E−14 | FLRT2 | −0.02 | 2.85E−10 |
| TFPI2 | 0.04 | 6.37E−12 | CHST2 | −0.02 | 1.36E−05 |
| ESRRG | 0.04 | 2.84E−19 | AP4B1 | −0.02 | 1.58E−07 |
| SYT10 | 0.04 | 1.51E−18 | GIMAP5 | −0.02 | 5.23E−07 |
| PPP4R3C | 0.04 | 3.93E−03 | CD300LF | −0.02 | 5.00E−10 |
| DIRC1 | 0.04 | 2.59E−17 | SIRPD | −0.02 | 7.41E−04 |

FIG. 4B shows a representative upward trend indicative of 5hmC increase in DNA methylation state for the LGR5 gene locus (left), and a downward trend indicative of 5hmC decrease in DNA methylation state for the TOX2 gene locus (right).

In the second analysis, only all non-gene DNA methylation features were used for differential analysis between non-pregnant and pregnant samples collected at first, second, and third trimesters. A set of 17,085 non-genic DNA features were determined to be significantly differentially methylated across pregnancy. A set of 16,599 features were determined to show significant increase in 5hmC DNA methylation state changes and 476 features decrease in 5hmC DNA methylation state across pregnancy. Table 6 shows the top 100 non-genic features that showed an upward trend (increase in 5hmC DNA methylation state) and the top 100 non-genic features that showed a downward trend (decrease in 5hmC DNA methylation state) across pregnancy.

TABLE 6
Top 100 non-genic features with the largest upward DNA methylation trends (left 4 columns) and top 100
non-genic features with the largest downward DNA methylation trends (right 4 columns) across pregnancy

| | | Bonferroni | | | | Bonferroni | |
|---|---|---|---|---|---|---|---|
| | Slope, | adjusted | Feature | | Slope, | adjusted | Feature |
| Feature name | increase | p-value | class | Feature name | decrease | p-value | class |
| MIR3074_promoter | 0.02 | 1.91E−09 | Promoter | MIR6815_promoter | −0.02 | 1.88E−09 | Promoter |
| FRMD1_168053164_3931 | 0.03 | 6.50E−09 | 3′-UTR | CPNE6 promoter_2 | −0.02 | 6.39E−09 | Promoter |
| FRMD6-AS2_promoter | 0.04 | 9.98E−09 | Promoter | TLR4_117714647_10087 | −0.02 | 9.94E−09 | 3′-UTR |
| AluSx3_13237167_301 | 0.06 | 1.31E−08 | SINE | pcawg_15786 | −0.02 | 1.28E−08 | Enhancer |
| L1PA11_100383457_2454 | 0.05 | 1.53E−08 | LINE | GP1BB_promoter | −0.02 | 1.46E−08 | Promoter |
| SEMA6D_47770546_3675 | 0.05 | 1.55E−08 | 3′-UTR | SIGLEC9_promoter | −0.03 | 1.50E−08 | Promoter |
| BEAN1_promoter | 0.03 | 3.13E−08 | Promoter | GAS6-AS1_promoter | −0.02 | 2.91E−08 | Promoter |
| LINC02060_promoter | 0.04 | 3.16E−08 | Promoter | LINC00908_promoter | −0.02 | 3.01E−08 | Promoter |
| L2a_52323104_448 | 0.06 | 3.76E−08 | LINE | AOAH-IT1_promoter | −0.02 | 3.46E−08 | Promoter |
| pcawg_22705 | 0.05 | 5.56E−08 | Enhancer | CEACAM4_promoter_1 | −0.03 | 5.20E−08 | Promoter |
| LOC642648_promoter | 0.04 | 6.37E−08 | Promoter | CD300LB_promoter | −0.03 | 5.86E−08 | Promoter |
| MIR23B_promoter | 0.02 | 7.44E−08 | Promoter | MIR7850_promoter | −0.02 | 6.64E−08 | Promoter |
| FREM2_promoter | 0.04 | 7.68E−08 | Promoter | KCNAB2_promoter_3 | −0.02 | 6.89E−08 | Promoter |
| GAST_promoter | 0.02 | 9.68E−08 | Promoter | HELZ2_promoter_2 | −0.02 | 8.36E−08 | Promoter |
| FADS6_promoter | 0.02 | 1.06E−07 | Promoter | HGF_promoter | −0.02 | 9.89E−08 | Promoter |
| ARHGAP28_6788492_3177 | 0.04 | 1.30E−07 | 5'-UTR | GHET1_promoter | −0.02 | 1.21E−07 | Promoter |
| L1PB1_100649814_690 | 0.06 | 1.38E−07 | LINE | pcawg_5583 | −0.02 | 1.24E−07 | Enhancer |
| MPG_promoter | 0.02 | 2.62E−07 | Promoter | GIMAP1_promoter | −0.02 | 2.33E−07 | Promoter |
| AluSz6_34289208_281 | 0.06 | 2.73E−07 | SINE | CpG-18100 | −0.02 | 2.44E−07 | CpG |
| TMEM54_promoter | 0.02 | 3.26E−07 | Promoter | GIMAP1-GIMAP5_promoter | −0.02 | 2.93E−07 | Promoter |
| NOX4_promoter_2 | 0.04 | 3.88E−07 | Promoter | LAT_promoter | −0.02 | 3.48E−07 | Promoter |
| ZNF578_52512153_4728 | 0.03 | 5.00E−07 | 3′-UTR | LILRA2_promoter_2 | −0.03 | 4.45E−07 | Promoter |
| CpG-19219_1108678 | 0.07 | 5.64E−07 | CpG | GABBR1_promoter_1 | −0.02 | 4.89E−07 | Promoter |
| L2b_62885581_116 | 0.07 | 8.69E−07 | LINE | RNY1_promoter | −0.02 | 7.78E−07 | Promoter |
| VWA1_promoter | 0.02 | 9.03E−07 | Promoter | CACNG8_53982848_7366 | −0.02 | 8.11E−07 | 3′-UTR |
| CLPSL2_promoter | 0.02 | 1.25E−06 | Promoter | HLA-DRA_promoter | −0.02 | 1.14E−06 | Promoter |
| CpG-17912 | 0.05 | 1.32E−06 | CpG | CD300LD_promoter | −0.02 | 1.20E−06 | Promoter |
| TUFT1_151581706_1876 | 0.03 | 1.42E−06 | 3′-UTR | TRPM2_promoter_1 | −0.02 | 1.30E−06 | Promoter |
| AluSz6_60201706_307 | 0.06 | 1.43E−06 | SINE | ITGB2 promoter_2 | −0.02 | 1.30E−06 | Promoter |
| SEC31B_100507423_143 | 0.07 | 1.58E−06 | 3′-UTR | LILRA1_promoter_2 | −0.03 | 1.48E−06 | Promoter |
| CpG-9209 | 0.05 | 1.62E−06 | CpG | LAIR1_promoter_2 | −0.02 | 1.53E−06 | Promoter |
| pcawg_23678 | 0.05 | 1.86E−06 | Enhancer | S100B_promoter | −0.02 | 1.67E−06 | Promoter |
| AluJr4_91346845_280 | 0.07 | 2.20E−06 | SINE | KCNAB2_promoter_4 | −0.02 | 1.87E−06 | Promoter |
| DAAM2_promoter | 0.02 | 2.25E−06 | Promoter | TYROBP_promoter | −0.02 | 1.90E−06 | Promoter |
| FAM83B_54942006_3092 | 0.05 | 2.27E−06 | 3′-UTR | LILRA4_promoter | −0.02 | 1.96E−06 | Promoter |
| PDC_promoter_2 | 0.02 | 2.78E−06 | Promoter | MIR3186_promoter | −0.02 | 2.50E−06 | Promoter |
| MAP1LC3C_promoter | 0.02 | 2.84E−06 | Promoter | LOC102724163_promoter | −0.02 | 2.57E−06 | Promoter |
| AluSc8_105287329_271 | 0.07 | 3.71E−06 | SINE | PIK3CD-AS2_promoter | −0.02 | 3.41E−06 | Promoter |
| L1MB5_129739905_521 | 0.07 | 3.96E−06 | LINE | ITGAD_promoter | −0.02 | 3.57E−06 | Promoter |
| AluSc8_102211703_299 | 0.06 | 4.00E−06 | SINE | SIGLEC10_promoter | −0.02 | 3.57E−06 | Promoter |
| pcawg_15498 | 0.03 | 5.32E−06 | Enhancer | CRYBB1_promoter | −0.02 | 4.56E−06 | Promoter |
| ARHGEF10L_promoter_1 | 0.02 | 5.81E−06 | Promoter | LILRB2_promoter_1 | −0.03 | 4.94E−06 | Promoter |
| CpG-4759 | 0.04 | 6.01E−06 | CpG | KCNAB2_6097267_3736 | −0.02 | 5.20E−06 | 3′-UTR |
| PAPPA2_176463169_248 | 0.06 | 6.16E−06 | 5′-UTR | MIR4648_promoter | −0.02 | 5.48E−06 | Promoter |
| pcawg_20957 | 0.05 | 7.34E−06 | Enhancer | ADORA3_promoter | −0.02 | 6.64E−06 | Promoter |
| L1PA3_75727625_6152 | 0.03 | 7.96E−06 | LINE | ETS1_128458763_3596 | −0.02 | 7.07E−06 | 3′-UTR |
| RILPL1_promoter_2 | 0.02 | 8.19E−06 | Promoter | MIR8061_promoter | −0.02 | 7.19E−06 | Promoter |
| L1MA9_96879835_1052 | 0.06 | 8.52E−06 | LINE | GYPC_promoter | −0.02 | 7.55E−06 | Promoter |
| MAGI1_65353523_3551 | 0.03 | 8.77E−06 | 3′-UTR | LOC100506585_promoter | −0.02 | 7.68E−06 | Promoter |
| pcawg_14457 | 0.05 | 9.22E−06 | Enhancer | RAP1GAP2_promoter_2 | −0.02 | 8.14E−06 | Promoter |
| HOPX_56656298_208 | 0.07 | 1.08E−05 | 5′-UTR | PARVG_promoter_2 | −0.02 | 9.52E−06 | Promoter |
| MIR3_22825441_163 | 0.07 | 1.10E−05 | SINE | LINC02285_promoter | −0.02 | 9.73E−06 | Promoter |
| ADGRB2_promoter | 0.02 | 1.17E−05 | Promoter | LOC101929269_promoter | −0.02 | 1.03E−05 | Promoter |
| LOC105369306_promoter | 0.02 | 1.22E−05 | Promoter | ETS1_128458759_3600 | −0.02 | 1.06E−05 | 3′-UTR |
| L1P3b_26604165_2404 | 0.05 | 1.23E−05 | LINE | TMEM255B_113811902_5092 | −0.02 | 1.07E−05 | 3′-UTR |
| AluSq_11837774_279 | 0.05 | 1.23E−05 | SINE | pcawg_9331 | −0.02 | 1.08E−05 | Enhancer |
| pcawg_25683 | 0.04 | 1.26E−05 | Enhancer | GCNT1_promoter_3 | −0.02 | 1.10E−05 | Promoter |
| LINC02323_promoter | 0.02 | 1.28E−05 | Promoter | LILRB5_promoter_1 | −0.02 | 1.12E−05 | Promoter |
| SEMA6D_47717361_330 | 0.06 | 1.45E−05 | 5′-UTR | pcawg_17719 | −0.03 | 1.25E−05 | Enhancer |
| KISS1_promoter | 0.02 | 1.75E−05 | Promoter | LINC00945_promoter | −0.02 | 1.47E−05 | Promoter |
| AluSx1_114822924_283 | 0.07 | 1.97E−05 | SINE | MIR6891_promoter | −0.02 | 1.69E−05 | Promoter |
| ENPP2_119557407_112 | 0.07 | 2.07E−05 | 3′-UTR | L1PA11_81811171_2343 | −0.03 | 1.77E−05 | LINE |
| CHIA_promoter | 0.02 | 2.09E−05 | Promoter | LST1_promoter_4 | −0.02 | 1.79E−05 | Promoter |
| L2a_32901162_233 | 0.07 | 2.28E−05 | LINE | SIGLEC7_promoter | −0.02 | 1.94E−05 | Promoter |
| AluSx_26581408_310 | 0.07 | 2.31E−05 | SINE | ADGRE3_promoter | −0.03 | 1.99E−05 | Promoter |
| AluSg_102841705_294 | 0.06 | 2.41E−05 | SINE | MICB_promoter_3 | −0.02 | 2.09E−05 | Promoter |
| AluSx4_129776006_297 | 0.06 | 2.62E−05 | SINE | FFAR2_promoter | −0.02 | 2.32E−05 | Promoter |
| pcawg_11222 | 0.06 | 2.81E−05 | Enhancer | ITGB2-AS1_promoter | −0.02 | 2.48E−05 | Promoter |
| L2_105964244_407 | 0.06 | 3.18E−05 | LINE | MIR4752_promoter | −0.03 | 2.77E−05 | Promoter |
| AluY_33829005_283 | 0.05 | 3.53E−05 | SINE | pcawg_13352 | −0.03 | 2.96E−05 | Enhancer |
| AluY_26557181_296 | 0.06 | 3.86E−05 | SINE | MPZL3_118226688_3204 | −0.02 | 3.33E−05 | 3′-UTR |
| L1PA5_66020318_2717 | 0.03 | 3.89E−05 | LINE | MIR3155A_promoter | −0.02 | 3.36E−05 | Promoter |
| ZFYVE19_40812697_204 | 0.05 | 4.01E−05 | 3′-UTR | RPS6KA2-IT1_promoter | −0.02 | 3.46E−05 | Promoter |
| AluSx4_24276782_125 | 0.07 | 4.19E−05 | SINE | RNASE2_promoter | −0.02 | 3.55E−05 | Promoter |
| ZFP82_36391958_781 | 0.05 | 4.19E−05 | 3′-UTR | LOC100996583_promoter | −0.02 | 3.55E−05 | Promoter |
| L2a_79042856_82 | 0.06 | 4.27E−05 | LINE | MBP_promoter_1 | −0.02 | 3.61E−05 | Promoter |
| F2RL3_16888858_330 | 0.06 | 4.37E−05 | 5′-UTR | RILPL2_123410681_5208 | −0.01 | 3.68E−05 | 3′-UTR |
| pcawg_20413 | 0.02 | 4.70E−05 | Enhancer | LOC101448202_promoter | −0.02 | 3.98E−05 | Promoter |
| ARHGAP42_promoter | 0.02 | 4.74E−05 | Promoter | ADORA2A_promoter_2 | −0.02 | 4.01E−05 | Promoter |
| pcawg_21914 | 0.05 | 4.90E−05 | Enhancer | LINC00683_promoter | −0.02 | 4.19E−05 | Promoter |
| pcawg_29095 | 0.05 | 5.26E−05 | Enhancer | SNX20_promoter | −0.02 | 4.43E−05 | Promoter |
| VWA3B_98236572_157 | 0.06 | 5.43E−05 | 3′-UTR | TLR6_promoter | −0.02 | 4.62E−05 | Promoter |
| CpG-28717_1118176 | 0.05 | 5.54E−05 | CpG | LOC100129697_promoter | −0.02 | 4.67E−05 | Promoter |
| CTCF(Zf)_77038342_10 | 0.06 | 5.72E−05 | CTCF | GPIHBP1_promoter | −0.02 | 4.82E−05 | Promoter |
| ALDOA_30064495_288 | 0.07 | 6.08E−05 | 5′-UTR | TLR4_promoter | −0.02 | 5.19E−05 | Promoter |
| CpG-4864 | 0.04 | 6.46E−05 | CpG | LINC02241_promoter | −0.02 | 5.58E−05 | Promoter |
| L1PA17_101090721_226 | 0.06 | 6.92E−05 | LINE | GIMAP4_promoter | −0.02 | 5.89E−05 | Promoter |
| L2b_75269809_1308 | 0.05 | 7.31E−05 | LINE | CpG-29217 | −0.02 | 6.19E−05 | CpG |
| L3_53633720_366 | 0.06 | 7.52E−05 | LINE | RFLNB_439976_3186 | −0.02 | 6.30E−05 | 3′-UTR |
| PPARG_promoter_1 | 0.02 | 7.78E−05 | Promoter | IL17RA_17109819_5873 | −0.01 | 6.46E−05 | 3′-UTR |
| AluJo_102891593_319 | 0.07 | 7.95E−05 | SINE | NOVA2_45933732_6129 | −0.02 | 6.60E−05 | 3′-UTR |
| TPD52L1_promoter_2 | 0.02 | 8.01E−05 | Promoter | IL17RA_17109819_5874 | −0.01 | 6.68E−05 | 3′-UTR |
| pcawg_20113 | 0.05 | 8.35E−05 | Enhancer | CpG-18755 | −0.02 | 6.84E−05 | CpG |
| AluSz_102013953_291 | 0.05 | 8.50E−05 | SINE | pcawg_9239 | −0.01 | 6.94E−05 | Enhancer |
| CpG-23892 | 0.06 | 8.78E−05 | CpG | FCN1_134903230_6566 | −0.03 | 7.26E−05 | 3′-UTR |
| INSL4_5233876_1427 | 0.04 | 8.98E−05 | 3′-UTR | AGAP2-AS1_promoter | −0.02 | 7.61E−05 | Promoter |
| AluSp_102099550_297 | 0.06 | 9.20E−05 | SINE | FARS2_5827142_2049 | −0.02 | 7.77E−05 | 3′-UTR |
| PRKD2_46713861_139 | 0.06 | 9.42E−05 | 5′-UTR | IFITM4P_promoter | −0.02 | 7.91E−05 | Promoter |
| LINC01126_promoter | 0.02 | 9.66E−05 | Promoter | KCNJ15_promoter_3 | −0.02 | 8.11E−05 | Promoter |
| MIR3_123350970_156 | 0.05 | 9.83E−05 | SINE | KCNAB2_6098483_2703 | −0.02 | 8.25E−05 | 3′-UTR |

### Example 5: Detection 5hmC DNA Methylation State Change and RNA Transcriptional Measurement from the Same Biological Samples

The 5hmC DNA methylation state detection and whole RNA transcription profiling were performed from the same plasma samples using pregnant samples from cohort described in Example 1.

Differential RNA expression and differential DNA methylation analysis were performed using genes listed in Table 7 containing 20 highly predictive GA modeling and placenta genes discovered in RNA expression analysis.

TABLE 7
20 highly predictive GA modeling and placenta
genes discovered in RNA expression analysis.

CGA
CSH1
CAPN6
SVEP1
RBM3
PAPPA
STAT1
ANGPT2
LGALS14
EXPH5
CSHL1
BEX1
TACC2
VGLL3
SKIL
MCEMP1
UBE2L6
KISS1
HSD17B1
TACC1

Pairwise differential analysis for the samples using either RNA expression or DNA methylation change were performed, and results showed a high correlation between gene level RNA expression change and 5hmC DNA methylation change for the same gene locus.

Table 8 shows the p-values for separation between pairwise comparisons for samples collected at first, second, and third trimester for genes from Table 7. In many combinations, DNA methylation assays provided better separation between samples for each group analysis: first vs. second trimester, first vs. third trimester, and second vs. third trimester.

TABLE 8
The p-values for RNA levels and 5hmC DNA methylations separation
between samples collected at first, second, and third trimesters

| | 5hmC | | cfRNA | | |
|---|---|---|---|---|---|
| Gene | W-statistic | p-value | W-statistic | p-value | Comparison |
| SVEP1 | 104 | 4.26E−05 | 157 | 3.73E−03 | T2 vs T1 |
| PAPPA | 120 | 1.36E−03 | 177 | 1.27E−01 | T2 vs T1 |
| ANGPT2 | 240 | 2.36E−03 | 377 | 1.27E−01 | T2 vs T1 |
| KISS1 | 264 | 4.81E−03 | 326 | 6.13E−01 | T2 vs T1 |
| RBM3 | 272 | 5.85E−03 | 281 | 7.14E−01 | T2 vs T1 |
| CGA | 291 | 8.66E−03 | 431 | 8.21E−03 | T2 vs T1 |
| SVEP1 | 4 | 1.90E−13 | 20 | 4.29E−11 | T3 vs T1 |
| CGA | 54 | 3.74E−08 | 415 | 4.72E−02 | T3 vs T1 |
| KISS1 | 56 | 5.12E−08 | 224 | 8.78E−02 | T3 vs T1 |
| PAPPA | 71 | 2.36E−04 | 151 | 6.64E−03 | T3 vs T1 |
| ANGPT2 | 173 | 6.24E−03 | 451 | 6.64E−03 | T3 vs T1 |
| TACC1 | 223 | 8.42E−02 | 369 | 2.80E−01 | T3 vs T1 |
| SVEP1 | 10 | 4.40E−12 | 49 | 3.27E−08 | T3 vs T2 |
| VGLL3 | 30 | 9.04E−10 | 99 | 2.52E−05 | T3 vs T2 |
| CAPN6 | 36 | 3.10E−09 | 37 | 3.77E−09 | T3 vs T2 |
| TACC2 | 48 | 2.77E−08 | 77 | 1.94E−06 | T3 vs T2 |
| LGALS14 | 55 | 8.66E−08 | 116 | 1.38E−04 | T3 vs T2 |
| CGA | 59 | 1.60E−07 | 260 | 4.33E−01 | T3 vs T2 |
| PAPPA | 83 | 8.36E−03 | 119 | 1.60E−01 | T3 vs T2 |
| KISS1 | 89 | 8.33E−06 | 196 | 3.76E−02 | T3 vs T2 |
| TACC1 | 202 | 5.06E−02 | 363 | 2.13E−01 | T3 vs T2 |
| UBE2L6 | 208 | 6.69E−02 | 209 | 7.00E−02 | T3 vs T2 |
| ANGPT2 | 213 | 8.36E−02 | 371 | 1.60E−01 | T3 vs T2 |

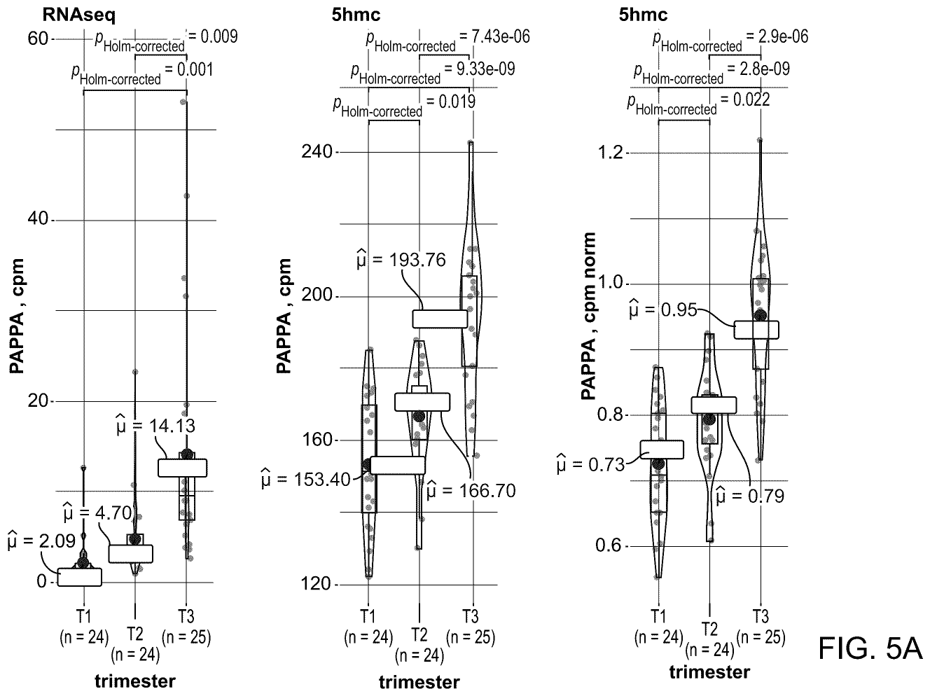

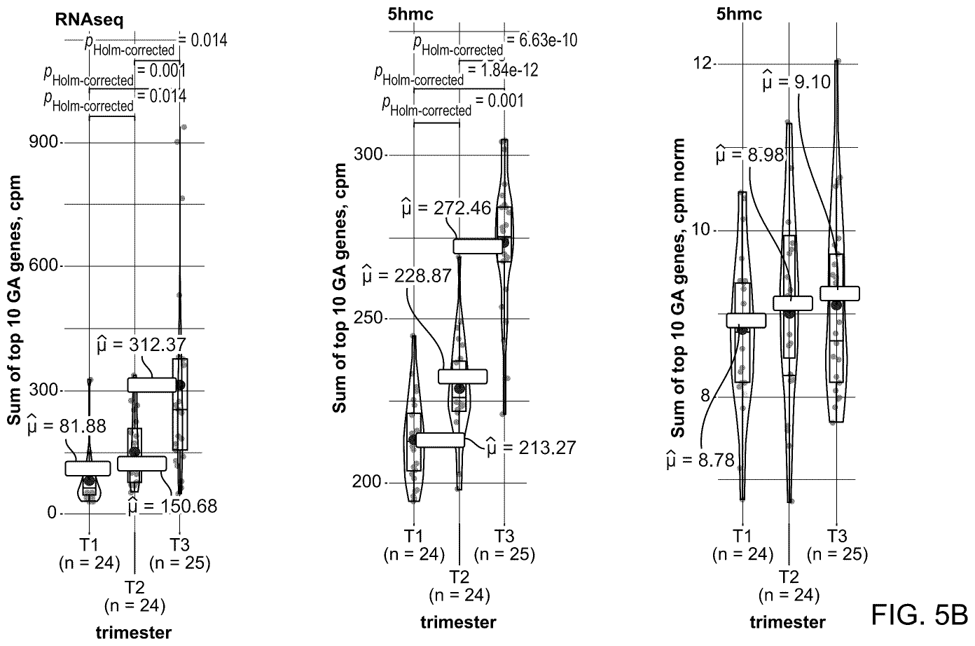

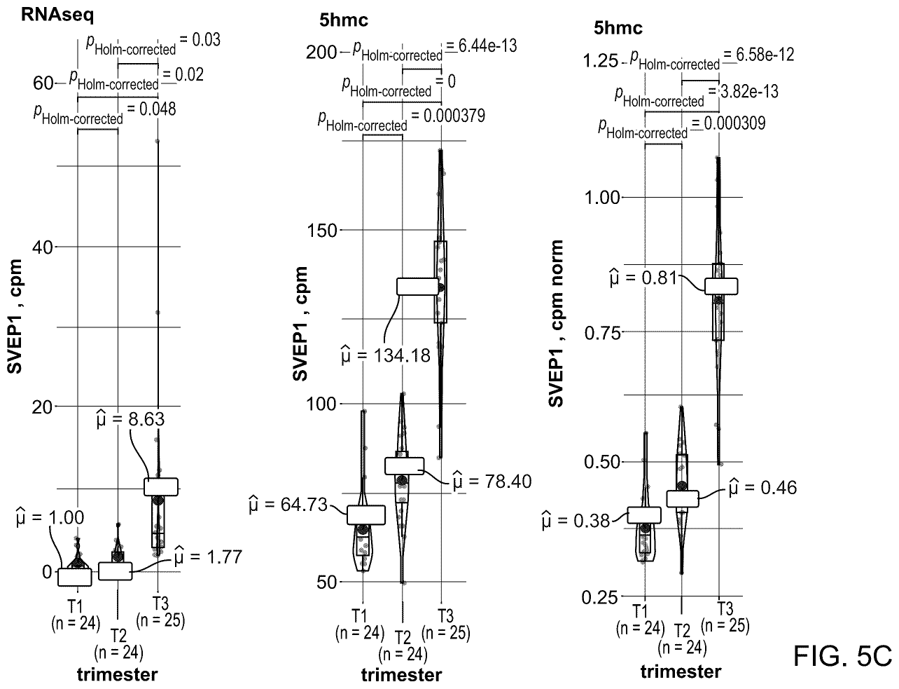

FIG. 5A shows signal separation based on detection of the PAPPA gene for a 5hmC DNA methylation state change compared to the PAPPA gene RNA expression change across the first, second, and third trimesters of pregnancy. FIG. 5B shows even higher separation for 5hmC DNA methylation change for sum of top 10 GA genes compared to RNA expression.

Samples collected at first trimester (9-12 weeks) and second trimester (18-21 weeks) were mostly poorly separated by RNA expression analysis. 5hmC DNA methylation analysis determined a set of 555 gene loci with more significant p-values for separation for the same samples. Table 9 shows a top 100 gene list allowing for a better separation of the first vs second trimester samples, by 5hmC DNA methylation than by RNA expression analysis.

TABLE 9
Top 100 gene list allowing for a better separation of the first vs
second trimester samples by 5hmC than by RNA expression analysis

| | 5hmC | | cfRNA | |
|---|---|---|---|---|
| Gene | W-statistic | Adj. p-value | W-statistic | Adj. p-value |
| SVEP1 | 96 | 3.41E−05 | 157 | 0.00373221 |
| RAB15 | 105 | 8.70E−05 | 276 | 0.64125051 |
| COL14A1 | 110 | 0.00014231 | 303 | 0.9604917 |
| PLAC8L1 | 114 | 0.00020806 | 311 | 0.83516677 |
| LGALS4 | 115 | 0.00022836 | 328 | 0.58548065 |
| RGL3 | 116 | 0.00025046 | 386 | 0.08734198 |
| DDX31 | 119 | 0.00032902 | 281 | 0.71382838 |
| TLL2 | 121 | 0.00039326 | 323 | 0.65552608 |
| VSX1 | 124 | 0.00051126 | 302 | 0.97628893 |
| RNF43 | 128 | 0.00071863 | 326 | 0.61309075 |
| DEFB136 | 130 | 0.00084867 | 295 | 0.92894894 |
| NPPC | 130 | 0.00084867 | 295 | 0.92894894 |
| FOSB | 131 | 0.00092137 | 304 | 0.94470969 |
| GLIS1 | 131 | 0.00092137 | 324 | 0.64125051 |
| CIDEC | 134 | 0.00117457 | 274 | 0.61309075 |
| LRP2 | 134 | 0.00117457 | 339 | 0.44473002 |
| RYR2 | 135 | 0.00127199 | 290 | 0.8506744 |
| PDGFC | 136 | 0.00137664 | 320 | 0.69908215 |
| DPY19L1 | 138 | 0.00160953 | 311 | 0.83516677 |
| FAM71D | 138 | 0.00160953 | 319 | 0.71382838 |
| MATN3 | 138 | 0.00160953 | 295 | 0.92894894 |
| STAU2 | 138 | 0.00160953 | 287 | 0.80434514 |
| PLIN1 | 140 | 0.00187728 | 398 | 0.05057517 |
| CRISP1 | 141 | 0.00202561 | 292 | 0.88185456 |
| PPIL1 | 141 | 0.00202561 | 270 | 0.55845159 |
| SLC34A2 | 141 | 0.00202561 | 376 | 0.13192576 |
| UBE2C | 142 | 0.00218437 | 175 | 0.01185011 |
| WDR63 | 142 | 0.00218437 | 308 | 0.88185456 |
| C14orf28 | 143 | 0.00235418 | 285 | 0.77381922 |
| CEP44 | 143 | 0.00235418 | 291 | 0.86623894 |
| DAOA | 143 | 0.00235418 | 309 | 0.86623894 |
| FAM131C | 143 | 0.00235418 | 295 | 0.92894894 |
| PSMB2 | 143 | 0.00235418 | 304 | 0.94470969 |
| TEX37 | 143 | 0.00235418 | 267 | 0.51906019 |
| RAB3B | 144 | 0.00253572 | 214 | 0.08734198 |
| ACSL6 | 145 | 0.00272969 | 259 | 0.42134749 |
| MYO3B | 145 | 0.00272969 | 240 | 0.23638246 |
| NSG1 | 145 | 0.00272969 | 298 | 0.97628893 |
| TTC23 | 145 | 0.00272969 | 280 | 0.69908215 |
| FGF5 | 146 | 0.00293679 | 295 | 0.92894894 |
| GABBR2 | 146 | 0.00293679 | 285 | 0.77381922 |
| MCMDC2 | 146 | 0.00293679 | 348 | 0.34528454 |
| FAM149B1 | 147 | 0.00315781 | 299 | 0.9920953 |
| OSBP2 | 147 | 0.00315781 | 207 | 0.06395103 |
| DNALI1 | 148 | 0.00339354 | 267 | 0.51906019 |
| GCG | 148 | 0.00339354 | 300 | 1 |
| IGFN1 | 148 | 0.00339354 | 251 | 0.33516738 |
| LDHB | 148 | 0.00339354 | 269 | 0.5451641 |
| YES1 | 148 | 0.00339354 | 254 | 0.366085 |
| GNG11 | 149 | 0.0036448 | 329 | 0.57189162 |
| LDB3 | 149 | 0.0036448 | 278 | 0.66992626 |
| SH3TC2 | 149 | 0.0036448 | 330 | 0.55845159 |
| AC034228.4 | 150 | 0.00391249 | 295 | 0.92894894 |
| HECW1 | 150 | 0.00391249 | 309 | 0.86623894 |
| SFRP4 | 150 | 0.00391249 | 362 | 0.22085944 |
| IQGAP2 | 151 | 0.00419749 | 367 | 0.18522612 |
| SLC16A12 | 151 | 0.00419749 | 270 | 0.55845159 |
| VWA8 | 151 | 0.00419749 | 308 | 0.88185456 |
| CLDN24 | 152 | 0.00450077 | 295 | 0.92894894 |
| KLHDC1 | 152 | 0.00450077 | 358 | 0.25264661 |
| LSM1 | 152 | 0.00450077 | 328 | 0.58548065 |
| MYOT | 152 | 0.00450077 | 288 | 0.81972179 |
| PLEKHA7 | 152 | 0.00450077 | 330 | 0.55845159 |
| ADAMTS19 | 153 | 0.00482331 | 301 | 0.9920953 |
| DEPDC1 | 153 | 0.00482331 | 176 | 0.0125775 |
| ERICH2 | 153 | 0.00482331 | 294 | 0.91321551 |
| FBXL2 | 153 | 0.00482331 | 343 | 0.39868903 |
| GRIP2 | 153 | 0.00482331 | 319 | 0.71382838 |
| OLA1 | 153 | 0.00482331 | 261 | 0.44473002 |
| OTOP3 | 153 | 0.00482331 | 291 | 0.86623894 |
| PLAG1 | 153 | 0.00482331 | 226 | 0.14262336 |
| ST7 | 153 | 0.00482331 | 319 | 0.71382838 |
| TMEM132E | 153 | 0.00482331 | 276 | 0.64125051 |
| ZBED2 | 153 | 0.00482331 | 347 | 0.35559063 |
| CAV2 | 154 | 0.00516614 | 253 | 0.35559063 |
| CD109 | 154 | 0.00516614 | 285 | 0.77381922 |
| CEP57L1 | 154 | 0.00516614 | 295 | 0.92894894 |
| FAM169A | 154 | 0.00516614 | 338 | 0.45668826 |
| NRP2 | 154 | 0.00516614 | 263 | 0.46882174 |
| DMRTB1 | 155 | 0.00553034 | 285 | 0.77381922 |
| ENY2 | 155 | 0.00553034 | 283 | 0.74363285 |
| PLEKHA6 | 155 | 0.00553034 | 206 | 0.06106851 |
| SAA2 | 155 | 0.00553034 | 256 | 0.38763525 |
| DYNC2LI1 | 156 | 0.00591703 | 297 | 0.9604917 |
| TRIP13 | 156 | 0.00591703 | 219 | 0.10785484 |
| ZNF507 | 156 | 0.00591703 | 266 | 0.50625009 |
| GLRA2 | 157 | 0.00632736 | 257 | 0.39868903 |
| PDK1 | 158 | 0.00676255 | 216 | 0.09514168 |
| BRIP1 | 159 | 0.00722385 | 273 | 0.59921498 |
| C9orf50 | 159 | 0.00722385 | 274 | 0.61309075 |
| SSUH2 | 159 | 0.00722385 | 373 | 0.14821115 |
| DNAJC6 | 160 | 0.00771256 | 274 | 0.61309075 |
| DUOXA1 | 160 | 0.00771256 | 341 | 0.42134749 |
| GLOD5 | 160 | 0.00771256 | 274 | 0.61309075 |
| IL20 | 160 | 0.00771256 | 297 | 0.9604917 |
| KBTBD8 | 160 | 0.00771256 | 298 | 0.97628893 |
| PIH1D3 | 160 | 0.00771256 | 295 | 0.92894894 |
| SEL1L3 | 160 | 0.00771256 | 277 | 0.65552608 |
| ZNF862 | 160 | 0.00771256 | 345 | 0.37676687 |

FIG. 5C shows representative RNA and 5hmC DNA methylation signals and p-values for SVREP1 gene for separation pregnancy samples between the first and second trimester.

### Example 6: Use of 5hmC DNA Methylation State to Estimate the Usability for Preeclampsia Prediction Model Based on RNA Expression

Data from Example 5 indicated a high correlation between gene RNA expression level and change in 5hmC DNA methylation status for corresponding gene locus. The data also indicated 5hmC DNA methylation measurement provides better separation for the same biological samples.

CLDN7, PAPPA2, SNORD14A, PLEKHH1, MAGEA10, TLE6, and FABP1 genes were determined to be predictive of preeclampsia using RNA expression modeling. The samples from first, second, and third trimesters were analyzed for RNA expression level and 5hmC DNA methylation status for corresponding gene locus for above gene list. P-values were calculated to estimate the sample groups separation based RNA expression level and 5hmC DNA methylation status.

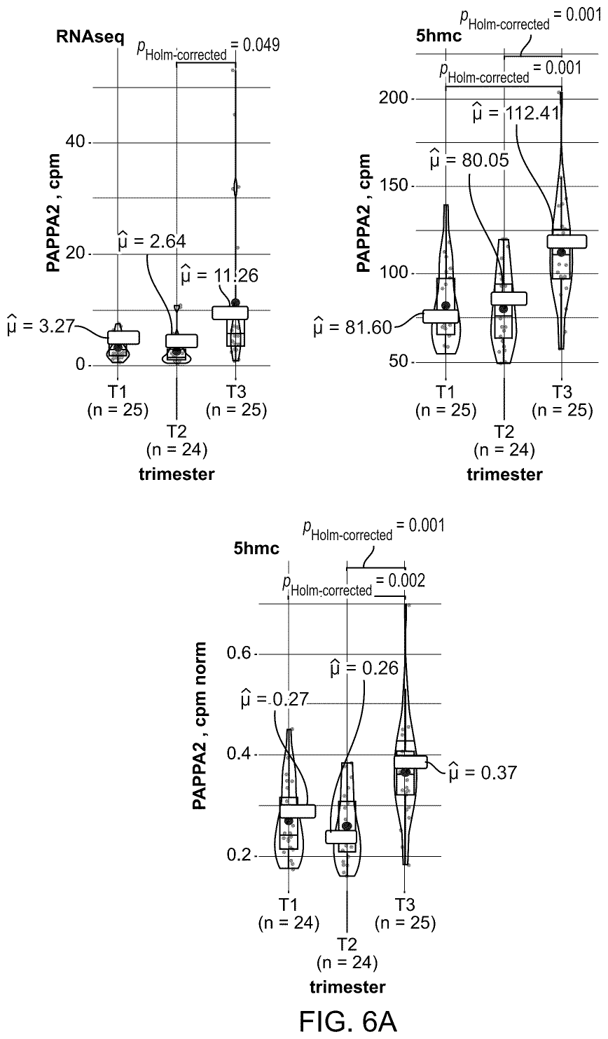

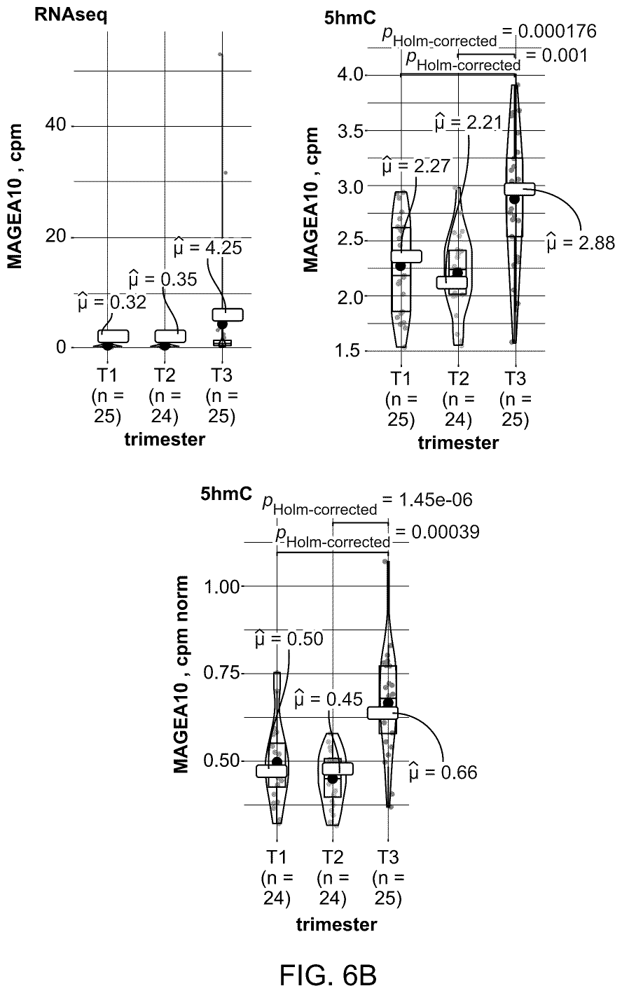

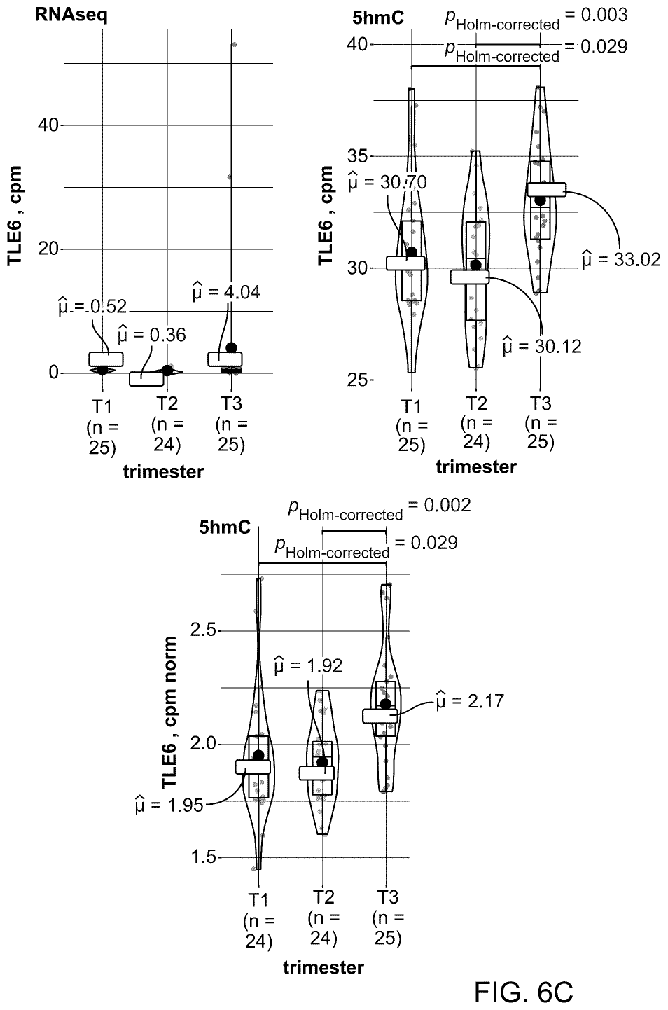

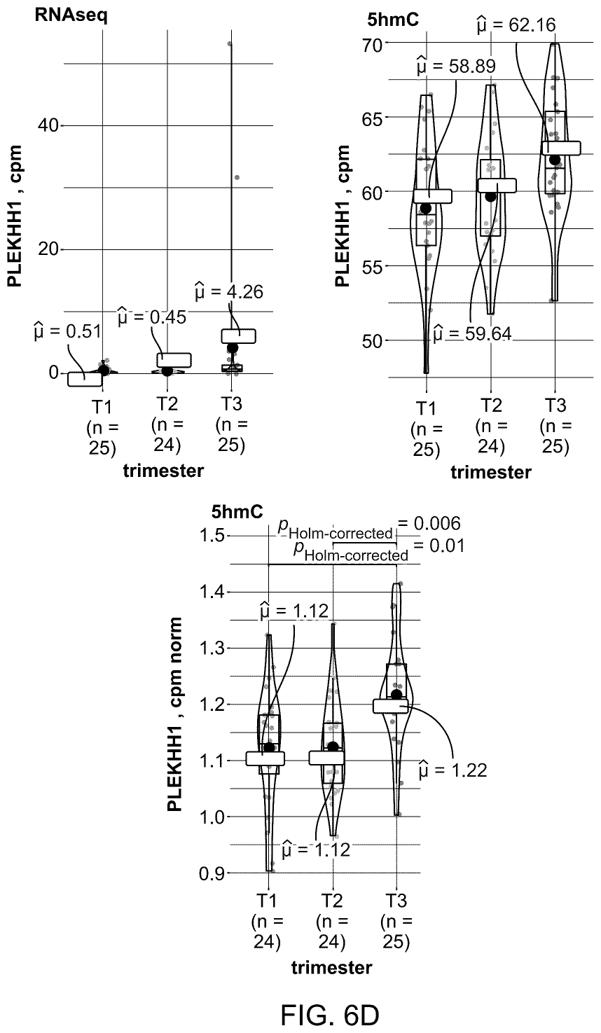

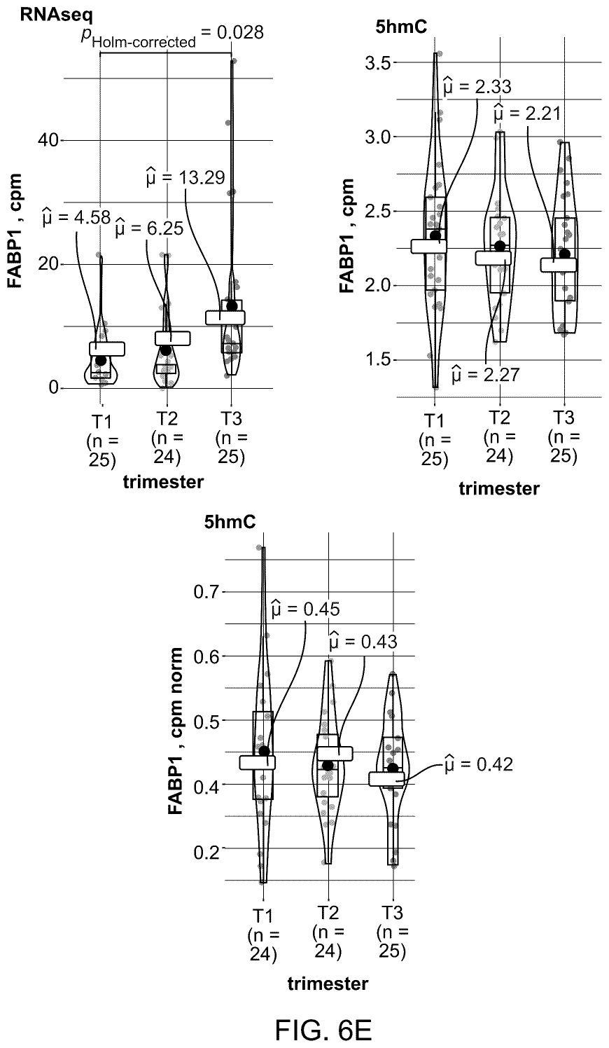

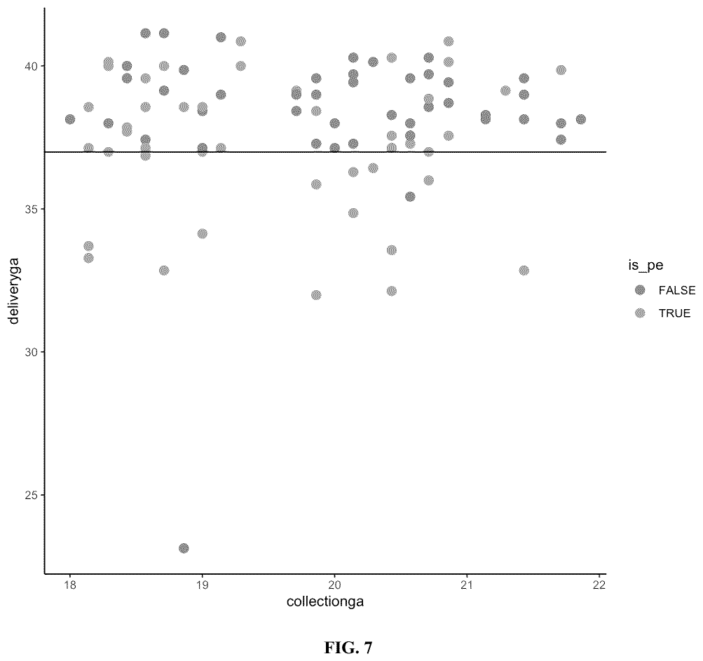

PAPPA2 (shown in FIG. 6A), MAGEA10 (shown in FIG. 6B), TLE6 (shown in FIG. 6C), PLEKHH1 9 (shown in FIG. 6D) genes show better separation by 5hmC DNA methylation between samples collected at different trimesters due to lower variance in measurement. Only FABP1 (sown in FIG. 6E) gene shows separation by RNA expression. These results indicate that 5hmC methylation assay may be used for preeclampsia modeling prediction using CLDN7, PAPPA2, SNORD14A, PLEKHH1, MAGEA10, TLE6, and FABP1 genes discovered by RNA expression.

FIG. 6A shows signal separation based on detection of the PAPPA2 gene for a 5hmC DNA methylation state change compared to PAPPA2 gene RNA expression change across the first, second, and third trimesters of pregnancy. FIG. 6B shows signal separation for detection of the MAGEA10 gene for a 5hmC DNA methylation state change compared to MAGEA10 gene RNA expression change across the first, second, and third trimesters of pregnancy. FIG. 6C shows signal separation for detection of the TLE6 gene for a 5hmC DNA methylation state change compared to TLE6 gene RNA expression change across the first, second, and third trimesters of pregnancy. FIG. 6D shows signal separation for detection of the PLEKHH1 gene for a 5hmC DNA methylation state change compared to PLEKHH1 gene RNA expression change across the first, second, and third trimesters of pregnancy. FIG. 6E shows signal separation for detection of the FABP1 gene for a 5hmC DNA methylation state change compared to FABP1 gene RNA expression change across the first, second, and third trimesters of pregnancy.

### Example 7

Methods for determining the genome-wide distribution of 5-hmC may be performed, for example, as described by Song et al., “Selective chemical labeling reveals the genome-wide distribution of 5-hydroxymethylcytosine”, Nature Biotechnology, 29, 68-72 (2011), which is incorporated by reference herein in its entirety.

Methods for detecting and analyzing nucleotide variants in genome or transcriptome may be performed, for example, as described by Song et al., “Mapping new nucleotide variants in the genome and transcriptome”, Nature Biotechnology, 30(11): 1107-1116, 2012, which is incorporated by reference herein in its entirety.

Methods for detecting and analyzing 5-hydroxymethylcytosine DNA may be performed, for example, as described by Robertson et al., “Pull-down of 5-hydroxymethylcytosine DNA using JBP1-coated magnetic beads”, Nature Protocols, 7(2): 340-50, 2012, which is incorporated by reference herein in its entirety.

Methods for detecting and analyzing 5-hydroxymethylcytosine signatures in cell-free DNA may be performed, for example, as described by Song et al., “5-Hydroxymethylcytosine signatures in cell-free DNA provide information about tumor types and stages”, Cell Research, 27, 1231-1242, 2017, which is incorporated by reference herein in its entirety.

Methods for detecting and analyzing placental 5-methylcytosine and 5-hydroxymethylcytosine profiles may be performed, for example, as described by Piyasena et al., “Placental 5-methylcytosine and 5-hydroxymethylcytosine patterns associate with size at birth”, Epigenetics, 10(8): 692-697, 2015, which is incorporated by reference herein in its entirety.

### Example 8: Use of 5hmC DNA Methylation State to Discover Signals Associated with Elevated Risk of Developing Preeclampsia

Using systems and methods of the present disclosure, a method of detecting and measuring the pregnancy-related DNA methylation signals in maternal plasma samples was developed to predict elevated preeclampsia risk from analysis of blood draws early in pregnancy, and preeclampsia development and diagnosis later in pregnancy.

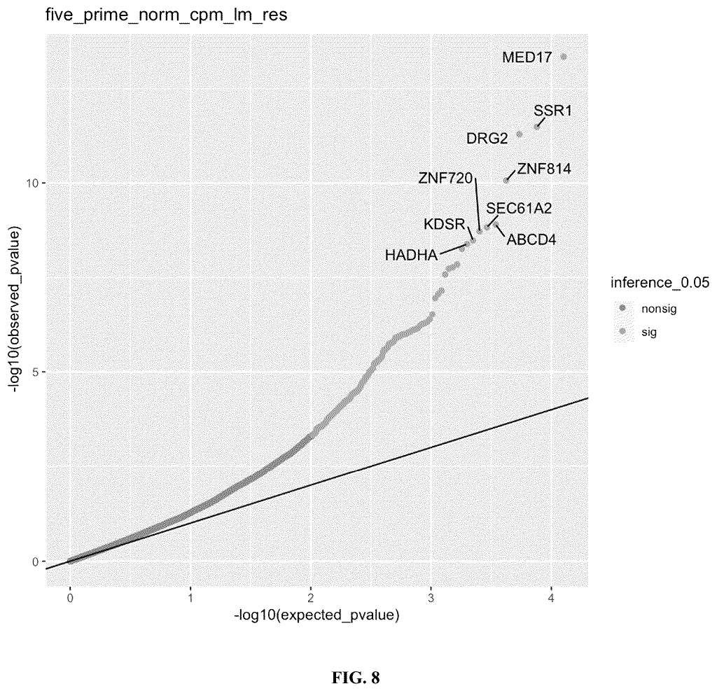

FIG. 7 shows a cohort of 89 subjects with 89 blood samples collected between 18-22 weeks of gestational age. The cohort contained 45 samples from subjects who later developed and had been diagnosed with preeclampsia and 44 healthy control subjects. Relevant demographics, comorbidities, and risk factors of the cohort are presented in Table 10.

TABLE 10
Demographic statistics for the preeclampsia cohort

| | Healthy | | PE | | Overall | |
|---|---|---|---|---|---|---|
| | (N = 44) | | (N = 45) | | (N = 89) | |
| major_race | | | | | | |
| asian | 1 | (2.3%) | 0 | (0%) | 1 | (1.1%) |
| black | 22 | (50.0%) | 25 | (55.6%) | 47 | (52.8%) |
| hispanic | 3 | (6.8%) | 4 | (8.9%) | 7 | (7.9%) |
| multiracial | 3 | (6.8%) | 2 | (4.4%) | 5 | (5.6%) |
| white | 15 | (34.1%) | 14 | (31.1%) | 29 | (32.6%) |
| is_ghtn | | | | | | |
| false | 31 | (70.5%) | 10 | (22.2%) | 41 | (46.1%) |
| true | 13 | (29.5%) | 35 | (77.8%) | 48 | (53.9%) |
| collectionga | | | | | | |
| Mean (SD) | 20.0 | (1.06) | 19.6 | (1.06) | 19.8 | (1.08) |
| Median [Min, Max] | 20.1 | [18.0, 21.9] | 19.3 | [18.1, 21.7] | 19.9 | [18.0, 21.9] |
| deliveryga | | | | | | |
| Mean (SD) | 38.4 | (2.64) | 37.3 | (2.44) | 37.8 | (2.59) |
| Median [Min, Max] | 38.6 | [23.1, 41.1] | 37.3 | [32.0, 40.9] | 38.3 | [23.1, 41.1] |
| m_age | | | | | | |
| Mean (SD) | 27.6 | (5.44) | 27.9 | (5.56) | 27.8 | (5.48) |
| Median [Min, Max] | 27.4 | [19.2, 38.6] | 26.8 | [18.7, 38.9] | 26.8 | [18.7, 38.9] |
| Missing | 1 | (2.3%) | 1 | (2.2%) | 2 | (2.2%) |
| m_bmi | | | | | | |
| Mean (SD) | 28.7 | (7.36) | 32.9 | (9.95) | 30.8 | (8.97) |
| Median [Min, Max] | 26.5 | [17.1, 44.9] | 31.6 | [19.1, 59.9] | 29.6 | [17.1, 59.9] |

DNA methylation states in each cfDNA sample extracted from 89 plasma samples were measured by performing whole-genome cell-free 5hmC sequencing based on a selective chemical labeling (hMe-Seal) technique, as described in (Cell Research volume 27, pages 1231-1242(2017)), which is incorporated by reference herein in its entirety. A total of 4,357,876 methylation features are distributed in 11 feature classes as described in Example 1.

For the first analysis, 5 feature classes (exons, introns, 5′- and 3′-untranslated regions (UTR), and promoters) were combined in a single genic group. The analysis of individual features and aggregate genic features was performed in a differential DNA methylation analysis. To identify elevated risk for preeclampsia, severe preeclampsia cases with delivery less than 37 week of gestational age (GA) were used, resulting in 42 healthy controls and 15 severe preeclampsia cases (FIG. 7). Table 11 shows the top 10 genic features that were significantly differently methylated in preeclampsia cases compared to controls. Most of them were associated with genes that are known to express RNA in reproductive organs.

TABLE 11
Top 10 genic features which differentiate between
preeclampsia vs control samples by 5hmC

| | | | Known |
|---|---|---|---|
| | | | expression in |
| | | | reproductive |
| Gene | P-value | Function | organs |
| ATG9B | 1.36E−05 | AUTOPHAGY-RELATED PROTEIN 9B | Yes |
| | | (PTHR13038:SF14) | |
| NCAM2 | 5.11E−05 | MICROFIBRILLAR-ASSOCIATED | Yes |
| | | PROTEIN 3-LIKE-RELATED | |
| | | (PTHR12231:SF231) | |
| MRGPRX1 | 7.37E−05 | MAS-RELATED G-PROTEIN COUPLED | Yes |
| | | RECEPTOR MEMBER X1 | |
| | | (PTHR11334:SF22) | |
| USHBP1 | 1.43E−04 | USHER SYNDROME TYPE-1C PROTEIN- | Yes |
| | | BINDING PROTEIN 1 (PTHR23347:SF5) | |
| LMBRD1 | 2.69E−04 | LYSOSOMAL COBALAMIN TRANSPORT | Yes |
| | | ESCORT PROTEIN LMBD1 | |
| | | (PTHR16130:SF2) | |
| FAM47A | 2.72E−04 | PROTEIN FAM47A (PTHR47415:SF4) | Yes |
| FAM90A26 | 3.17E−04 | PROTEIN FAM90A10P-RELATED | Yes |
| | | (PTHR16035:SF14) | |
| OTUD6A | 3.17E−04 | OTU DOMAIN-CONTAINING PROTEIN 6A | |
| | | (PTHR12419:SF13) | |
| GFRAL | 3.35E−04 | GDNF FAMILY RECEPTOR ALPHA-LIKE | Yes |
| | | (PTHR10269:SF1) | |
| KRTAP5-6 | 3.36E−04 | KERATIN-ASSOCIATED PROTEIN 5-6 | Yes |
| | | (PTHR23262:SF231) | |

In the second analysis, all 45 preeclampsia cases and 44 healthy controls were used. 5′ UTR features were used to construct a linear model, where a signal from features belonging to one gene were modeled as random effects for all samples. Table 12 shows 184 genes with differentially methylated 5′ UTRs at a false discovery rate <5%, which differentiate between preeclampsia cases and healthy controls. FIG. 8 shows a quantile-quantile (QQ) plot for differential DNA methylation status that illustrates differentially methylated 5′-UTR in preeclampsia cases.

TABLE 12
Top 184 genes with differentially methylated 5′-UTR
features which differentiate between preeclampsia vs
control samples at an FDR < 0.05

| Gene feature | P-value |
|---|---|
| ABCD4 | 1.26E−09 |
| ACAA1 | 4.92E−06 |
| ADAM17 | 1.41E−04 |
| ADCK5 | 7.84E−07 |
| ADGRE1 | 2.91E−04 |
| ADGRG1 | 6.54E−05 |
| AK2 | 9.62E−06 |
| AL049834.1 | 2.55E−04 |
| AL391650.1 | 1.16E−06 |
| ALG10B | 4.53E−04 |
| APLNR | 1.71E−06 |
| APOBEC3B | 4.40E−05 |
| APOBEC3F | 1.32E−04 |
| AREG | 2.78E−04 |
| ARPC5 | 1.22E−04 |
| BCAS4 | 1.24E−04 |
| BCHE | 5.29E−05 |
| BLOC1S5 | 6.07E−05 |
| BSDC1 | 9.21E−05 |
| C10orf143 | 1.10E−04 |
| C11orf49 | 8.88E−05 |
| C1orf54 | 5.75E−05 |
| CASTOR1 | 6.10E−07 |
| CCDC12 | 1.91E−04 |
| CCDC13 | 3.14E−04 |
| CCDC163 | 5.24E−07 |
| CCDC36 | 8.09E−06 |
| CCHCR1 | 4.31E−04 |
| CCNYL1 | 1.51E−04 |
| CCR3 | 1.83E−04 |
| CD96 | 1.28E−05 |
| CDH13 | 3.52E−05 |
| CEP78 | 1.86E−08 |
| CHP1 | 2.81E−04 |
| CIAPIN1 | 2.33E−05 |
| CLEC5A | 6.35E−05 |
| CLK4 | 3.10E−04 |
| CLTA | 3.32E−05 |
| CMPK1 | 2.65E−05 |
| CNIH1 | 1.08E−04 |
| COL13A1 | 5.57E−09 |
| CPN2 | 1.53E−04 |
| CRISP2 | 6.07E−05 |
| CUTA | 2.22E−04 |
| CWF19L1 | 4.37E−06 |
| CYP4A22 | 3.48E−05 |
| DDC | 2.36E−04 |
| DDX50 | 3.85E−05 |
| DHRS12 | 4.18E−04 |
| DHX35 | 6.09E−05 |
| DNM1 | 3.72E−04 |
| DPAGT1 | 1.78E−05 |
| DPY19L4 | 1.01E−04 |
| DRG2 | 5.09E−12 |
| DYNC2H1 | 8.56E−05 |
| EEF1AKMT2 | 3.18E−04 |
| EFEMP1 | 2.59E−06 |
| EMC1 | 2.73E−04 |
| ERMN | 9.91E−06 |
| FAM133B | 1.63E−05 |
| FAM185A | 8.49E−06 |
| FAM57A | 2.60E−06 |
| FAM86C1 | 3.75E−05 |
| FAM98A | 2.41E−06 |
| FAU | 8.24E−07 |
| FCRL3 | 4.48E−04 |
| FUT11 | 3.13E−04 |
| GATB | 8.65E−05 |
| GIMAP4 | 5.29E−05 |
| GOLGA2 | 2.78E−04 |
| GSDMB | 3.92E−04 |
| GTF2IRD2 | 7.75E−05 |
| HADHA | 4.15E−09 |
| HEATR1 | 4.75E−04 |
| HMOX2 | 3.25E−04 |
| HOXA9 | 1.20E−04 |
| IPO4 | 1.53E−06 |
| ITGA11 | 3.19E−04 |
| KCNH1 | 4.15E−07 |
| KCTD13 | 4.26E−04 |
| KDSR | 3.33E−09 |
| LGALS3BP | 1.11E−05 |
| LGALS9C | 1.12E−07 |
| LGI1 | 4.88E−07 |
| LRPPRC | 3.90E−05 |
| LSM14A | 2.91E−04 |
| LSM4 | 4.59E−04 |
| LTO1 | 2.27E−06 |
| LY9 | 2.25E−04 |
| LZTR1 | 1.01E−06 |
| MAGT1 | 4.71E−05 |
| MAPKAPK5 | 1.19E−04 |
| MAS1 | 1.84E−04 |
| MBD4 | 5.01E−05 |
| MED17 | 4.54E−14 |
| METTL26 | 1.43E−08 |
| MFSD8 | 8.66E−08 |
| MPI | 2.47E−05 |
| MRPL4 | 7.04E−08 |
| MRPS35 | 3.23E−05 |
| MT1E | 4.38E−04 |
| MUC1 | 7.19E−05 |
| MZB1 | 1.71E−04 |
| NELL1 | 1.39E−04 |
| NFAT5 | 6.20E−06 |
| NMNAT1 | 8.58E−05 |
| NOP16 | 1.07E−04 |
| NSFL1C | 3.01E−06 |
| NSG2 | 2.42E−04 |
| NT5C3B | 1.72E−08 |
| NUP160 | 4.75E−04 |
| NUP98 | 5.49E−07 |
| OGG1 | 1.38E−05 |
| OPRM1 | 1.43E−04 |
| OR6B1 | 2.88E−04 |
| ORC5 | 4.35E−04 |
| P3H1 | 4.59E−06 |
| PABPC4 | 1.02E−06 |
| PARP1 | 1.59E−04 |
| PIGT | 5.96E−06 |
| PLD2 | 1.84E−05 |
| PML | 5.44E−05 |
| PNPO | 1.17E−06 |
| PODXL | 9.58E−05 |
| POGLUT1 | 7.12E−07 |
| PPP2R3C | 2.04E−04 |
| PRSS36 | 2.43E−04 |
| PSG1 | 1.26E−05 |
| RAB34 | 1.67E−06 |
| RAD51AP1 | 9.21E−07 |
| RAD51C | 1.38E−05 |
| RASL10A | 1.70E−04 |
| RBBP4 | 2.28E−04 |
| RBM19 | 2.73E−04 |
| RFC1 | 9.12E−06 |
| RHCG | 3.33E−05 |
| RINT1 | 2.97E−04 |
| RNF121 | 1.07E−06 |
| RNF212 | 2.99E−04 |
| RPL14 | 3.01E−05 |
| RPL27 | 1.61E−04 |
| RPS2 | 3.02E−07 |
| RPS3A | 1.06E−04 |
| SEC61A2 | 1.49E−09 |
| SF3B1 | 7.74E−05 |
| SFTPC | 3.90E−06 |
| SLC22A23 | 7.26E−05 |
| SLC2A1 | 2.81E−04 |
| SLC2A11 | 2.52E−04 |
| SLC2A14 | 1.66E−04 |
| SLC35G1 | 3.23E−04 |
| SLC9B1 | 4.06E−04 |
| SLTM | 1.54E−04 |
| SON | 4.99E−06 |
| SPIN3 | 8.92E−07 |
| SSR1 | 3.25E−12 |
| STMP1 | 7.49E−05 |
| TARSL2 | 1.38E−04 |
| TCL1A | 1.27E−06 |
| TESC | 3.55E−04 |
| THAP1 | 4.55E−04 |
| TM7SF2 | 1.89E−05 |
| TMEM234 | 1.12E−04 |
| TMPRSS4 | 2.38E−04 |
| TOR2A | 7.82E−05 |
| TPCN2 | 3.48E−05 |
| TUBG1 | 1.26E−06 |
| TYW1 | 1.00E−06 |
| UBXN1 | 5.80E−06 |
| USP29 | 5.96E−05 |
| VXN | 2.01E−05 |
| WDR24 | 7.53E−07 |
| WSB1 | 1.31E−04 |
| ZHX3 | 4.72E−04 |
| ZNF268 | 1.83E−06 |
| ZNF286A | 3.96E−05 |
| ZNF483 | 2.90E−05 |
| ZNF669 | 2.12E−04 |
| ZNF720 | 1.92E−09 |
| ZNF738 | 5.37E−05 |
| ZNF778 | 2.63E−04 |
| ZNF785 | 1.85E−06 |
| ZNF814 | 8.69E−11 |
| ZSWIM7 | 2.63E−08 |

Additional analysis shows that 11 genes presented in Table 13 encode proteins involved in remodeling of extracellular matrix, which is a key functional component of the process of placentation. It is noted that preeclampsia is often considered to be a placental disease, and that it is feasible that an early implantation deficiency is associated with elevated risk of developing preeclampsia.

TABLE 13
Genes with differentially methylated 5′-UTRs that
are associated with extracellular matrix remodeling

| Gene | P-value | Protein | Class |
|---|---|---|---|
| LGALS9C | 1.12E−07 | GALECTIN-9C (PTHR11346:SF80) | extracellular matrix |
| | | | protein(PC00102) |
| P3H1 | 4.59E−06 | PROLYL 3-HYDROXYLASE 1 | extracellular matrix |
| | | (PTHR14049:SF5) | glycoprotein(PC00100) |
| EFEMP1 | 2.59E−06 | EGF-CONTAINING FIBULIN- | extracellular matrix structural |
| | | LIKE EXTRACELLULAR MATRIX | protein(PC00103) |
| | | PROTEIN 1 (PTHR24039:SF33) | |
| COL13A1 | 5.57E−09 | COLLAGEN ALPHA-1(XIII) | extracellular matrix structural |
| | | CHAIN (PTHR24023:SF871) | protein(PC00103) |
| GOLGA2 | 2.78E−04 | GOLGIN SUBFAMILY A | membrane traffic |
| | | MEMBER 2 (PTHR10881:SF58) | protein(PC00150) |
| CNIH1 | 1.08E−04 | PROTEIN CORNICHON | membrane traffic |
| | | HOMOLOG 1 (PTHR12290:SF10) | protein(PC00150) |
| SSR1 | 3.25E−12 | TRANSLOCON-ASSOCIATED | membrane traffic |
| | | PROTEIN SUBUNIT ALPHA | protein(PC00150) |
| | | (PTHR12924:SF0) | |
| CLTA | 3.32E−05 | CLATHRIN LIGHT CHAIN A | vesicle coat protein(PC00235) |
| | | (PTHR10639:SF1) | |
| CLTC | 2.90E−05 | CLATHRIN HEAVY CHAIN 1 | vesicle coat protein(PC00235) |
| | | (PTHR10292:SF7) | |
| ATP6AP2 | 5.37E−05 | RENIN RECEPTOR | transmembrane signal |
| | | (PTHR13351:SF5) | receptor(PC00197) |
| CPN2 | 1.53E−04 | CARBOXYPEPTIDASE N | transmembrane signal |
| | | SUBUNIT 2 (PTHR24373:SF271) | receptor(PC00197) |
| OR6B1 | 2.88E−04 | OLFACTORY RECEPTOR 6B1 | transmembrane signal |
| | | (PTHR26453:SF284) | receptor(PC00197) |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-122. (canceled)

123. A method for determining that a pregnant subject has or is at elevated risk of having a pregnancy-related complication, comprising:
(a) assaying a cell-free biological sample obtained or derived from said subject to determine at least one deoxyribonucleic acid (DNA) methylation level of at least one pregnancy-associated genomic region, wherein said at least one pregnancy-associated genomic region is differentially methylated in a first population of pregnant subjects with a pregnancy involving said pregnancy complication as compared to a second population of pregnant subjects without a pregnancy involving said pregnancy complication;
(b) computer processing said at least one DNA methylation level of said at least one pregnancy-associated genomic region determined in (a) (i) against at least one reference methylation level of said at least one pregnancy-associated genomic region or (ii) with a trained machine learning algorithm; and
(c) determining, based at least in part on said computer processing in (b), that said pregnant subject has said elevated risk of having said pregnancy complication.

124. The method of claim 123, wherein said assaying in (a) comprises nucleic acid sequencing or a 5-hydroxymethylcytosine (5hmC) DNA enrichment assay.

125. The method of claim 123, wherein (a) further comprises assaying 5-methylcytosine (5mC) and/or 5hmC in the cell-free biological sample.

126. The method of claim 123, further comprising assaying RNA transcripts in said cell-free biological sample derived from said subject, and computer processing said RNA transcripts to determine that said pregnant subject has said elevated risk of having said pregnancy complication.

127. The method of claim 123, wherein said pregnancy-related complication is selected from the group consisting of pre-term birth, a pregnancy-related hypertensive disorder, gestational diabetes, a congenital disorder of a fetus of said subject, ectopic pregnancy, spontaneous abortion, stillbirth, a post-partum complication, hyperemesis gravidarum, hemorrhage or excessive bleeding during delivery, premature rupture of membrane, premature rupture of membrane in pre-term birth, placenta previa, intrauterine/fetal growth restriction, macrosomia, a neonatal condition, and a fetal development complication.

128. The method of claim 127, wherein said pregnancy-related complication is a molecular sub-type of pre-term birth.

129. The method of claim 128, wherein said molecular subtype of pre-term birth is selected from the group consisting of history of prior pre-term birth, spontaneous pre-term birth, ethnicity specific pre-term birth risk, and pre-term premature rupture of membrane (PPROM).

130. The method of claim 123, further comprising administering a treatment to said pregnant subject based at least in part on said determining in (c).

131. The method of claim 130, wherein said treatment comprises cervical cerclage or a drug selected from the group consisting of a corticosteroid, a progestational agent, insulin, an antibiotic, a tocolytic drug, a calcium channel blocker, a cyclo-oxygenase inhibitor, an oxytocin antagonist, a betamimetic drug, magnesium sulfate, magnesium chloride, and magnesium oxide.

132. The method of claim 123, wherein said at least one pregnancy-associated genomic region is associated with gestational age, wherein said at least one pregnancy-associated genomic region is selected from the group consisting of genes listed in Table 3, non-genic loci listed in Table 4, genes listed in Table 5, non-genic loci listed in Table 6, genes listed in Table 7, and genes listed in Table 9.

133. The method of claim 127, wherein said pregnancy-related complication is a molecular sub-type of preeclampsia.

134. The method of claim 133, wherein said molecular subtype of preeclampsia is selected from the group consisting of history of chronic or pre-existing hypertension, presence or history of gestational hypertension, presence or history of mild preeclampsia, presence or history of severe preeclampsia, presence or history of eclampsia, and presence or history of HELLP syndrome.

135. The method of claim 127, wherein said at least one pregnancy-associated genomic region is associated with preeclampsia, wherein said at least one pregnancy-associated genomic region is selected from the group consisting of genomic and non-genomic or aggregated loci listed in Table 11, Table 12, and Table 13, and CLDN7, PAPPA2, SNORD14A, PLEKHH1, MAGEA10, TLE6, and FABP1 genes.

136. The method of claim 123, wherein said cell-free biological sample is selected from the group consisting of cell-free ribonucleic acid (cfRNA), cell-free deoxyribonucleic acid (cfDNA), cell-free fetal DNA (cffDNA), plasma, serum, urine, saliva, and amniotic fluid, and a derivative thereof.

137. The method of claim 136, wherein said cell-free biological sample is said plasma.

138. The method of claim 123, further comprising fractionating a whole blood sample of said pregnant subject to obtain said cell-free biological sample.

139. The method of claim 123, wherein said assaying in (a) further comprises quantitative polymerase chain reaction (qPCR).

140. The method of claim 123, wherein said pregnant subject is asymptomatic for said pregnancy complication.

141. The method of claim 123, wherein (b) further comprises computer processing clinical health data of said pregnant subject to determine that said pregnant subject has said elevated risk of having said pregnancy complication.

142. The method of claim 123, wherein (a) further comprises (i) subjecting said cell-free biological sample to conditions that are sufficient to isolate, enrich, or extract a set of DNA molecules, and (ii) assaying said set of DNA molecules.

143. The method of claim 142, further comprising using primers or probes to selectively enrich said set of DNA molecules corresponding to a panel of genomic regions.

144. The method of claim 142, wherein (a) further comprises subjecting said set of DNA molecules to nucleic acid sequencing to generate a set of sequencing reads.

145. The method of claim 142, wherein (a) further comprises subjecting said set of DNA molecules to nucleic acid amplification.

146. The method of claim 123, wherein said trained machine learning algorithm comprises a deep learning algorithm, a support vector machine (SVM), a neural network, or a Random Forest.

147. A method for determining that a pregnant subject has or is at elevated risk of having a pregnancy-related complication, comprising:
(a) using a first assay to process a cell-free biological sample obtained or derived from said pregnant subject to generate a first dataset comprising RNA transcriptional biomarkers;
(b) using a second assay to process a cell-free biological sample obtained or derived from said pregnant subject to generate a second dataset comprising DNA methylation biomarkers;
(c) computer processing at least said first dataset and said second dataset to determine that a pregnant subject has or is at elevated risk of having said pregnancy-related complication.
